(19) United States
(12) Patent Application Publication
(10) Pub. No.: US 2026/0284030 A1
(43) Pub. Date: Sep. 24, 2026

(54) METHODS OF TREATING HEART FAILURE

(71) Applicant: THE CHILDREN'S MEDICAL CENTER CORPORATION, Boston, MA (US)

(72) Inventors: Denisa D. WAGNER, Dover, MA (US); Lukas A. HEGER, Cambridge, MA (US); Nicolas SCHOMMER, Brookline, MA (US)

(73) Assignee: THE CHILDREN'S MEDICAL CENTER CORPORATION, Boston, MA (US)

(21) Appl. No.: 19/132,665

(22) PCT Filed: Nov. 21, 2023
(86) PCT No.: PCT/US2023/080681

§ 371 Date: May 23, 2025

Related U.S. Application Data
(60) Provisional application No. 63/427,592, filed on Nov. 23, 2022.

(51) Int. Cl. A61K 31/4545; A61P 9/04

(52) U.S. Cl. CPC A61K 31/4545; A61P 9/04

(57) ABSTRACT

The technology described herein is directed to administering a PAD4 inhibitor to treat a subject diagnosed with having, or at risk of having heart failure, e.g., HFpEF, diastolic heart failure, or diastolic dysfunction. Compositions comprising a PAD4 inhibitor is also described herein.

### CROSS REFERENCE TO RELATED APPLICATIONS

This application claims benefit under 35 U.S.C. § 119(e) of U.S. Provisional Application No. 63/427,592, filed Nov. 23, 2022, the contents of which are incorporated herein by reference in its entirety.

### GOVERNMENT SUPPORT

This invention was made with government support under Grant No. R35HL135765 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 7, 2023, is named 701039-000104WOPT_SL.txt and is 62,337 bytes in size.

### TECHNICAL FIELD

The technology described herein relates to methods of treating heart failure.

### BACKGROUND

Heart failure is a syndrome, a group of signs and symptoms, caused by impairment of the heart's function to pump blood. There are different types of heart failures which are characterized by the failure of different parts of the heart to pump blood. Examples include right-sided heart failure that affects the right heart, left-sided heart failure that affects the left heart, and biventricular failure that affects both sides of the heart. Left-sided heart failure can occur with reduced ejection fraction or with a preserved ejection fraction. Heart failure is not the same as cardiac arrest in which blood flow stops altogether due to failure of the heart to pump effectively.

Heart failure with preserved ejection fraction (HFpEF) is a form of heart failure in which the ejection fraction—the percentage of the volume of blood ejected from the left ventricle with each heartbeat divided by the volume of blood when the left ventricle is maximally filled—is normal, defined as greater than 50%. Diastolic heart failure is a stiff left heart ventricle. When your left heart ventricle is stiff, it doesn't relax properly between heartbeats. Diastolic heart failure can lead to decreased blood flow and other complications. Diastolic dysfunction is a problem with diastole, the first part of your heartbeat. Typically, your lower heart chambers relax and fill with blood during diastole. Diastolic dysfunction occurs when your lower heart chambers don't relax as they should. Over time, the dysfunction can lead to diastolic heart failure.

### SUMMARY

The technology described herein is directed a method of treating or preventing heart failure comprising administering a PAD4 inhibitor to a subject in need thereof.

In one embodiment of any aspect, the subject in need thereof has been diagnosed with having heart failure. In one embodiment of any aspect, the subject in need thereof has been diagnosed with being at risk of having heart failure.

In one embodiment of any aspect, the PAD4 inhibitor is an antibody reagent, an inhibitory nucleic acid, or a small molecule.

In one embodiment of any aspect, the PAD4 inhibitor inhibits PAD4 expression and/or activity. In one embodiment of any aspect, PAD4 expression and/or activity is inhibited by at least 10%, 20%, 30%, 40%, 50%, 60%, 70%, 80%, 90%, 99% or more as compared to expression and/or activity prior to administration.

In one embodiment of any aspect, the small molecule is JBI-589:

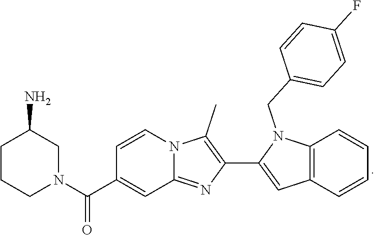 [SMILES: Cc1c(-c2cc3ccccc3n2Cc2ccc(F)cc2)nc2cc(C(=O)N3CCC[C@@H](N)C3)ccn12]

In one embodiment of any aspect, the administering is selected from the group comprising topically, intravascularly, intravenously, intraarterially, intratumorally, intramuscularly, subcutaneously, intraperitoneally, intranasally, or orally.

In one embodiment of any aspect, the heart failure comprises diastolic dysfunction, diastolic heart failure, and heart failure with preserved ejection fraction (HFpEF). In one embodiment of any aspect, the heart failure is HFpEF. In one embodiment of any aspect, the HFpEF arises as a result of autoimmune diseases, Diabetes Mellitus, hypertension, aging, and obesity.

In one embodiment of any aspect, the method further comprises the step, prior to administering, diagnosing the subject of having or at risk of having heart failure.

In one embodiment of any aspect, the method further comprises the step, prior to administering, receiving the results of an assay that diagnoses the subject of having or at risk of having heart failure.

In one embodiment of any aspect, the subject is a mammal. In one embodiment of any aspect, the mammal is human.

Another aspect of the technology described herein is a method of treating or preventing heart failure, the method comprising administering a PAD4 inhibitor to a subject in need thereof, wherein the PAD4 inhibitor is JBI-589.

Another aspect of the technology described herein is a composition for the treatment or prevention of heart failure comprising a PAD4 inhibitor and a pharmaceutically acceptable carrier.

Another aspect of the technology described herein is a composition for treating or preventing heart failure, the composition comprising a PAD4 inhibitor, wherein the PAD4 inhibitor is JBI-589.

Another aspect of the technology described herein is a use composition comprising a PAD4 inhibitor for the treatment of prevention of heart failure, wherein the PAD4 inhibitor is JBI-589.

### BRIEF DESCRIPTION OF THE DRAWINGS

FIGS. 1A-1F: Mice with Collagen Induced Arthritis (CIA) develop diastolic dysfunction and myocardial hypertrophy.

FIG. 1A shows a timecourse illustrating the ratio of early (E) to late (A) diastolic filling velocities across the mitral valve as measured by pulsed wave doppler (E/A ratio; left Y axis; -). Clinical severity of rheumatoid arthritis (RA Score; right y axis; ---) in DBA/1J mice with CIA and a healthy control respectively. (n=6) Arrows indicate time point of inoculation/booster with type II collagen and (*) complete/incomplete Freud's adjuvant.’

FIG. 1B examines left ventricular ejection fraction in DBA/1J mice with CIA and a healthy control respectively, demonstrated over the course of time. (n=6)

FIG. 1C shows the E/A ratio and ejection fraction (EF) as assessed by echocardiography in mice with CIA and healthy control at day 56. (n=12)

FIG. 1D depicts representative flow patterns, acquired using pulsed-wave Doppler echocardiography, depicting the velocities over the mitral valve at indicated timepoints in mice with CIA and healthy control respectively.

FIG. 1E examines deceleration of time reflecting the amount of time needed to equalize the pressure difference between the left atrium (LA) and the left ventricle (LV) or the time interval from the peak of the E-wave to its projected baseline and isovolumetric relaxation time (IVRT) a marker for myocardial relaxation measuring the time for crossover between the LA and LV pressures as assessed by echocardiography in arthritic CIA mice and healthy controls at day 56. (n=12)

FIG. 1F shows heart-weight normalized to tibia-length in CIA mice and healthy control group after 56 days. (n=9)

For FIG. 1A-1F, Data are mean±SEM *P<0.05, **P<0.01, ***P<0.001; Unpaired Student's T test (IVRT: Mann-Whitney U-test).

FIGS. 2A-2F: Left ventricular remodeling and fibrosis in mice with CIA drive development of clinically relevant diastolic dysfunction similar to Heart Failure with Preserved Ejection Fraction (HFpEF).

FIG. 2A depicts representative images of LV sections stained with Wheat germ agglutinin (WGA) for cell membrane and DAPI staining of DNA to measure cardiomyocyte cell size defined by cross section area illustrating cardiac hypertrophy. (n=6)

FIG. 2B examines Left ventricular mass (LVM) a parameter estimating total LV weight which was measured using echocardiography. LVM was calculated as difference between the epicardium delimited volume and the LV chamber volume multiplied by an estimate of myocardial density. Diastolic LV posterior wall thickness (LVPWd) was measured at end diastole as a measure of LV geometry and expansion. (n=12)

FIG. 2C shows representative LV section images of Masson's trichrome staining for fibrotic tissue in a mouse with CIA and a healthy control respectively. Comparative analysis of total area of fibrotic tissue in LV sections. (n=9/8)

FIG. 2D depicts representative images of Masson's trichrome staining with perivascular fibrosis and quantitative analysis. (For each mouse 5 peripheral vessels chosen at random were quantified and their average was used for comparative analysis; n=10)

FIG. 2E determines the quantification of Collagen I and vasculature (CD31⁺ cells) in LV. Representative images are shown. (n=5)

FIG. 2F examines plasma levels of Brain natriuretic peptide, a hormone produced by the body when the heart is enlarged, as measured by ELISA on day 56 in mice with CIA and a healthy control group. (n=12)

For FIGS. 2A-2F, Data are mean±SEM. *P<0.05, **P<0.01, ***P<0.001; Mann-Whitney U-test (F: Student's t test).

FIGS. 3A-3E: Neutrophils are activated both in the circulation and in myocardium of mice with CIA.

FIG. 3A examines representative immunofluorescence images of isolated neutrophils from CIA− or healthy control mice. Neutrophils from CIA mice are preactivated (H3Cit⁺) and show a propensity for spontaneous NET formation. Representative microscopic pictures of isolated neutrophils are shown. Healthy control (left) and CIA (right). *=activated H3Cit⁺ neutrophil. (n=6)

FIG. 3B shows a graph, illustrating as a function of time the neutrophil-to-lymphocyte ratio (NLR) a marker of hyperinflammatory response, measured in peripheral blood in DBA/1J mice with CIA and a healthy control respectively. (n=6)

FIG. 3C examines gating and quantification of infiltrating CD45⁺ and Ly6G⁺ cells using Flow Cytometry (FACS) in healthy control and CIA myocardial tissue. (n=14)

FIG. 3D determines quantitative comparison of tissue levels of Interleukin 1beta, an inflammatory cytokine mediating fibrosis, measured using ELISA. (n=9)

FIG. 3E examines representative LV sections of CIA mice and healthy control mice stained for DAPI⁺, Ly6G⁺, and H3Cit⁺ cells as well as a representative image of an activated neutrophil in the right panel with co-staining of Ly6G⁺ and H3Cit⁺. Comparative analysis of total number of Ly6G⁺ and H3cit⁺ cells in LV heart sections of CIA mice and healthy controls. (n=8)

Data are mean±SEM *P<0.05, **P<0.01, ***P<0.001; Student's t test.

FIGS. 4A-4E: Thromboinflammation, endothelial activation and activated neutrophils outside the vascular bed are present in RA mice hearts.

FIG. 4A examines representative LV sections and quantification of von Willebrand Factor (VWF) staining, an endothelial adhesive protein deposited in the vasculature. (n=7)

FIG. 4B shows endothelial activation leads to extrusion of endothelial-anchored VWF within the vascular lumen. Arrows indicate VWF near the vessel wall and * indicates intraluminal VWF aggregates; L=Lumen of vessel.

FIG. 4C depicts immunofluorescence staining in LV sections of mice with CIA and healthy control mice for accumulating CD42b⁺ cells indicative of (micro) thrombosis in the vasculature a major feature of thromboinflammation.

FIG. 4D examines macroscopic picture of an aggregate of CD42b⁺ platelets in myocardium of a mouse with CIA and quantification of total number of aggregates per LV section. (n=5)

FIG. 4E depicts immunofluorescence staining in LV sections of mice with CIA for H3Cit⁺ cells in relation to CD31⁺ signal with quantification of H3Cit⁺ cells. (For each mouse 5 peripheral and 5 perivascular cross section areas were chosen blindly and quantified and the resulting total average used for comparative analysis; n=5)

For FIGS. 4A-4E, data are mean±SEM. *P<0.05, **P<0.01, ***P<0.001; Mann-Whitney U-test.

FIGS. 5A-5G: Treatment with an orally available PAD4 inhibitor preserves LV diastolic function and dampens disease progression in mice with RA.

FIG. 5A shows a timecourse illustrating the ratio of early (E) to late (A) diastolic filling velocities across the mitral valve as measured by pulsed wave Doppler echocardiography (E/A ratio; left Y axis; -) in mice with CIA and mice with CIA treated with PAD4 inhibitor. (n=8) Clinical severity of rheumatoid arthritis (RA Score; right y axis; ---) in DBA/1J mice with CIA and mice with CIA treated with PAD4 inhibitor. (n=8)

FIG. 5B examines representative flow patterns, acquired using pulsed-wave Doppler echocardiography, depicting the velocities over the mitral valve at an indicated timepoint in a mouse with CIA and a mouse with CIA treated with PAD4 inhibitor.

FIG. 5C shows E/A ratio and ejection fraction (EF) as assessed by echocardiography in mice with CIA and mice with CIA treated with PAD4 inhibitor respectively at day 56. (n=8)

FIG. 5D examines representative LV section images of Sirius Red fast Green Staining Kit and comparative analysis of total fibrotic tissue in CIA and CIA treated with PAD4 inhibitor respectively. Arrows indicate fibrotic tissue. (n=8)

FIG. 5E shows deceleration of time reflecting the duration for equalizing the pressure difference between the left atrium (LA) and the left ventricle (LV) and isovolumetric relaxation time (IVRT) a marker for myocardial relaxation measuring the time for crossover between the LA and LV pressures as assessed by echocardiography in arthritic CIA mice and mice with CIA treated with PAD4 inhibitor. (n=7)

FIG. 5F examines representative immunofluorescence staining of LV sections from a mouse with CIA and a healthy control for CD31⁺ cells and Collagen 1. Quantification of total Collagen I deposition per LV section. (n=5/6)

FIG. 5G determines LV mass as determined by echocardiography and heart-weight normalized to tibia-length of mice with CIA and mice with CIA treated with PAD4 inhibitor after 56 days. (n=8)

For FIGS. 5A-5G, Data are mean±SEM *P<0.05, **P<0.01, ***P<0.001; Paired Student's t test (D, E, F: Wilcoxon matched pairs signed rank test).

FIGS. 6A-6D: Treatment of mice with CIA using a PAD4 inhibitor decreases neutrophil infiltration as well as endothelial activation in mice with CIA after 26 days of treatment.

FIG. 6A shows representative immunostained LV sections and quantification of double positive (Ly6G⁺ and H3cit⁺) cells indicative of activated neutrophils in myocardium of mice with CIA and mice with CIA treated with PAD4 inhibitor. (n=8)

FIG. 6B determines quantification of infiltrating CD45⁺ and Ly6G⁺ cells in myocardial tissue of mice with CIA and mice with CIA treated with PAD4 inhibitor respectively, assessed by flow cytometry (FACS). (n=7)

FIG. 6C examines representative LV sections and quantification of DAPI⁺ and VWF⁺ signal in myocardium of mice with CIA and mice with CIA treated with PAD4 inhibitor.

FIG. 6D examines myocardial tissue levels of Interleukin 1beta, an inflammatory cytokine mediating fibrosis, and secreted by neutrophils in mice with CIA and mice with CIA treated with PAD4 inhibitor.

For FIGS. 6A-6D, Data are mean±SEM *P<0.05, **P<0.01, ***P<0.001; Paired Student's t test (B: Wilcoxon matched pairs signed rank test).

FIG. 7A examines left ventricular mass, a parameter estimating total LV weight was calculating using Vevo LAB ultrasound analysis software as the difference between the epicardium-delimited volume and the LV chamber volume multiplied by an estimate of myocardial density. Diastolic LV posterior wall thickness (LVPWd) was measured at the end diastole as a measure of LV geometry and expansion (n=12).

FIG. 7B shows plasma levels of brain natriuretic peptide, as measured by ELISA on day 56 in mice with CIA and a healthy control group. (n=12) as determined by ELSA and heart weight normalized to tibia length of mice with CIA and mice with CIA treated with the PAD4 inhibitor after 56 d. (n=8). Data are mean±SEM. *p<0.05, **p<0.01, ***p<0.001; paired t test.

FIGS. 8A-8E depict signs of chronic inflammation and neutrophil activation are present in mice with CIA. Data are mean±SEM. *P<0.05, **P<0.01, ***P<0.001; Unpaired Student's T test (C, D: Mann-Whitney U-test).

FIG. 8A shows body weight (n=9) and peripheral blood neutrophil count at the day of sacrifice in healthy control and in mice with CIA. (n=11).

FIG. 8B shows plasma levels of Interleukin 6 (116) in peripheral blood of mice with CIA and healthy control mice as assessed by ELISA. (n=8)

FIG. 8C shows representative LV section images of Masson's trichrome staining for fibrotic tissue in a mouse with CIA and a healthy control respectively.

FIG. 8D shows plasma levels of double strand (ds) DNA in CIA and healthy control mice at week 3 and 5 respectively. (n=5)

FIG. 8E shows Western blots of plasma levels of H3cit at week 3 and 5 in healthy control and CIA mice respectively. (n=5)

FIGS. 9A-9E exhibit production of the profibrotic mediator transforming growth factor-β (TGF-β) is elevated in heart tissue of mice with CIA. Data are mean±SEM. *P<0.05, **P<0.01, ***P<0.001; Mann-Whitney U-test.

FIG. 9A shows representative immunostained LV sections labeled for DAPI⁺, CD68⁺ a marker of monocyte cell lineage and TGF-β⁺ proprotein a protein with a pivotal role in the pathogenesis of fibrosis of a mouse with CIA and healthy control muse. (n=6)

FIG. 9B shows quantification and comparative analysis of CD68⁺ positive and TGF-β⁺ cells in myocardium of CIA mice and healthy control mice respectively. (n=6)

FIG. 9C shows heart tissue levels of TGF-β as assessed by ELISA in tissue lysates. (n=5)

FIG. 9D shows representative immunostained LV sections labeled for DAPI⁺, alpha smooth muscle actin a marker of myofibroblasts and CD31V cells in a mouse with CIA and healthy control mouse. Arrows indicate Myofibroblasts positive for alpha smooth muscle actin and negative for CD31 while “V” illustrates vessels.

FIG. 9E shows total number of alpha smooth muscle actin positive and CD31 negative cells indicative of activated myofibroblasts in CIA mice and healthy control mice.

FIGS. 10A-10D depict how treatment with JBI-589 improves health parameters, decreases neutrophil activation and alters pro-fibrotic signaling. Data are mean±SEM. *P<0.05, **P<0.01, ***P<0.001; Mann-Whitney U-test.

FIG. 10A shows body weight of CIA mice and mice treated with PAD4 inhibitor at day 56. (n=8)

FIG. 10B shows Interleukin 6 (Il-6) plasma levels in healthy control, mice with CIA and mice with CIA treated with PAD4 inhibitor respectively. (n_{control}=5; n_{CIA/CIA+PAD41}=8)

FIG. 10C shows representative immunostained LV sections labeled for DAPI⁺, alpha smooth muscle actin and CD31 positive cells in mice with CIA mice and mice with CIA treated with PAD4 inhibitor. Arrows indicate cells positive for alpha smooth muscle actin and in being distant from vessel (V) indicative of myofibroblasts. (n=5)

FIG. 10D and FIG. 10E Tissue levels of TGF-β as determined immunostaining of LV sections. (n=5)

### DETAILED DESCRIPTION

The various aspects described herein are based in part on the inventors' discovery that PAD4 inhibitors can treat heart failure with preserved ejection fraction (HFpEF), diastolic heart failure, and diastolic disfunction. Inhibiting PAD4 reverses the effect of the thickening of the left ventricle. Accordingly, in one aspect, provided herein is a method for treating diastolic heart failure, HFpEF, and diastolic dysfunction in a subject diagnosed with heart failure by inhibiting PAD4 directly or by inhibiting the expression of a nucleic acid encoding PAD4 in a cardiomyocyte. One aspect provided herein provides a method of treating or preventing heart failure comprising administering a PAD4 inhibitor to a subject in need thereof. Another aspect provided herein provides a method of treating or preventing heart failure, the method comprising administering a PAD4 inhibitor to a subject in need thereof, wherein the PAD4 inhibitor is JBI-589.

In one embodiment, the subject in need thereof has been diagnosed with having heart failure. In one embodiment, the subject in need thereof has been diagnosed with being at risk of having heart failure.

In one embodiment, the method further comprises the step, prior to administering, diagnosing the subject of having or at risk of having heart failure. In one embodiment, the method further comprises the step, prior to administering, receiving the results of an assay that diagnoses the subject of having or at risk of having heart failure.

Protein-arginine deiminase (PADs) contain PAD1-4 and PAD6. They are an essential post-translational modification enzyme that converts protein arginine residues to noncoding citrulline residues in a calcium-dependent manner. Citrullination alters the structure, function, and binding proteins of target proteins, thereby enabling their participation in a variety of physiological and pathological processes. PADs need to be activated to cause citrullination. PAD1 and PAD3 are mainly distributed in the epidermis and hair follicles and are involved in epidermal differentiation and homeostasis. Moreover, PAD1 distribution has been detected in the uterus. PAD2 and PAD4 are widely expressed in human tissues. PAD2 is distributed in the brain, skeletal muscle, spleen and immune cells and is involved mainly in regulating nervous system function and immune cell differentiation, while PAD4 is expressed in hematopoietic stem cells and immune cells (e.g., neutrophils, monocytes, macrophages and natural killer (NK) cells). Notably, PAD4 is the only PAD isoenzyme that carries a standardized nuclear localization sequence (NLS), which enables it to target a range of nuclear proteins, such as histones (H1, H2A, H2B, H3 and 114), NPM1, ING4, P300/CBP, and lamin C.

Protein-arginine deiminase type-4 (PAD4), is a human protein which in humans is encoded by the PADI4 gene. This gene is a member of a gene family which encodes enzymes responsible for the conversion of arginine to citrulline residues. It can also play a role in granulocyte and macrophage development leading to inflammation and immune response. PAD4 is normally found in the cytoplasm, nucleus, and in cytoplasmic granules of eosinophils and neutrophils. PAD5 is the mouse homolog to PAD4 in humans.

PAD4 referred to in this aspect, and all aspects and embodiments described herein in this application, comprises the nucleotide sequences of PADI4 with NCBI nucleotide sequence IDs:
NG_023261.3 (SEQ ID NO: 1, PAD4 genomic sequence), NM_012387.3 (SEQ ID NO: 2, PAD4 mRNA sequence), NP_036519.2 (SEQ ID NO: 3, PAD4 protein sequence).

In some preferred embodiments of any one of the aspects described herein, PAD 4 is encoded on human chromosome 1. PAD4, also referred to as PAD or PADI4 in the art, encodes enzymes responsible for the conversion of arginine residues to citrulline residues. The encoded protein is known to respond to diverse cellular stresses to regulate apoptosis, inflammation, and cell cycle mediation. Mutations in this gene are associated with a variety of human diseases such as pseudohypoaldosteronism and adrenal cortical carcinoma.

Embodiments of the various aspects described herein include administering an inhibitor of PAD4 to a subject. The inhibitor can directly bind with PAD4 or with a nucleic acid encoding PAD4. Exemplary inhibitors that can be used include, but are not limited to, nucleic acids, antibodies, and small molecules.

### Nucleic Acid Inhibitors of PAD4

In some embodiments of any of the aspects, the agent that inhibits PAD4 is an inhibitory nucleic acid. In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like. In some embodiments of any of the aspects, the inhibitory nucleic acid can be a silencing RNA (siRNA), microRNA (miRNA), or short hairpin RNA (shRNA). Inhibitory nucleic acids can also include guide sequence molecules (e.g., a guide RNA) that function, e.g., in combination with an enzyme, to induce insertions, deletions, indels, and/or mutations of a target, thereby inhibiting the expression of the target.

Generally, a nucleic acid inhibitor comprises a nucleotide sequence that is substantially complementary to at least a portion of a nucleic acid encoding PAD4. For example, the nucleic acid inhibitor comprises a nucleotide sequence that is substantially complementary to at least a portion of a nucleotide sequence selected from SEQ ID NOs: 1-3. In some embodiments, the nucleic acid inhibitor comprises a nucleotide sequence that is substantially complementary to at least a portion of a nucleotide sequence selected from SEQ ID NOs: 1-2.

In some embodiments of any of the aspects, the nucleic acid inhibitor comprises a sequence at least 15 nucleotides in length that is substantially complementary to at least a portion of a nucleotide sequence selected from SEQ ID NOs: 1-3. For example, the nucleic acid inhibitor comprises a sequence 15, 16, 17, 18, 19, 20, 21, 22, 23, 24, or 25 nucleotides in length that is substantially complementary to at least a portion of a nucleotide sequence selected from SEQ ID NOs: 1-2.

In some embodiments, the nucleic acid inhibitor inhibits PAD4. For example, the nucleic acid inhibitor comprises a sequence 15, 16, 17, 18, 19, 20, 21, 22, 23, 24, or 25 nucleotides in length that is substantially complementary to at least a portion of a nucleotide sequence selected from SEQ ID NOs: 1-2.

In some embodiments of any of the aspects, an iNA comprises a sequence that is complementary to at least a portion of a target sequence described herein. In some embodiments of any of the aspects, an iNA comprises a sequence at least 15 nucleotides in length that is complementary to at least a portion of a target sequence described herein. In some embodiments of any of the aspects, an iNA comprises a sequence at least 20 nucleotides in length that is complementary to at least a portion of a target sequence described herein.

In some embodiments of any of the aspects, an iNA comprises a sequence that is the reverse complement to at least a portion of a target sequence described herein. In some embodiments of any of the aspects, an iNA comprises a sequence at least 15 nucleotides in length that is the reverse complement to at least a portion of a target sequence described herein. In some embodiments of any of the aspects, an iNA comprises a sequence at least 20 nucleotides in length that is the reverse complement to at least a portion of a target sequence described herein.

In some embodiments of any of the aspects, an iNA comprises a sequence that can specifically hybridize to at least a portion of a target sequence described herein. In some embodiments of any of the aspects, an iNA comprises a sequence at least 15 nucleotides in length that can specifically hybridize to at least a portion of a target sequence described herein. In some embodiments of any of the aspects, an iNA comprises a sequence at least 20 nucleotides in length that can specifically hybridize to at least a portion of a target sequence described herein.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target, e.g., PAD4. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g., siRNA, shRNA, miRNA, and/or amiRNA, which are well known in the art. One skilled in the art would be able to design further siRNA, shRNA, or miRNA to target the nucleic acid sequence of PAD4 (e.g., SEQ ID NO: 2), e.g., using publicly available design tools. siRNA, shRNA, or miRNA is commonly made using companies such as Dharmacon (Layfayette, CO) or Sigma Aldrich (St. Louis, MO).

In some embodiments of the various aspects described herein, the inhibitory nucleic acid is a guide nucleic acid (gNA). As used herein, the terms “guide nucleic acid,” “guide sequence,” “crRNA,” “guide RNA,” “single guide RNA,” “gRNA” or “CRISPR guide sequence” refer to a nucleic acid comprising a sequence that determines the specificity of an enzyme, e.g., the Cas DNA binding protein of a CRISPR/Cas system, to a polynucleotide target. The gNA can comprise a polynucleotide sequence with at least partial complementarity with a target nucleic acid sequence, sufficient to hybridize with the target nucleic acid sequence and to direct sequence-specific binding of an enzyme, e.g, a nuclease, to the target nucleic acid sequence.

In some embodiments, the enzyme directed by the gNA is a gene-editing protein, e.g., any nuclease that induces a nick or double-strand break into a desired recognition site. Such enzymes can be native or engineered. These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA: nuclease complex binds a target polynucleotide sequence having a protospacer adjacent motif (“PAM”) and a protospacer, which is a sequence complementary to a portion of the gRNA. The recognition and binding of the target polynucleotide by the gRNA: nuclease complex induces cleavage of the target.

Any CRISPR-associated nuclease can be used in the system and methods of the invention. CRISPR nuclease systems are known to those of skill in the art, e.g. Cas9, Cas12, Casl2a, or the like, see Patents/applications 8,993,233, US 2015/0291965, US 2016/0175462, US 2015/0020223, US 2014/0179770, 8,697,359; 8,771,945; 8, 795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; 8,865,406; 8,871,445; each of which are incorporated by reference in their entirety. The nuclease can also be a phage Cas nuclease, e.g., Cas(D (e.g., Pausch et al. Science 369:333-7 (2020); which is incorporated by reference herein in its entirety).

The full-length guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, a nucleic acid strand is less than about 75, 50, 45, 40, 35, 30, 25, 20, 15, 12, or fewer nucleotides in length. For example, the guide nucleic acid sequence is 10-30 nucleotides long.

In addition to a sequence that is complementary to a target nucleic acid, in some embodiments, the gNA also comprises a scaffold sequence. Expression of a gNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gNA may be referred to as a single guide RNA (sgRNA).

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off_Spotter™; and Synthego™; which are available on the world wide web).

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2--NH—CH2--, —CH2--N(CH3)--O—CH2--[known as a methylene (methylimino) or MMI backbone], —CH2--O—N(CH3)-CH2--, —CH2--N(CH3)--N(CH3)-CH2-- and —N(CH3)--CH2-CH2--[wherein the native phosphodiester backbone is represented as —O—P—O—CH2--].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2)·nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2--N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

### Antibody Inhibitors of PAD4

Antibodies that specifically bind PAD4 can be used for inhibition in vivo, in vitro, or ex vivo. The PAD4 inhibitory activity of a given antibody, or, for that matter, any PAD4 inhibitor, can be assessed using methods known in the art or described herein. Specific binding is typically defined as binding that does not recognize other antigens, such as a protein, nucleotide, chemical residue, etc., at a detectable level in an assay used. Accordingly, in some embodiments of any one of the aspects described herein, the inhibitor of the PAD4 is an antibody or an antigen binding fragment thereof. Generally, the antibody or the antigen binding fragment thereof binds an epitope on the PAD4 such that the binding inhibits a function and/or activity of the PAD4.

Antibodies that can be used according to the methods described herein, include complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred.

Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Thus, antibodies useful in the methods described herein include, but are not limited to, naturally occurring antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen.

Antibodies can also be raised against a nucleotide, polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., 1988. While both polyclonal and monoclonal antibodies can be used in the methods described herein, it is preferred that a monoclonal antibody is used where conditions require increased specificity for a particular protein.

The term “intrabodies” as used herein, refers to a method wherein to target intracellular endogenous proteins as described in U.S. Pat. No. 6,004,940. Briefly, the method comprises the intracellular expression of an antibody capable of binding to the target. A DNA sequence is delivered to a cell, the DNA sequence contains a sufficient number of nucleotides coding for the portion of an antibody capable of binding to the target operably linked to a promoter that will permit expression of the antibody in the cell(s) of interest. The antibody is then expressed intracellularly and binds to the target, thereby disrupting the target from its normal actions.

“Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

Antibody inhibitors of PAD4 can include polyclonal and monoclonal antibodies and antigen-binding derivatives or fragments thereof.

In some embodiments of any one of the aspects described herein, the inhibitor is an anti-PADI4/PAD4 antibody or an antigen binding fragment thereof. Generally, the antibody or the antigen binding fragment thereof binds an epitope on the PAD4 such that the binding inhibits a function and/or activity of the PAD4. In some embodiments of any one of the aspects described herein, the antibody binds to polypeptide of comprising an amino acid sequence selected from the group consisting of SEQ ID NO: 3. Exemplary anti-PADI/PAD4 antibodies are commercially available and include, but are not limited to anti-PADI4/PAD4 rabbit monoclonal EPR20706 (Cat. No. #ab214810, Abcam, Waltham, MA); anti-PADI4/PAD4 mouse monoclonal OTI4H5 (Cat No. #ab128086, Abcam, Waltham, MA); PADI4 (A-11), (Cat. No. #sc-365369, Santa Cruz Biotechnology, Dallas, TX); anti-PADI4 monoclonal antibody, clone 5E9 (Cat. No. #CABT-21614MH, Creative Diagnostics, New York, NY); anti-PADI4 monoclonal antibody, clone 516 (Cat. No. #DCABH-1961, Creative Diagnostics, New York, NY); anti-PADI4 monoclonal antibody, clone 4F2 (Cat. No. #DCABH-3701, Creative Diagnostics, New York, NY).

Methods of obtaining antibodies against an antigen are well known in the art and available to one of skill in the art.

### Small Molecule Inhibitors of PAD4

In some embodiments of any one of the aspects described herein, the inhibitor of the PAD4 is a small molecule. Any inhibitors of PAD4 can be used in the methods described herein. For example, in some embodiments, a PAD4 inhibitor can be a small molecule inhibitor. Small molecule inhibitors of PAD4 are known in the art (see, for example, Luo et al. Biochemistry 2006; U.S. Pat. No. 7,964,636; and U.S. Patent Publications 2007/0276040 and 2011/0142868; each of which is incorporated by reference herein in its entirety) and include, by way of non-limiting example, Cl-amidine and F-amidine. In some embodiments, the PAD4 inhibitor can be specific for PAD4. In some embodiments, the PAD4 inhibitor can be a PAD family inhibitor. PAD4 inhibitors are commercially available, e.g., Cl-amidine (Catalog number 10599, CAS 913723-61-2, Cayman Chemical; Ann Arbor, Mich.) and F-amidine (Catalog number 10610; Cayman Chemica; Ann Arbor, Mich.).

As used herein, “Cl-aminidine” refers to a compound having the structure of formula I.

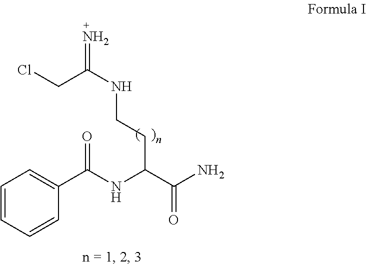

As used herein, “Fl-amidine” refers to a compound having the structure of formula II:

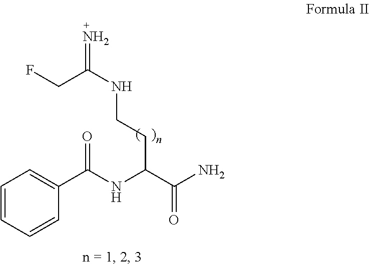

Generally, the small molecule inhibitor binds with PAD4 and inhibits a function and/or activity of PAD4. As used herein, the term “small molecules” refers to natural or synthetic molecules including, but not limited to, amino acids, peptides, peptidomimetics, polynucleotides, aptamers, nucleotide analogs, organic or inorganic compounds (i.e., including heterorganic and organometallic compounds), saccharides (e.g., mono, di, tri and polysaccharides), steroids, hormones, pharmaceutically derived drugs (e.g., synthetic or naturally occurring), lipids, derivatives of these (e.g., esters and salts of these), fragments of these, and conjugates of these. In some implementations the small molecules have a molecular weight less than about 5,000 Da, organic or inorganic compounds having a molecular weight less than about 2,500 Da, organic or inorganic compounds having a molecular weight less than about 1,000 Da, organic or inorganic compounds having a molecular weight less than about 500 Da. In some implementations the small molecule has a molecular weight of less than about 1000 Da.

In some embodiments of any one of the aspects described herein, the small molecule inhibitor is selected from the group consisting of (R)-(3-aminopiperidin-1-yl)(2-(3-bromo-1-(cyclopropylmethyl)-1H-indol-2-yl)-3-methylimidazo[1,2-a]pyridin-7-yl)methanone, (R)-(3-aminopiperidin-1-yl)(2-(1-(cyclopropylmethyl)-1H-indol-2-yl)-3-methylimidazo[1,2-a]pyridin-7-yl)methanone, (R)-(3-aminopiperidin-1-yl)(2-(1-benzyl-1H-indol-2-yl)-3-methylimidazo[1,2-a]pyridin-7-yl)methanone, (R)-(3-aminopiperidin-1-yl)(2-(1-ethyl-1H-indol-2-yl)-3-methylimidazo[1,2-a]pyridin-7-yl)methanone, (R)-(3-aminopiperidin-1-yl)(2-(1-ethyl-3-phenyl-1H-indol-2-yl)-3-methylimidazo[1,2-a]pyridin-7-yl)methanone, (R)-(3-aminopyrrolidin-1-yl)(2-(1-ethyl-3-phenyl-1H-indol-2-yl)-3-methylimidazo[1,2-a]pyridin-7-yl)methanone, (R)-(3-aminopyrrolidin-1-yl)(2-(1-ethyl-1H-indol-2-yl)-3-methylimidazo[1,2-a]pyridin-7-yl)methanone, (R)-(3-aminopyrrolidin-1-yl)(2-(1-(cyclopropylmethyl)-1H-indol-2-yl)-3-methylimidazo[1,2-a]pyridin-7-yl)methanone, (2-(1-(cyclopropylmethyl)-1H-indol-2-yl)-3-methylimidazo[1,2-a]pyridin-7-yl)(hexahydro-2H-pyrido[4,3-b][1,4]oxazin-6(5H)-yl)methanone, 10) (R)-(3-aminopiperidin-1-yl)(3-methyl-2-(1-(pyridin-4-ylmethyl)-1H-indol-2-yl)imidazo[1,2-a]pyridin-7-yl)methanone, (R)-(3-Aminopiperidin-1-yl)(3-methyl-2-(1-((tetrahydro-2H-pyran-4-yl)methyl)-1H-indol-2-yl)imidazo[1,2-a]pyridin-7-yl)methanone, (R)-(3-aminopiperidin-1-yl)(2-(1-(4-methoxybenzyl)-1H-indol-2-yl)-3-methylimidazo[1,2-a]pyridin-7-yl)methanone, (R)-(3-aminopiperidin-1-yl)(2-(1-(4-fluorobenzyl)-1H-indol-2-yl)-3-methylimidazo[1,2-a]pyridin-7-yl)methanone, (R)-4-((2-(7-(3-aminopiperidine-1-carbonyl)-3-methylimidazo[1,2-a]pyridin-2-yl)-1H-indol-1-yl)methyl)benzonitrile, (R)-(3-aminopiperidin-1-yl)(3-methyl-2-(1-(pyridin-3-ylmethyl)-1H-indol-2-yl)imidazo[1,2-a]pyridin-7-yl)methanone, (R)-(3-aminopiperidin-1-yl)(3-methyl-2-(1-(pyridin-2-ylmethyl)-1H-indol-2-yl)imidazo[1,2-a]pyridin-7-yl)methanone, (R)-(3-aminopiperidin-1-yl)(3-methyl-2-(1-(2,2,2-trifluoroethyl)-1H-indol-2-yl)imidazo[1,2-a]pyridin-7-yl)methanone, (R)-4-((2-(7-(3-aminopiperidine-1-carbonyl)-3-methylimidazo[1,2-a]pyridin-2-yl)-1H-indol-1-yl)methyl)-1-methylpyridin-2(1H)-one, (R)-(3-aminopiperidin-1-yl)(2-(3-ethylbenzo[b]thiophen-2-yl)-3-methylimidazo[1,2-a]pyridin-7-yl)methanone, (R)-(3-aminopiperidin-1-yl)(2-(1-(4-chlorobenzyl)-1H-indol-2-yl)-3-methylimidazo[1,2-a]pyridin-7-yl)methanone, (R)-(3-aminopiperidin-1-yl)(2-(1-(2-fluorobenzyl)-1H-indol-2-yl)-3-methylimidazo[1,2-a]pyridin-7-yl)methanone, (R)-(3-aminopiperidin-1-yl)(2-(1-ethyl-5-phenyl-1H-pyrrol-2-yl)-3-methylimidazo[1,2-a]pyridin-7-yl)methanone, (R)-(3-aminopiperidin-1-yl)(2-(1-(cyclopropylmethyl)-6-methoxy-1H-indol-2-yl)-3-methylimidazo [1,2-a]pyridin-7-yl)methanone, (R)-(3-aminopiperidin-1-yl)(2-(1-ethyl-1H-pyrrolo[2,3-b]pyridin-2-yl)-3-methylimidazo [1,2-a]pyridin-7-yl)methanone, (R)-(3-aminopiperidin-1-yl)(2-(1-(cyclopropylmethyl)-1H-indol-2-yl)-3,5-dimethylimidazo[1,2-a]pyridin-7-yl)methanone, (R)-(3-aminopiperidin-1-yl)(2-(1-benzyl-1H-indol-2-yl)-3,5-dimethylimidazo[1,2-a]pyridin-7-yl)methanone, 27) (R)-(3-aminopiperidin-1-yl)(3,5-dimethyl-2-(1-(pyridin-2-ylmethyl)-1H-indol-2-yl)imidazo[1,2-a]pyridin-7-yl)methanone, (R)-(3-aminopiperidin-1-yl)(2-(1-(cyclopropylmethyl)-6-fluoro-1H-indol-2-yl)-3-methylimidazo[1,2-a]pyridin-7-yl)methanone, (R)-(3-aminopiperidin-1-yl)(2-(1-(cyclopropylmethyl)-5-fluoro-1H-indol-2-yl)-3-methylimidazo[1,2-a]pyridin-7-yl)methanone, (R)-(3-aminopiperidin-1-yl)(2-(1-(cyclopropylmethyl)-7-methyl-1H-indol-2-yl)-3-methylimidazo[1 ,2-a]pyridin-7-yl)methanone, (R)-(3-aminopiperidin-1-yl)(2-(2-ethylphenyl)-3-methylimidazo[1,2-a]pyridin-7-yl)methanone, (R)-(3-aminopiperidin-1-yl)(2-(1-(cyclopropylmethyl)-1H-indol-2-yl)-3-phenylimidazo[1,2-a]pyridin-7-yl)methanone, (R)-(3-aminopiperidin-1-yl)(3-cyclopropyl-2-(1-(cyclopropylmethyl)-1H-indol-2-yl)imidazo[1,2-a]pyridin-7-yl)methanone, (R)-(3-aminopiperidin-1-yl)(2-(1-(cyclopropylmethyl)-1H-indol-2-yl)-3-(methoxymethyl)imidazo[1 ,2-a]pyridin-7-yl)methanone, (R)-(3-aminopiperidin-1-yl)(2-(1-(3-fluorobenzyl)-1H-indol-2-yl)-3-methylimidazo[1,2-a]pyridin-7-yl)methanone, (R)-(3-aminopiperidin-1-yl)(3-methyl-2-(1-(thiophen-3-ylmethyl)-1H-indol-2-yl)imidazo[1,2-a]pyridin-7-yl)methanone, (R)-(3-aminopiperidin-1-yl)(2-(1-(furan-3-ylmethyl)-1H-indol-2-yl)-3-methylimidazo[1,2-a]pyridin-7-yl)methanone, (R)-3-aminopiperidin-1-yl)(2-(1-(1-(4-fluorophenyl)ethyl)-1H-indol-2-yl)-3-methylimidazo[1,2-a]pyridin-7-yl)methanone, (R)-(3-aminopiperidin-1-yl)(2-(1-ethyl-5-fluoro-1H-indol-2-yl)-3-methylimidazo[1,2-a]pyridin-7-yl)methanone, (R)-(3-aminopiperidin-1-yl)(2-(1-(cyclopropylmethyl)-4-fluoro-1H-indol-2-yl)-3-methylimidazo[1 ,2-a]pyridin-7-yl)methanone, (R)-(3-aminopiperidin-yl)(3-methyl-2-(1-((4-methylthiazol-2-yl)methyl)-1H-indol-2-yl)imidazo[1,2-a]pyridin-7-yl)methanone, (R)-(3-aminopiperidin-1-yl)(2-(1-(3-methoxybenzyl)-1H-indol-2-yl)-3-methylimidazo[1,2-a]pyridin-7-yl)methanone, (R)-(3-aminopiperidin-1-yl)(2-(5-bromo-1-(cyclopropylmethyl)-1H-indol-2-yl)-3-methylimidazo[1,2-a]pyridin-7-yl)methanone, 44) (R)-(3-aminopiperidin-1-yl)(2-(7-chloro-1-(cyclopropylmethyl)-1H-indol-2-yl)-3-methylimidazo[1,2-a]pyridin-7-yl)methanone, (R)-(3-aminopiperidin-1-yl)(2-(1-(4-fluorobenzyl)-1H-indol-2-yl)-3-methylimidazo[1,2-a]pyridin-7-yl)methanethione, (R)-(3-aminopiperidin-1-yl)(3-methyl-2-(1-methyl-1H-indol-2-yl)imidazo[1,2-a]pyridin-7-yl)methanone, (R)-(3-aminopiperidin-1-yl)(2-(1-(cyclopropylmethyl)-7-methoxy-1H-indol-2-yl)-3-methylimidazo [1,2-a]pyridin-7-yl)methanone, (R)-(3-aminopiperidin-1-yl)(2-(1-((2,4-dimethylthiazol-5-yl)methyl)-1H-indol-2-yl)-3-methylimidazo[1,2-a]pyridin-7-yl)methanone, (R)-(3-aminopiperidin-1-yl)(3-methyl-2-(1-((2-methylthiazol-5-yl)methyl)-1H-indol-2-yl)imidazo[1,2-a]pyridin-7-yl)methanone, (R)-(3-aminopiperidin-1-yl) (2-(1-(3-methoxybenzyl)-1H-indol-2-yl)-3-methylimidazo[1,2-a]pyridin-7-yl)methanone trifluoroacetic acid salt, (R)-(3-aminopiperidin-1-yl)(2-(6-methoxy-1-(pyridin-3-ylmethyl)-1H-indol-2-yl)-3-methylimidazo[1,2-a]pyridin-7-yl)methanone, (R)-(3-aminopiperidin-1-yl)(2-(1-(cyclopropylmethyl)-6-fluoro-1H-indol-2-yl)-3-methylimidazo[1,2-a]pyridin-7-yl)methanone trifluoroacetic acid salt, (R)-(3-aminopiperidin-1-yl)(2-(5-fluoro-1-(3-methoxybenzyl)-1H-indol-2-yl)-3-methylimidazo[1,2-a]pyridin-7-yl)methanone, (R)-(3-aminopiperidin-1-yl)(2-(6-fluoro-1-(4-fluorobenzyl)-1H-indol-2-yl)-3-methylimidazo[1,2-a]pyridin-7-yl)methanone, (R)-(3-aminopiperidin-1-yl)(2-(5,6-difluoro-1-(3-methoxybenzyl)-1H-indol-2-yl)-3-methylimidazo[1,2-a]pyridin-7-yl)methanone, (R,E)-4-(dimethylamino)-N-(1-(2-(1-(4-fluorobenzyl)-1H-indol-2-yl)-3-methylimidazo[1,2-a]pyridine-7-carbonyl)piperidin-3-yl)but-2-enamide, (R)-(3-aminopiperidin-1-yl)(3-methyl-2-(1-(pyrazin-2-ylmethyl)-1H-indol-2-yl)imidazo[1,2-a]pyridin-7-yl)methanone, (R)-(3-aminopiperidin-1-yl)(3-methyl-2-(1-(pyrimidin-5-ylmethyl)-1H-indol-2-yl)imidazo[1,2-a]pyridin-7-yl)methanone trifluoroacetic acid salt, (R)-(3-aminopiperidin-1-yl)(3-methyl-2-(1-(pyridazin-3-ylmethyl)-1H-indol-2-yl)imidazo[1,2-a]pyridin-7-yl)methanone, (R)-(3-aminopiperidin-1-yl)(2-(1-isobutyl-1H-indol-2-yl)-3-methylimidazo[1,2-a]pyridin-7-yl)methanone, (R)-(3-aminopiperidin-1-yl)(2-(1-(cyclobutylmethyl)-1H-indol-2-yl)-3-methylimidazo[1,2-a]pyridin-7-yl)methanone, (R)-(3-aminopiperidin-1-yl)(2-(1-((3-fluoropyridin-2-yl)methyl)-1H-indol-2-yl)-3-methylimidazo[1,2-a]pyridin-7-yl)methanone, (R)-(3-aminopiperidin-1-yl)(2-(1-((5-methoxypyridin-2-yl)methyl)-1H-indol-2-yl)-3-methylimidazo[1,2-a]pyridin-7-yl)methanone, (R)-(3-aminopiperidin-1-yl)(2-(1-(2-methoxyethyl)-1H-indol-2-yl)-3-methylimidazo[1,2-a]pyridin-7-yl)methanone, (R)-(3-aminopiperidin-1-yl)(2-(1-(2-hydroxyethyl)-1H-indol-2-yl)-3-methylimidazo[1,2-a]pyridin-7-yl)methanone, (R)-(3-aminopiperidin-1-yl)(2-(6-methoxy-1-(pyridin-4-ylmethyl)-1H-indol-2-yl)-3-methylimidazo[1,2-a]pyridin-7-yl)methanone, (R)-(3-aminopiperidin-1-yl)(2-(1-ethyl-6-methoxy-1H-indol-2-yl)-3-methylimidazo[1,2-a]pyridin-7-yl)methanone, (R)-(3-aminopiperidin-1-yl)(2-(6-fluoro-1-(pyridin-4-ylmethyl)-1H-indol-2-yl)-3-methylimidazo[1,2-a]pyridin-7-yl)methanone, (R)-(3-aminopiperidin-1-yl)(2-(6-fluoro-1-(pyridin-3-ylmethyl)-1H-indol-2-yl)-3-methylimidazo[1,2-a]pyridin-7-yl)methanone, (R)-(3-aminopiperidin-1-yl)(2-(6-fluoro-1-(3-methoxybenzyl)-1H-indol-2-yl)-3-methylimidazo[1,2-a]pyridin-7-yl)methanone, (R)-(3-aminopiperidin-1-yl)(2-(6-fluoro-1-(4-methoxybenzyl)-1H-indol-2-yl)-3-methylimidazo[1,2-a]pyridin-7-yl)methanone, (R)-(3-aminopiperidin-1-yl)(2-(1-ethyl-6-fluoro-1H-indol-2-yl)-3-methylimidazo[1,2-a]pyridin-7-yl)methanone, (R)-(3-aminopiperidin-1-yl)(2-(6-fluoro-1-isobutyl-1H-indol-2-yl)-3-methylimidazo[1,2-a]pyridin-7-yl)methanone, (R)-(3-aminopiperidin-1-yl)(2-(5-fluoro-1-(pyridin-4-ylmethyl)-1H-indol-2-yl)-3-methylimidazo[1,2-a]pyridin-7-yl)methanone, (R)-(3-aminopiperidin-1-yl)(2-(5-fluoro-1-(4-methoxybenzyl)-1H-indol-2-yl)-3-methylimidazo[1,2-a]pyridin-7-yl)methanone, (R)-(3-aminopiperidin-1-yl)(2-(5-fluoro-1-(4-fluorobenzyl)-1H-indol-2-yl)-3-methylimidazo[1,2-a]pyridin-7-yl)methanone, (R)-(3-aminopiperidin-1-yl)(2-(7-chloro-1-(4-fluorobenzyl)-1H-indol-2-yl)-3-methylimidazo[1,2-a]pyridin-7-yl)methanone, (R)-(3-aminopiperidin-1-yl)(2-(1-(2,2-difluoroethyl)-1H-indol-2-yl)-3-methylimidazo[1,2-a]pyridin-7-yl)methanone trifluoroacetic acid salt, (R)-(3-aminopiperidin-1-yl)(2-(1-((5-fluoropyridin-2-yl)methyl)-1H-indol-2-yl)-3-methylimidazo[1,2-a]pyridin-7-yl)methanone, (R)-(3-aminopiperidin-1-yl)(2-(1-(4-fluorobenzyl)-6-methoxy-1H-indol-2-yl)-3-methylimidazo[1,2-a]pyridin-7-yl)methanone, (R)-(3-aminopiperidin-1-yl)(2-(1-(4-(hydroxymethyl)benzyl)-1H-indol-2-yl)-3-methylimidazo[1,2-a]pyridin-7-yl)methanone, (R)-(3-aminopiperidin-1-yl)(2-(1-isobutyl-6-methoxy-1H-indol-2-yl)-3-methylimidazo[1,2-a]pyridin-7-yl)methanone, (R)-(3-aminopiperidin-1-yl)(2-(1-(2,2-difluoroethyl)-6-methoxy-1H-indol-2-yl)-3-methylimidazo[1,2-a]pyridin-7-yl)methanone, (R)-(3-aminopiperidin-1-yl)(2-(5-fluoro-1-isobutyl-1H-indol-2-yl)-3-methylimidazo[1,2-a]pyridin-7-yl)methanone, (R)-(3-aminopiperidin-1-yl)(2-(1-(4-fluoro-3-methoxybenzyl)-1H-indol-2-yl)-3-methylimidazo[1,2-a]pyridin-7-yl)methanone, and (R)-(3-aminopiperidin-1-yl)(2-(1-(cyclopropylmethyl)-6-methoxy-1H-indol-2-yl)-3-(methoxymethyl)imidazo[1,2-a]pyridin-7-yl)methanone are described in US Patent Publication No. US 2020-0276206 A1 and US 20200237771 Al, the contents of which is incorporated herein by reference in its entirety. In some embodiments of any one of the aspects described herein, the inhibitor is (R)-(3-aminopiperidin-1-yl)(2-(1-(4-fluorobenzyl)-1H-indol-2-yl)-3-methylimidazo[1,2-a]pyridine-7-yl)methanone compound. The terms (R)-(3-aminopiperidin-1-yl)(2-(1-(4-fluorobenzyl)-1H-indol-2-yl)-3-methylimidazo[1,2-a]pyridine-7-yl)methanone and JBI-589 are synonymous and can be used interchangeably throughout the application.

In some embodiments of any one of the aspects described herein, the small molecule inhibitor inhibits PAD4. In some preferred embodiments, the small molecule can contact PAD4 in a covalent or non-covalent manner.

In one embodiment, the PAD4 inhibitor is JBI-589.

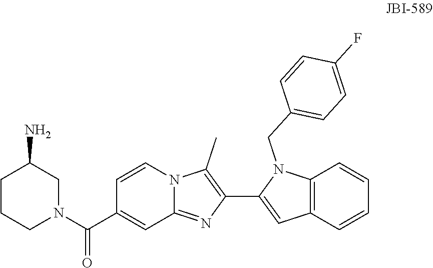 [SMILES: Cc1c(-c2cc3ccccc3n2Cc2ccc(F)cc2)nc2cc(C(=O)N3CCC[C@@H](N)C3)ccn12]

### Derivatives and Prodrugs

In various embodiments, compounds of small molecule inhibitors of PAD4 (i.e., JBI-589) include enantiomers, derivatives, prodrugs, and pharmaceutically acceptable salts thereof. The term “derivative” as used herein means any chemical, conservative substitution, or structural modification of an agent. The chemical substance can relate structurally to another, i.e., an “original” substance, which can be referred to as a “parent” compound. The derivative can improve characteristics of the agent or small molecule such as pharmacodynamics, pharmacokinetics, absorption, distribution, delivery, targeting to a specific receptor, or efficacy. A “derivative” can be made from the structurally-related parent compound in one or more steps. The general physical and chemical properties of a derivative are also similar to the parent compound.

### Left-Side Heart Failure

A subject having HFpEF, diastolic heart failure, or diastolic dysfunction can suffer from conditions such as shortness of breath including exercise induced dyspnea, paroxysmal nocturnal dyspnea and orthopnea, exercise intolerance, fatigue, elevated jugular venous pressure, and edema.

### Combination Therapies

The methods described herein can further comprise administering a second agent and/or treatment to the subject, e.g. as part of a combinatorial therapy. The additional therapy can be administered prior to, simultaneously with, or after administering the PAD4 inhibitor.

The phrase “combination therapy” as used herein means administration a PAD4 inhibitor and one or more additional therapies as part of a specific treatment regimen intended to provide a beneficial effect from the co-action of these. The beneficial effect of the combination includes, but is not limited to, pharmacokinetic or pharmacodynamic co-action resulting from the combination of therapeutic agents. Administration of these therapeutic agents in combination typically is carried out over a defined time period. The time period may be in minutes, hours, days or weeks depending upon the combination selected.

Combination therapy includes administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be done, for example, by administering to the subject a single pill having a fixed ratio of each therapeutic agent or in multiple, single pills for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered may or may not be important.

Combination therapy also can mean the administration of one or more inhibitors of PAD4 in further combination with other compounds and non-drug therapies, such as, but not limited to, surgery or radiation treatment. Where the combination therapy further comprises radiation treatment, the radiation treatment may be conducted at any suitable time so long as a beneficial effect from the co-action of the combination of the therapeutic agents and radiation treatment is achieved.

### Methods for Determining Expression Levels

In some embodiments of any of the aspects, the methods described herein are directed to determination of the expression level of PAD4 in a biological sample of a subject.

In some embodiments of any of the aspects, the methods described herein are directed to determination of the expression level of a gene product (e.g., protein and/or gene transcript such as mRNA) in a biological sample from a subject.

In one embodiment of any aspect, PAD4 expression and/or activity is inhibited by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%,or more as compared to expression and/or activity prior to administration, or a reasonable control. As used herein, “reasonable control” refers to an otherwise identical sample that is not administered a PAD4 inhibitor.

In some embodiments of any of the aspects, measurement of the level of a target and/or detection of the level or presence of a target, e.g., of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., cardiomyocytes or heart tissue from a patient diagnosed with HFpEF, diastolic heart failure, or diastolic dysfunction. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. a mRNA or polypeptide can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

In some embodiments of any of the aspects, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay.

An immunoassay is a biochemical test that measures the concentration of a substance in a biological sample, typically a fluid sample such as blood or serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments of any of the aspects, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, an adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. A dot blot immobilizes a protein sample on a defined region of a support, which is then probed with antibody and labelled secondary antibody as in Western blotting. The intensity of the signal from the detectable label in either format corresponds to the amount of enzyme present, and therefore the amount of polypeptide. Levels can be quantified, for example by densitometry.

In some embodiments of any of the aspects, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the expression levels as described herein can be determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray-based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e., each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the level of an mRNA can be measured by a quantitative sequencing technology, e.g., a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments of any of the aspects, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g., antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of any of the aspects, a detectable label can be a radiolabel including, but not limited to ³H, ¹²⁵, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments, the methods described herein relate to treating a subject having or diagnosed as having HFpEF, diastolic heart failure, or diastolic dysfunction. In some embodiments of any of the aspects, the subject is a current or former heart disease patient. In some embodiments of any of the aspects, the subject has been exposed to chemotherapy, radiation, viral infection, or certain chemicals. In some embodiments of any of the aspects, the subject has or is diagnosed as having HFpEF, diastolic heart failure, or diastolic dysfunction. Administration

In some embodiments, the methods described herein relate to treating or preventing heart failure in a subject in need thereof, the method comprising administering to the subject a PAD4 inhibitor, e.g., JBI-589. In other embodiments, the methods described herein relate to treating or preventing heart failure in a subject in need thereof, the method comprising administering to the subject a JBI-589. In one embodiment, the subject has been diagnosed as having or at risk of having heart failure, e.g., HFpEF, diastolic heart failure, or diastolic dysfunction. Subjects having HFpEF, diastolic heart failure, or diastolic dysfunction can be identified by a physician using current methods of diagnosing HFpEF, diastolic heart failure, or diastolic dysfunction. Symptoms and/or complications of HFpEF, diastolic heart failure, or diastolic dysfunction which characterize these conditions and aid in diagnosis are well known in the art and include but are not limited to, chest discomfort, decreased exercise tolerance, fatigue, shortness of breath with exertion or while at rest, and swelling in the lower extremities.

In one embodiment, the method further comprises the step, prior to administering, diagnosing the subject of having or at risk of having heart failure. In one embodiment, the method further comprises the step, prior to administering, receiving the results of an assay that diagnoses the subject of having or at risk of having heart failure.

HFpEF, diastolic heart failure, and diastolic dysfunction are diagnosed using the clinical criteria of symptoms and signs typical of heart failure. Preserved ejection fraction (EF) is defined as an EF >50% left ventricular ejection fraction (LVEF) with a left ventricle that is not dilated, relevant structural heart disease (such as concentric remodeling, left ventricular hypertrophy or left atrial enlargement), and diastolic dysfunction with increased LV filling pressures at rest, exercise, or other provocations as determined by echocardiography, invasive hemodynamic measurement or elevated levels of natriuretic peptides. One who is skilled in the art will be able to use the clinical criteria and identify a subject that is suffering from HFpEF, diastolic heart failure, or diastolic dysfunction.

Tests that may aid in a diagnosis of heart failure e.g., HFpEF, diastolic heart failure, or diastolic dysfunction include, but are not limited to, echocardiography or cardiac catheterization. A family history of HFpEF, diastolic heart failure, or diastolic dysfunction or exposure to risk factors for HFpEF, diastolic heart failure, or diastolic dysfunction (e.g. aging, anemia, atrial fibrillation, chronic kidney disease, chronic obstructive pulmonary disease (COPD), coronary artery disease, diabetes, hypertension (high blood pressure), inflammatory or autoimmune diseases, obesity, sleep apnea) can also aid in determining if a subject is likely to have HFpEF, diastolic heart failure, or diastolic dysfunction or in making a diagnosis of HFpEF, diastolic heart failure, or diastolic dysfunction.

The compositions and methods described herein can be administered to a subject having or diagnosed as having HFpEF, diastolic heart failure, or diastolic dysfunction. In some embodiments, the methods described herein comprise administering an effective amount of compositions described herein, e.g. a PAD4 inhibitor to a subject in order to alleviate a symptom of a HFpEF, diastolic heart failure, or diastolic dysfunction. As used herein, “alleviating a symptom of a HFpEF, diastolic heart failure, or diastolic dysfunction” is ameliorating any condition or symptom associated with the HFpEF, diastolic heart failure, or diastolic dysfunction. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a PAD4 inhibitor needed to alleviate at least one or more symptom of the disease or disorder, and relates to a sufficient amount of pharmacological composition to provide the desired effect. The term “therapeutically effective amount” therefore refers to an amount of a PAD4 inhibitor that is sufficient to provide a particular anti-left ventricle thickening effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a PAD4 inhibitor, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for PAD4 expression or activity, among others. The dosage can be determined by a physician and adjusted, as necessary, to suit observed effects of the treatment.

In some embodiments, the technology described herein relates to a pharmaceutical composition comprising a PAD4 inhibitor as described herein, and optionally a pharmaceutically acceptable carrier. In some embodiments, the active ingredients of the pharmaceutical composition comprise a PAD4 inhibitor as described herein. In some embodiments, the active ingredients of the pharmaceutical composition consist essentially of a PAD4 inhibitor as described herein. In some embodiments, the active ingredients of the pharmaceutical composition consist of a PAD4 inhibitor as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a PAD4 inhibitor as described herein.

In some embodiments, the PAD4 inhibitor can be used at a dose between 0.1 mg/kg-0.2 mg/kg, between 0.1 mg/kg-0.3 mg/kg, between 0.1 mg/kg-0.4 mg/kg, between 0.1 mg/kg-0.5 mg/kg, between 0.1 mg/kg-0.6 mg/kg, between 0.1 mg/kg-0.7 mg/kg, between 0.1 mg/kg-0.8 mg/kg, between 0.1 mg/kg-0.9 mg/kg, between 0.1 mg/kg-1.0 mg/kg, between 0.1 mg/kg-1.1 mg/kg, between 0.1 mg/kg-1.2 mg/kg, between 0.1 mg/kg-1.3 mg/kg, between 0.1 mg/kg-1.4 mg/kg, between 0.1 mg/kg-1.5 mg/kg, between 0.1 mg/kg-1.6 mg/kg, between 0.1 mg/kg-1.7 mg/kg, between 0.1 mg/kg-1.8 mg/kg, between 0.1 mg/kg-1.9 mg/kg, between 0.1 mg/kg-2.0 mg/kg, between 0.1 mg/kg-2.1 mg/kg, between 0.1 mg/kg-2.2 mg/kg, between 0.1 mg/kg-2.3 mg/kg, between 0.1 mg/kg-2.4 mg/kg, between 0.1 mg/kg-2.5 mg/kg, between 0.1 mg/kg-2.6 mg/kg, between 0.1 mg/kg-2.7 mg/kg, between 0.1 mg/kg-2.8 mg/kg, between 0.1 mg/kg-2.9 mg/kg, between 0.1 mg/kg-3.0 mg/kg, between 0.1 mg/kg-3.1 mg/kg, 0.1 mg/kg-3.2 mg/kg, between 0.1 mg/kg-3.3 mg/kg, between 0.1 mg/kg-3.4 mg/kg, between 0.1 mg/kg-3.5 mg/kg, between 0.1 mg/kg-3.6 mg/kg, between 0.1 mg/kg-3.7 mg/kg, between 0.1 mg/kg-3.8 mg/kg, between 0.1 mg/kg-3.9 mg/kg, between 0.1 mg/kg-4.Omg/kg, between 0.1 mg/kg-4.1 mg/kg, between 0.1 mg/kg-4.2 mg/kg, between 0.1 mg/kg-4.3 mg/kg, between 0.1 mg/kg-4.4 mg/kg, between 0.1 mg/kg-4.5 mg/kg, between 0.1 mg/kg-4.6 mg/kg, between 0.1 mg/kg-4.7 mg/kg, between 0.1 mg/kg-4.8 mg/kg, between 0.1 mg/kg-4.9 mg/kg, between 0.1 mg/kg-5.Omg/kg, between 0.1 mg/kg-5.1 mg/kg, between 0.1 mg/kg-5.2 mg/kg, between 0.1 mg/kg-5.3 mg/kg, between 0.1 mg/kg-5.4 mg/kg, between 0.1 mg/kg-5.5 mg/kg, between 0.1 mg/kg-5.6 mg/kg, between 0.1 mg/kg-5.7 mg/kg, between 0.1 mg/kg-5.8 mg/kg, between 0.1 mg/kg-5.9 mg/kg, between 0.1 mg/kg-6.Omg/kg, between 0.1 mg/kg-6.1 mg/kg, 0.1 mg/kg-6.2 mg/kg, between 0.1 mg/kg-6.3 mg/kg, between 0.1 mg/kg-6.4 mg/kg, between 0.1 mg/kg-6.5 mg/kg, between 0.1 mg/kg-6.6 mg/kg, between 0.1 mg/kg-6.7 mg/kg, between 0.1 mg/kg-6.8 mg/kg, between 0.1 mg/kg-6.9 mg/kg, between 0.1 mg/kg-7.Omg/kg, between 0.1 mg/kg-7.1 mg/kg, between 0.1 mg/kg-7.2 mg/kg, between 0.1 mg/kg-7.3 mg/kg, between 0.1 mg/kg-7.4 mg/kg, between 0.1 mg/kg-7.5 mg/kg, between 0.1 mg/kg-7.6 mg/kg, between 0.1 mg/kg-7.7 mg/kg, between 0.1 mg/kg-7.8 mg/kg, between 0.1 mg/kg-7.9 mg/kg, between 0.1 mg/kg-8.Omg/kg, between 0.1 mg/kg-8.1 mg/kg, between 0.1 mg/kg-8.2 mg/kg, between 0.1 mg/kg-8.3 mg/kg, between 0.1 mg/kg-8.4 mg/kg, between 0.1 mg/kg-8.5 mg/kg, between 0.1 mg/kg-8.6 mg/kg, between 0.1 mg/kg-8.7 mg/kg, between 0.1 mg/kg-8.8 mg/kg, between 0.1 mg/kg-8.9 mg/kg, between 0.1 mg/kg-9.Omg/kg, between 0.1 mg/kg-9.1 mg/kg, between 0.1 mg/kg-9.2 mg/kg, between 0.1 mg/kg-9.3 mg/kg, between 0.1 mg/kg-9.4 mg/kg, between 0.1 mg/kg-9.5 mg/kg, between 0.1 mg/kg-9.6 mg/kg, between 0.1 mg/kg-9.7 mg/kg, between 0.1 mg/kg-9.8 mg/kg, between 0.1 mg/kg-9.9 mg/kg, between 0.1 mg/kg-10.Omg/kg, between 0.1 mg/kg-20.Omg/kg, between 0.1 mg/kg-30.Omg/kg, between 0.1 mg/kg-40.Omg/kg, between 0.1 mg/kg-50.Omg/kg, between 0.1 mg/kg-60.Omg/kg, between 0.1 mg/kg-70.Omg/kg, between 0.1 mg/kg-80.Omg/kg, between 0.1 mg/kg-90.Omg/kg, between 0.1 mg/kg-100 mg/kg, or more.

In some embodiments, the pharmaceutical composition comprising a PAD4 inhibitor as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a PAD4 inhibitor as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a PAD4 inhibitor as disclosed herein can also be incorporated into the parenteral dosage forms of the disclosure, including conventional and controlled-release parenteral dosage forms.

Another aspect described herein is a composition for the treatment or prevention of heart failure comprising a PAD4 inhibitor and a pharmaceutically acceptable carrier.

Another aspect described herein is a composition for treating or preventing heart failure, the composition comprising a PAD4 inhibitor, wherein the PAD4 inhibitor is JBI-589.

Another aspect of described herein is a use composition comprising a PAD4 inhibitor for the treatment of prevention of heart failure, wherein the PAD4 inhibitor is JBI-589.

Pharmaceutical compositions comprising a PAD4 inhibitor can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the PAD4 inhibitor can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif USA)), or a combination thereof to provide the desired release profile in varying proportions.

In some embodiments of any of the aspects, the PAD4 inhibitor described herein is administered as a monotherapy, e.g., another treatment for the HFpEF, diastolic heart failure, or diastolic dysfunction is not administered to the subject.

In some embodiments of any of the aspects, the methods described herein can further comprise administering a second agent and/or treatment to the subject, e.g., as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising a PAD4 inhibitor as described herein can be administered to a patient once. In certain embodiments, an effective dose of a composition comprising a PAD4 inhibitor can be administered to a patient repeatedly. For systemic administration, subjects can be administered a therapeutic amount of a composition comprising a PAD4 inhibitor, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g., HFpEF or diastolic heart failure by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to a PAD4 inhibitor. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a PAD4 inhibitor can be administered over a period of time, such as over a 5 minute, 10 minute, 15 minute, 20 minute, or 25-minute period.

The dosage ranges for the administration of a PAD4 inhibitor, according to the methods described herein depend upon, for example, the form of the PAD4 inhibitor, its potency, and the extent to which symptoms, markers, or indicators of a condition described herein are desired to be reduced, for example the percentage reduction desired for HFpEF or the extent to which, for example, the stiffness of the left ventricle is reduced, are desired to be induced. The dosage should not be so large as to cause adverse side effects, such as immune suppression. Generally, the dosage will vary with the age, condition, and sex of the patient and can be determined by one of skill in the art. The dosage can also be adjusted by the individual physician in the event of any complication.

The efficacy of a PAD4 inhibitor in, e.g., the treatment of HFpEF, diastolic heart failure, or diastolic dysfunction as described herein, can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., ejection fraction or stiffness of the left ventricle. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g., reduced stiffening of the left ventricle). It is well within the ability of one skilled in the art to monitor efficacy of administration and/or treatment by measuring any one of such parameters, or any combination of parameters. Efficacy can be assessed in animal models of a condition described herein, for example treatment of HFpEF, diastolic heart failure, or diastolic dysfunction. When using an experimental animal model, efficacy of treatment is evidenced when a statistically significant change in a marker is observed, e.g., stiffness of the left ventricle.

In vitro and animal model assays are provided herein which allow the assessment of a given dose of a PAD4 inhibitor. By way of non-limiting example, the effects of a dose of a PAD4 inhibitor can be assessed by stiffness of the left ventricle or ejection fraction. A non-limiting example of a protocol for such an assay is as follows: Mice received JBI-589 via oral gavage. Briefly, after grasping the mice holding them in an upright position and monitoring their breathing patterns, the feeding tube was inserted to the right or the left of midline to avoid the chance of the mouse biting the tube. If little to no resistance was felt, JBI-589 was given in suspension formulation prepared using Tween-80 and 0.5% methyl cellulose at a dose of 10 mg/kg. Animals were monitored after dosing to make sure breathing remained within normal limits.

In one respect, the present invention relates to the herein described method thereof, as essential to the technology, yet open to the inclusion of unspecified elements, essential or not (“comprising”). In some embodiments of any of the aspects, other elements to be included in the description of the method are limited to those that do not materially affect the basic and novel characteristic(s) of the technology (e.g., the method thereof “consists essentially of” the elements described herein). This applies equally to steps within a described method as well as compositions and components therein. In other embodiments of any of the aspects, the methods, described herein are intended to be exclusive of any element not deemed an essential element to the method (e.g., the method thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of HFpEF, diastolic heart failure, or diastolic dysfunction. A subject can be male or female.

As defined herein, ejection fraction is the volumetric fraction (or portion of the total) of fluid (usually blood) ejected from a chamber (usually the heart) with each contraction (or heartbeat). It can refer to the cardiac atrium, ventricle, gall bladder, or leg veins, although if unspecified it usually refers to the left ventricle of the heart. EF is widely used as a measure of the pumping efficiency of the heart and is used to classify heart failure types. It is also used as an indicator of the severity of heart failure, although it has recognized limitations.

The EF of the left heart, known as the left ventricular ejection fraction (LVEF), is calculated by dividing the volume of blood pumped from the left ventricle per beat (stroke volume) by the volume of blood collected in the left ventricle at the end of diastolic filling (end-diastolic volume). LVEF is an indicator of the effectiveness of pumping into the systemic circulation. The EF of the right heart, or right ventricular ejection fraction (RVEF), is a measure of the efficiency of pumping into the pulmonary circulation. A heart which cannot pump sufficient blood to meet the body's requirements (i.e., heart failure) will often, but not invariably, have a reduced ventricular ejection fraction. Throughout the entirety of the application, reference to “ejection fraction” is in reference to the left ventricular ejection fraction.

A subject can be one who has been previously diagnosed with or identified as suffering from or having a condition in need of treatment (e.g. HFpEF, diastolic heart failure, or diastolic dysfunction) or one or more complications related to such a condition, and optionally, have already undergone treatment for HFpEF, diastolic heart failure, or diastolic dysfunction or the one or more complications related to HFpEF, diastolic heart failure, or diastolic dysfunction. Alternatively, a subject can also be one who has not been previously diagnosed as having HFpEF, diastolic heart failure, or diastolic dysfunction or one or more complications related to HFpEF, diastolic heart failure, or diastolic dysfunction. For example, a subject can be one who exhibits one or more risk factors for HFpEF, diastolic heart failure, or diastolic dysfunction or one or more complications related to HFpEF, diastolic heart failure, or diastolic dysfunction or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect, e.g., PAD4, of the full-length polypeptide, e.g., PAD4 of SEQ ID NO: 3, e.g. as measured by flow cytometry. Conservative substitution variants that maintain the activity of wildtype PAD4 will include a conservative substitution as defined herein. The identification of amino acids most likely to be tolerant of conservative substitution while maintaining at least 50% of the activity of the wildtype is guided by, for example, sequence alignment with PAD4 homologs or paralogs from other species. Amino acids that are identical between PAD4 homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of PAD4 as described herein. Further discussion of the structure of PAD4 can be found, e.g., in U.S. Pat. No. 11,426,412 and Zhu et al. Pharmaceutics 2022, 14, 2414. which is incorporated by reference herein in its entirety.

In some embodiments, a polypeptide, e.g., a PAD4 polypeptide, can be a variant of a sequence described herein, e.g. a variant of a PAD4 polypeptide comprising the sequence of SEQ ID NO: 3. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., SEQ ID NO. 3, at least 50% as well as wildtype PAD4. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wildtype PAD4, e.g., 110%, 125%, 150%, 175%, 200%, 500%, 1000% or more.

One method of identifying amino acid residues which can be substituted is to align, for example, human PAD4 to a PAD4 homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g., SEQ ID NOs 2-3 or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g., BLASTp or BLASTn (available on the world wide web at blast.ncbi.nlm.nih.gov), with default parameters set.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., PAD4 activity and specificity of a native or reference polypeptide is retained.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Typically conservative substitutions for one another also include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide, e.g., a PAD4 polypeptide can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., a PAD4 polypeptide, as described herein can comprise at least one peptide bond replacement. A PAD4 polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., a PAD4 polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, a PAD4 polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. a PAD4 polypeptide, can be modified, e.g. by addition of a moiety to one or more of the amino acids that together comprise the peptide. In some embodiments, a polypeptide as described herein can comprise one or more moiety molecules, e.g., 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one or more more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g., PEG, albumin, or other fusion partners (e.g. Fc fragment of an immunoglobin).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., PAD4 genomic DNA or cDNA. Suitable RNA can include, e.g., PAD4 mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

“Marker” in the context of the present invention refers to an expression product, e.g., nucleic acid or polypeptide which is differentially present in a sample taken from subjects having having HFpEF, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the PAD4 sequence described herein is exogenous. In some embodiments of any of the aspects, the PAD4 sequence described herein is ectopic. In some embodiments of any of the aspects, the PAD4 sequence described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid encoding a polypeptide as described herein (e.g. a PAD4 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., HFpEF or diastolic heart failure. The term “treating” includes reducing or alleviating at least one adverse effect or symptom of a condition, disease or disorder associated with HFpEF or diastolic heart failure. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to chest discomfort, decreased exercise tolerance, fatigue, shortness of breath with exertion or while at rest, and swelling in the lower extremities. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc.), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

The inventions described herein can further be described in the following numbered paragraphs.
1. A method of treating or preventing heart failure comprising administering a PAD4 inhibitor to a subject in need thereof.
2. The method of paragraph 1, wherein the subject in need thereof has been diagnosed with having heart failure.
3. The method of any of the preceding paragraphs, wherein the subject in need thereof has been diagnosed with being at risk of having heart failure.
4. The method of any of the preceding paragraphs, wherein the PAD4 inhibitor is an antibody reagent, an inhibitory nucleic acid, or a small molecule.
5. The method of any of the preceding paragraphs, wherein the PAD4 inhibitor inhibits PAD4 expression and/or activity.
6. The method of any of the preceding paragraphs, wherein PAD4 expression and/or activity is inhibited by at least 10%, 20%, 30%, 40%, 50%, 60%, 70%, 80%, 90%, 99% or more as compared to expression and/or activity prior to administration.
7. The method of any of the preceding paragraphs, wherein the small molecule is JBI-589:

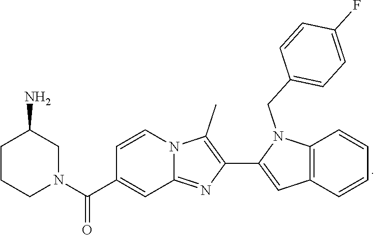 [SMILES: Cc1c(-c2cc3ccccc3n2Cc2ccc(F)cc2)nc2cc(C(=O)N3CCC[C@@H](N)C3)ccn12]

8. The method of any of the preceding paragraphs, wherein the administering is selected from the group comprising topically, intravascularly, intravenously, intraarterially, intratumorally, intramuscularly, subcutaneously, intraperitoneally, intranasally, or orally.
9. The method of any of the preceding paragraphs, wherein the heart failure comprises diastolic dysfunction, diastolic heart failure, and heart failure with preserved ejection fraction (HFpEF).
10. The method of any of the preceding paragraphs, wherein the heart failure is HFpEF.
11. The method of any of the preceding paragraphs, wherein the HFpEF arises as a result of autoimmune diseases, Diabetes Mellitus, hypertension, aging, and obesity.
12. The method of any of the preceding paragraphs, further comprising the step, prior to administering, diagnosing the subject of having or at risk of having heart failure.
13. The method of any of the preceding paragraphs, further comprising the step, prior to administering, receiving the results of an assay that diagnoses the subject of having or at risk of having heart failure.
14. The method of any of the preceding paragraphs, wherein the subject is a mammal.
15. The method of any of the preceding paragraphs, wherein the mammal is human.
16. A method of treating or preventing heart failure, the method comprising administering a PAD4 inhibitor to a subject in need thereof, wherein the PAD4 inhibitor is JBI-589.
17. A composition for the treatment or prevention of heart failure comprising a PAD4 inhibitor and a pharmaceutically acceptable carrier.
18. The composition of paragraph 17, wherein the PAD4 inhibitor is an antibody reagent, an inhibitory nucleic acid, or a small molecule.
19. The composition of any of the preceding paragraphs, wherein the PAD4 inhibitor is JBI-589:

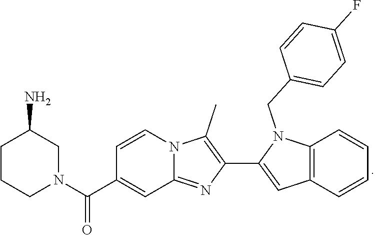 [SMILES: Cc1c(-c2cc3ccccc3n2Cc2ccc(F)cc2)nc2cc(C(=O)N3CCC[C@@H](N)C3)ccn12]

20. The composition of any of the preceding paragraphs, wherein the heart failure comprises diastolic dysfunction, diastolic heart failure, and HFpEF.
21. The composition of any of the preceding paragraphs, wherein the heart failure is HFpEF.
22. The composition of any of the preceding paragraphs, wherein the HFpEF arises as a result of autoimmune diseases, Diabetes Mellitus, hypertension, aging, and obesity.
23. The composition of any of the preceding paragraphs, wherein the subject is a mammal.
24. The composition of any of the preceding paragraphs, wherein the mammal is human.
25. A composition for treating or preventing heart failure, the composition comprising a PAD4 inhibitor, wherein the PAD4 inhibitor is JBI-589.
26. Use composition comprising a PAD4 inhibitor for the treatment of prevention of heart failure, wherein the PAD4 inhibitor is JBI-589.

### EXAMPLES

### Example 1

Background: Rheumatoid arthritis (RA) is associated with an increased incidence of Heart Failure with preserved ejection fraction (HFpEF).

Objective: Data provided herein evaluate the role of Peptidylarginine deiminase 4 (PAD4) mediated neutrophil activation in myocardial remodeling and function in RA.

Methods: Collagen induced arthritis (CIA) model in DBA/1 mice was used to generate RA. Flow cytometry, immunohistochemistry, echocardiography and histological analysis using Masson's trichrome and picrosirius Red staining was performed to determine left ventricular morphology and function. Enzymatic activity of PAD4 was inhibited using a novel oral inhibitor (JBI-589) for 26 consecutive days after onset of clinical arthritis.

Results: A significant predisposition of mice was demonstrated to be subjected to CIA to develop hypertrophic cardiac remodeling with diastolic dysfunction and preserved ejection fraction (HFpEF). Neutrophil activation and infiltration of activated neutrophils propelled by endothelial activation as well as a pro-fibrotic shift with increased deposition of Collagen I (Coll) was a key feature of affected myocardium. Inhibition of PAD4 preserved LV function, reduced Coll deposition and resulted in reduced neutrophil trafficking and activation.

Conclusion: PAD4 mediated neutrophil activation was identified and diapedesis as key mechanism in HFpEF development in RA.

### Introduction

Heart failure with preserved ejection fraction (HFpEF) is a heterogenous multiorgan systemic disorder and a critical public health problem increasing in prevalence. Overt volume overload due to left ventricular diastolic dysfunction requiring repeated hospitalisations and the lack of effective therapies culminates in reduced quality of life and poor prognosis for affected patients (1, 2) Numerous comorbidities often harboured in HFpEF patients, interconnected or alone, are suspected to contribute to the biological basis of HFpEF development. (15) What it is, that links comorbidity burden with detrimental myocardial remodelling however is unknown. Chronic comorbidity-induced systemic inflammation is likely to be involved in HFpEF development and studies have shown, that patients with rheumatoid arthritis (RA), a prototypic chronic systemic inflammatory disease are at an increased risk of developing HFpEF independent of traditional cardiovascular risk factors. (3-9) However, current anti-inflammatory RA therapies although improving RA disease severity seem to have little to no cardioprotective effect. (10-14) This facilitates the need for a better understanding of HFpEF pathophysiology in RA and the need for better targeted therapies.

Myocardial tissue stiffness caused by either cardiomyocyte stiffness, tissue fibrosis or both is highly probable to be a key player in HFpEF pathophysiology.(10, 16, 17) Correspondingly, interstitial fibrosis has been shown to be associated with a worse prognosis in HFpEF patients and two recent randomized trials (PARAGON-HF, TOPCAT) report, altered expressions of biomarkers regulating collagen homeostasis. (17-19)(20, 21). Infiltration by immune cells has been proposed as one potential mechanism driving this profibrotic shift. (22-25) Activation of neutrophils via Peptidylarginine deiminase 4 (PAD4) is comprehensively involved in RA pathophysiology. (26-28) Suchlike activated neutrophils promote fibrosis through a variety of mechanisms among witch the release of neutrophil extracellular traps (NETs) protrudes.(29, 30) After decondensation of chromatin dependent histones catalysed by PAD4, NETs are released, mounted with cytosolic histones and peptides imposing a direct cytotoxic effect on tissues wielding organ damage.(31, 32)(38-40) It is known, that neutrophils of RA patients display an increased propensity for neutrophil activation and spontaneous NET formation.(33) Interestingly, several comorbidities often harboured in HFpEF patients including aging, cardiometabolic stress, and chronic hypertension have increasingly been connected to amplified neutrophil activation.(34-40) On top of that, neutrophils through PAD4/NETs have been shown to contribute to organ fibrosis and organ function decline in aging.(41-44) Conversely, in a murine hypertension-induced HFpEF model depletion of neutrophils reduced cardiac hypertrophy and dysfunction.(45-49) Phenotypes boasting secondary myocardial fibrosis in both, a model of pressure-overload and a model of aging depend heavily on PAD4 expression. (41) Together with it being an important contributor to RA development this suggests PAD4 as possible target to alter myocardial pro-fibrotic remodelling and preserve heart function in RA.(50) CIA is a viable model to study development of HFpEF in RA and in unrevealing the underlying pathophysiology could add to the understanding of its pathobiology.

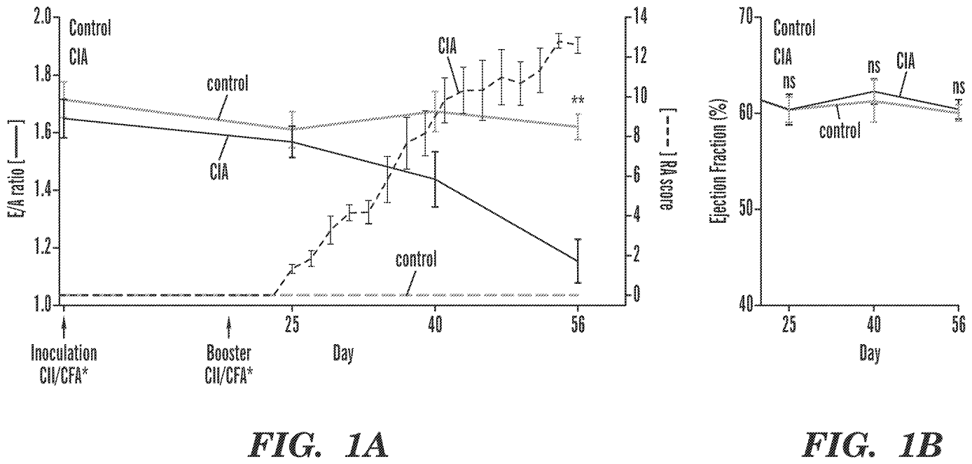

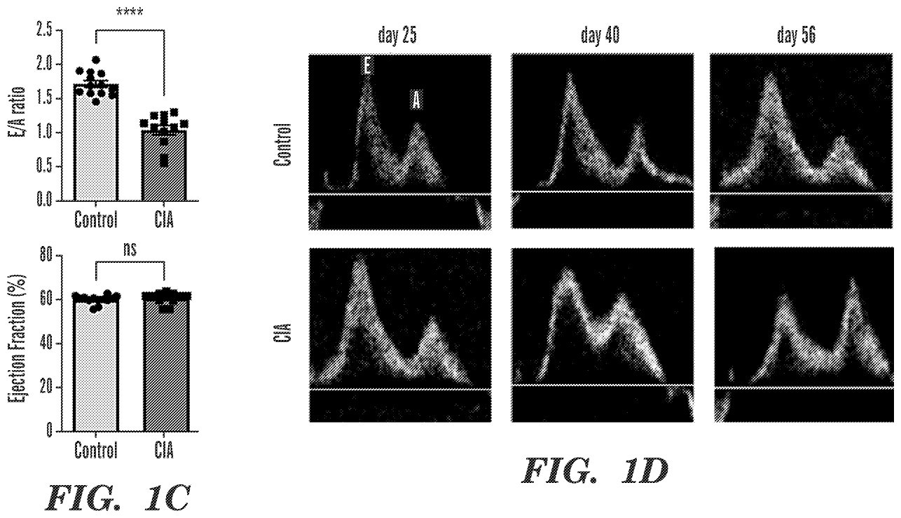

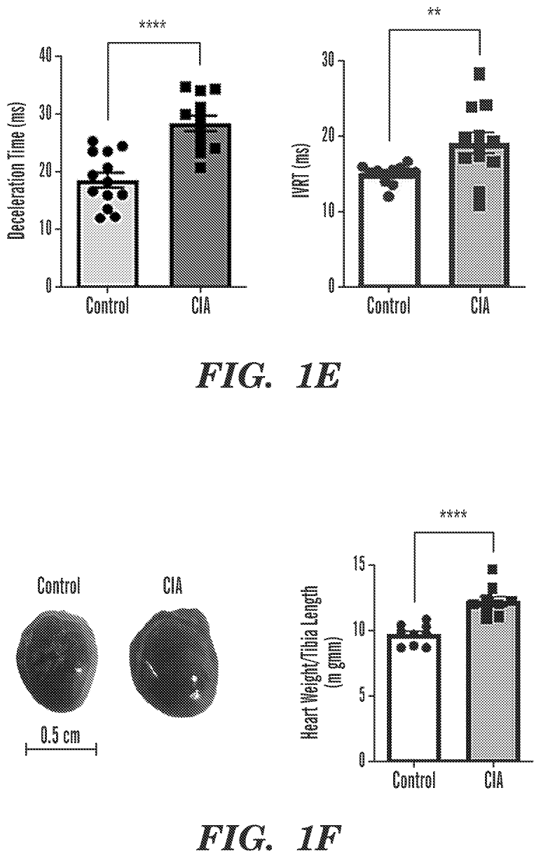

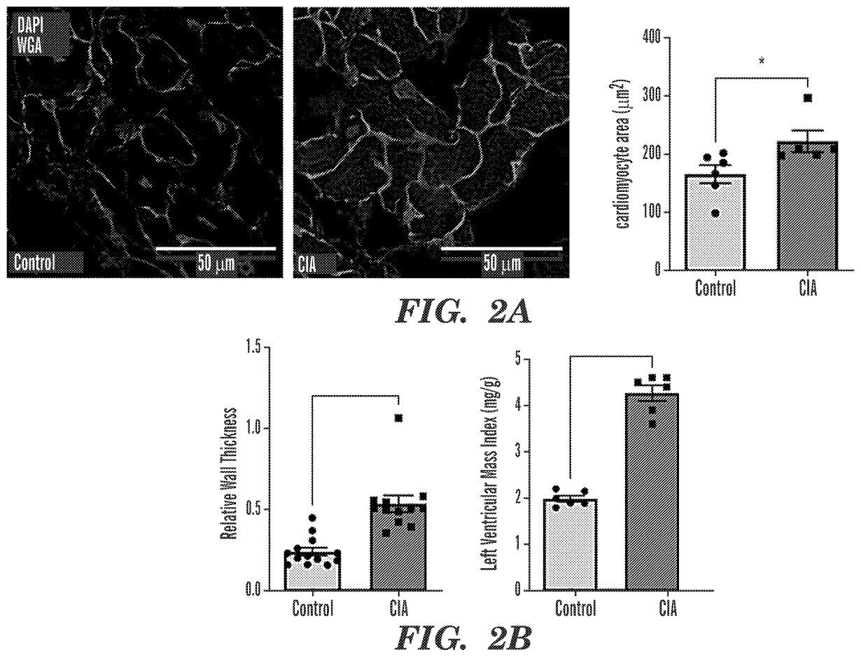

FIG. 1: Mice with Collagen Induced Arthritis (CIA) develop diastolic dysfunction and myocardial hypertrophy.

FIG. 1A: Timecourse illustrating the ratio of early (E) to late (A) diastolic filling velocities across the mitral valve as measured by pulsed wave doppler (E/A ratio; left Y axis; -). Clinical severity of rheumatoid arthritis (RA Score; right y axis; ---) in DBA/1J mice with CIA and a healthy control respectively. (n=6) Arrows indicate time point of inoculation/booster with type II collagen and (*)complete/incomplete Freud's adjuvant.’ FIG. 1B: Left ventricular ejection fraction in DBA/1J mice with CIA and a healthy control respectively, demonstrated over the course of time. (n=6)

FIG. 1C: E/A ratio and ejection fraction (EF) as assessed by echocardiography in mice with CIA and healthy control at day 56. (n=12)

FIG. 1D: Representative flow patterns, acquired using pulsed-wave Doppler echocardiography, depicting the velocities over the mitral valve at indicated timepoints in mice with CIA and healthy control respectively.

FIG. 1E: Deceleration of time reflecting the amount of time needed to equalize the pressure difference between the left atrium (LA) and the left ventricle (LV) or the time interval from the peak of the E-wave to its projected baseline and isovolumetric relaxation time (IVRT) a marker for myocardial relaxation measuring the time for crossover between the LA and LV pressures as assessed by echocardiography in arthritic CIA mice and healthy controls at day 56. (n=12)

FIG. 1F: Heart-weight normalized to tibia-length in CIA mice and healthy control group after 56 days. (n=9)

Data are mean±SEM. *P<0.05, **P<0.01, ***P<0.001; Unpaired Student's T test (IVRT: Mann-Whitney U-test).

Conclusion: In a mouse model of inflammatory arthritis, mice develop myocardial stiffness and hypertrophy with impaired diastolic filling resulting in a disruption of the cardiac cycle and heart failure with preserved ejection fraction.

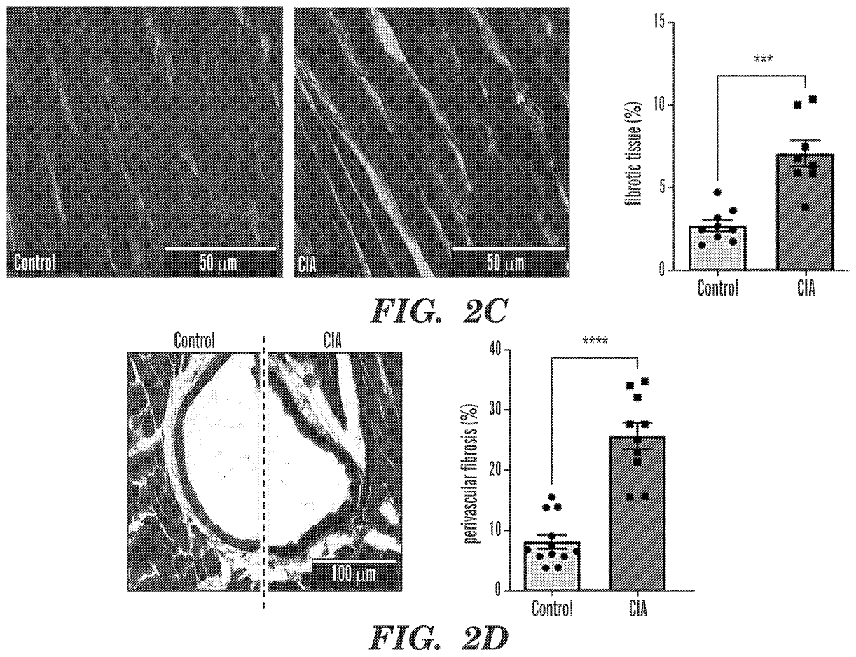

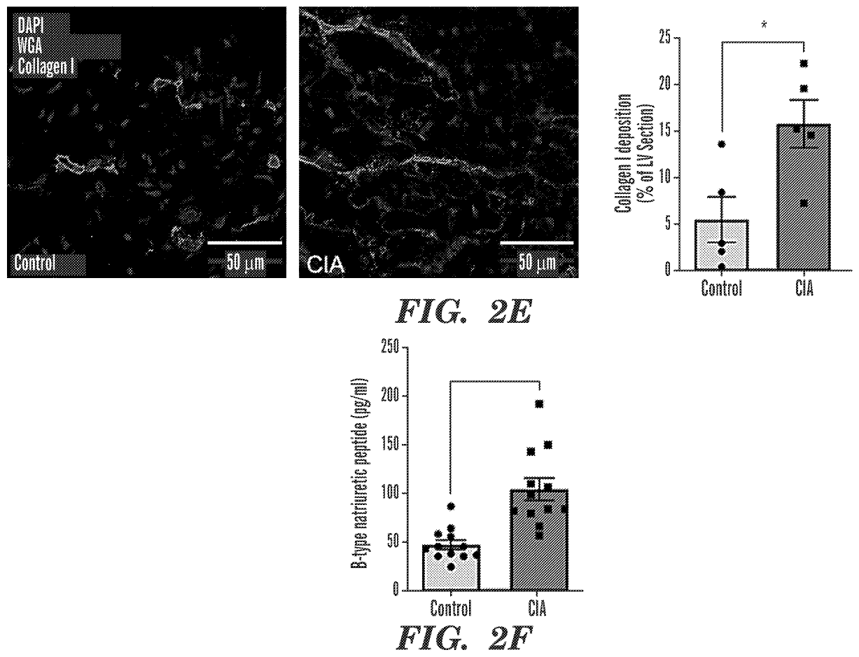

FIG. 2: Left ventricular remodeling and fibrosis in mice with CIA drive development of clinically relevant diastolic dysfunction similar to Heart Failure with Preserved Ejection Fraction (HFpEF).

FIG. 2A: Representative images of LV sections stained with Wheat germ agglutinin (WGA) for cell membrane and DAPI staining of DNA to illustrate Cardiomyocyte cell size defined by cross section area. (n=6)

FIG. 2B: Left ventricular mass (LVM) a parameter estimating total LV weight was measured using echocardiography. LVM was calculated as difference between the epicardium delimited volume and the LV chamber volume multiplied by an estimate of myocardial densityin. Diastolic LV posterior wall thickness (LVPWd) was measured at end diastole as a measure of LV geometry and expansion. (n=12)

FIG. 2C: Representative LV section images of Massom Trichrom staining for fibrotic tissue (blue) in a mouse with CIA and a healthy control respectively. Comparative analysis of total area of fibrotic tissue in LV sections. (n=9/8)

FIG. 2D: Representative images of Massom Trichrom staining with perivascular fibrosis and quantitative analysis. (For each mouse 5 peripheral vessels chosen at random were quantified and their average was used for comparative analysis; n=10)

FIG. 2E: Quantification of Collagen I (red) and vasculature (CD31+ cells) in LV. Representative images are shown. (n=5)

FIG. 2F: Plasma levels of Brain natriuretic peptide, a hormone produced by the body when the heart is enlarged, as measured by ELISA on day 56 in mice with CIA and a healthy control group. (n=12)

Data are mean±SEM. *P<0.05, **P<0.01, ***P<0.001; Mann-Whitney U-test (F: Student's t test).

Conclusion: The increase in heart weight and resulting concentric hypertrophy enlarging the heart and fueling diastolic dysfunction in mice with CIA is most likely based on increased heart fibrosis with collagen I deposition and increased cardiomyocyte cell size.

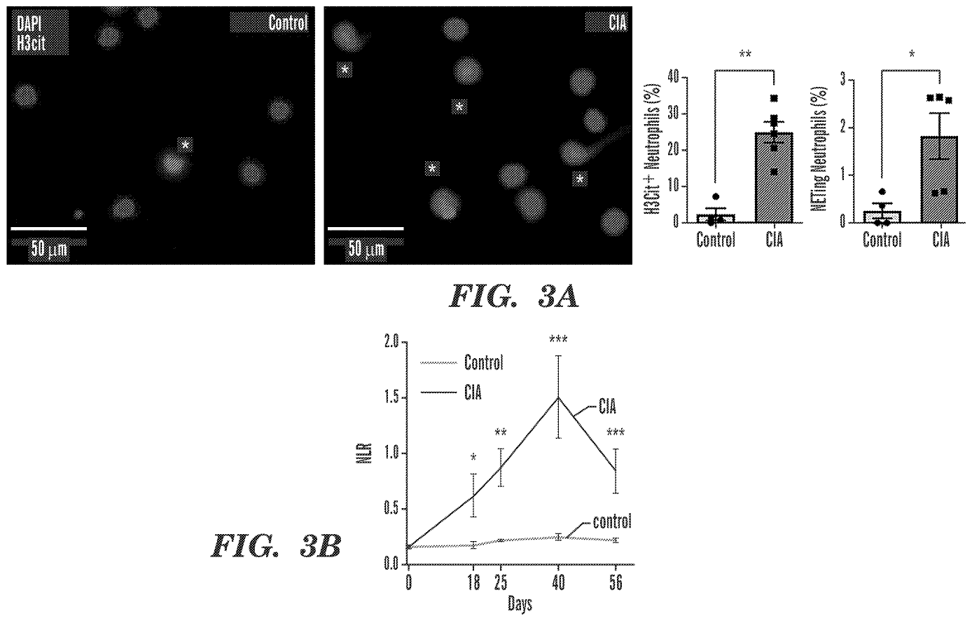

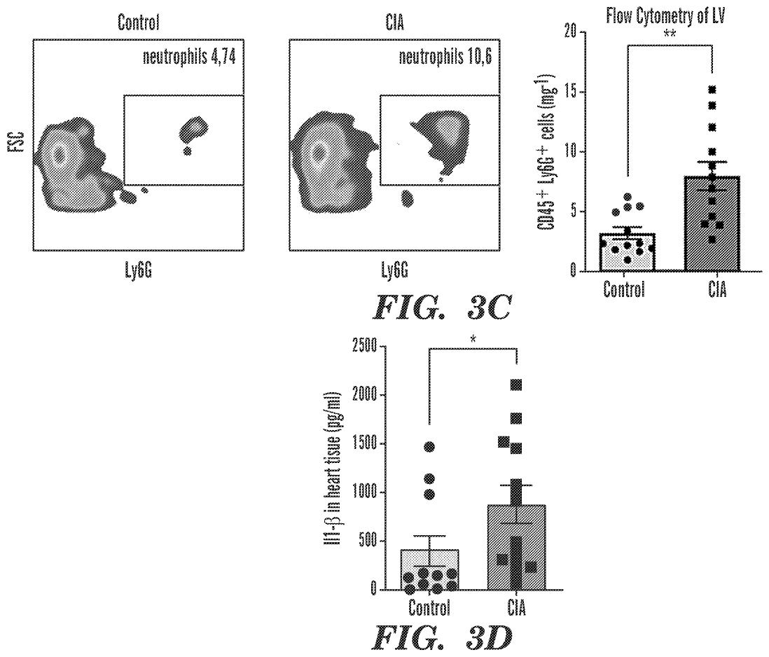

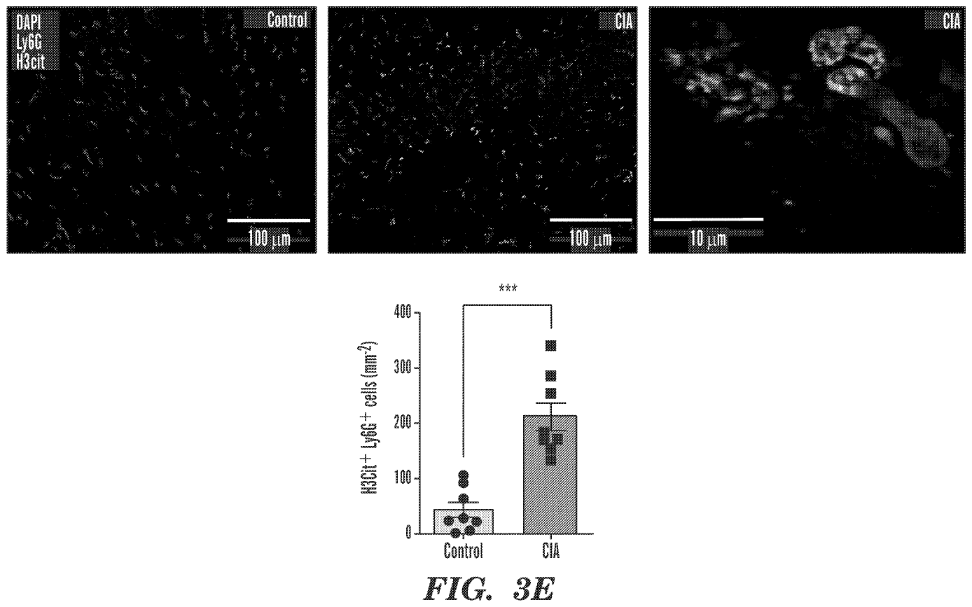

FIG. 3: Neutrophils are activated both in the circulation and in myocardium of mice with RA.

FIG. 3A: Representative immunofluorescence images of isolated neutrophils from CIA- or healthy control mice. Neutrophils from CIA mice are preactivated (H3Cit+ pink) and show a propensity for spontaneous NET formation. Representative microscopic picture of isolated neutrophils are shown. Healthy control (left) and CIA (right). *=activated H3Cit+neutrophil. (n=6)

FIG. 3B: Graph, illustrating as a function of time the neutrophil-to-lymphocyte ratio (NLR) a marker of hyperinflammatory response, measured in peripheral blood in DBA/1J mice with CIA and a healthy control respectively. (n=6)

FIG. 3C: Gating and quantification of infiltrating CD45+ and LY6G+cells using Flow Cytometry (FACS) in healthy control and CIA myocardial tissue. (n=14)

FIG. 3D: Quantitative comparison of tissue levels of Interleukin 1beta an inflammatory cytokine mediating fibrosis measured using ELISA. (n=9)

FIG. 3E: Representative LV sections of CIA mice and healthy control mice stained for DAPI+ (blue) Ly6G+ (red) and H3Cit+ (green) cells as well as a representative image of an activated neutrophil in the right panel with co-staining of Ly6G+ (red) and H3Cit+(green). Comparative analysis of total number of Ly6G+ (red) and H3cit+ (green) cells in LV heart sections of CIA mice and healthy controls. (n=8)

Data are mean±SEM. *P<0.05, **P<0.01, ***P<0.001; Mann-Whitney U-test (C,E: Student's t test).

Conclusion: Neutrophils with their pro-fibrotic properties are hyperactivated in mice with CIA and can be found in increased numbers in the myocardium of these mice when compared to healthy controls.

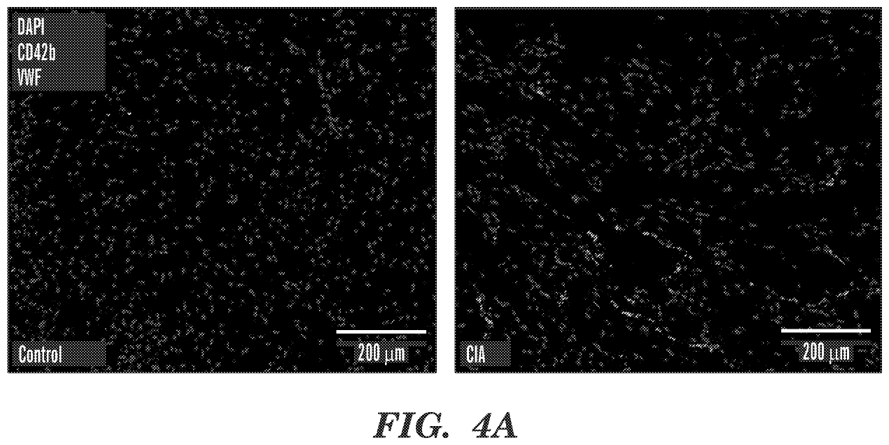

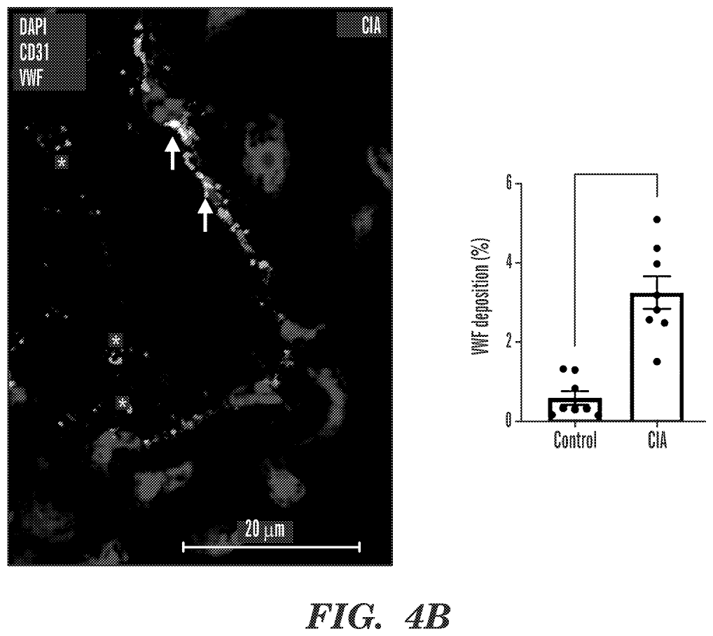

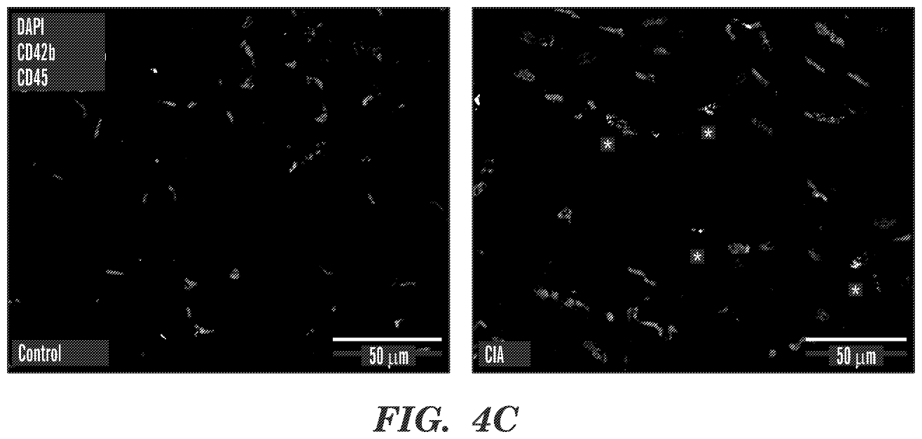

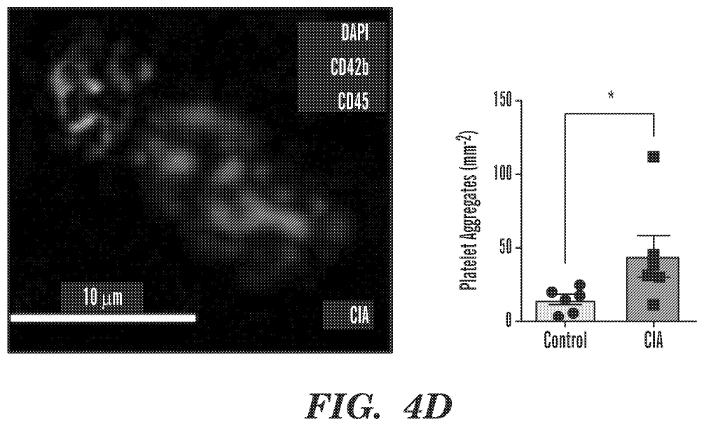

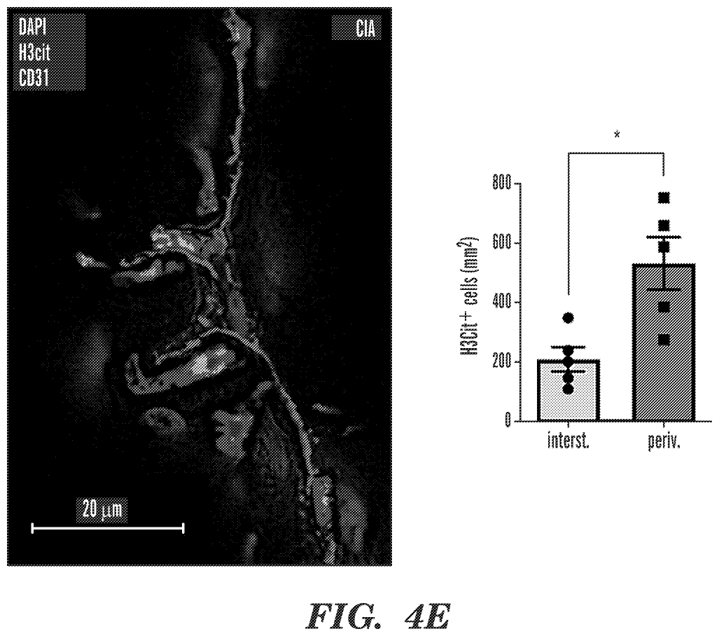

FIG. 4: Thromboinflammation, endothelial activation and activated neutrophils outside the vascular bed are present in RA mice hearts.

FIG. 4A: Representative LV sections and quantification of von Willebrand Factor (VWF) staining, an endothelial adhesive protein deposited in the vasculature. (n=7)

FIG. 4B: Endothelial activation leads to extrusion of endothelial-anchored VWF within the vascular lumen. Arrows indicate VWF near the vessel wall and * indicates intraluminal VWF aggregates; L=Lumen of vessel.

FIG. 4C: Immunofluorescence staining in LV sections of mice with CIA and healthy control mice for accumulating CD42b+ cells indicative of (micro) thrombosis in the vasculature a major feature of thromboinflammation.

FIG. 4D: Macroscopic picture of an aggregate of CD42b⁺ platelets in myocardium of a mouse with CIA and quantification of total number of aggregates per LV section. (n=5)

FIG. 4E: Immunofluorescence staining in LV sections of mice with CIA for H3Cit+ cells in relation to CD31+signal with quantification of H3Cit+ cells. (For each mouse 5 peripheral and 5 perivascular cross section areas were blind chosen and quantified and the resulting total average used for comparative analysis; n=5)

Data are mean±SEM. *P<0.05, **P<0.01, ***P<0.001; Mann-Whitney U-test.

Conclusion: Activation of the myocardial vascular endothelium with extrusion of VWF and hence increased adhesion of activated neutrophils and platelet aggregate formation promotes microthrombosis and neutrophil infiltration in the mice with CIA that is likely detrimental to the heart.

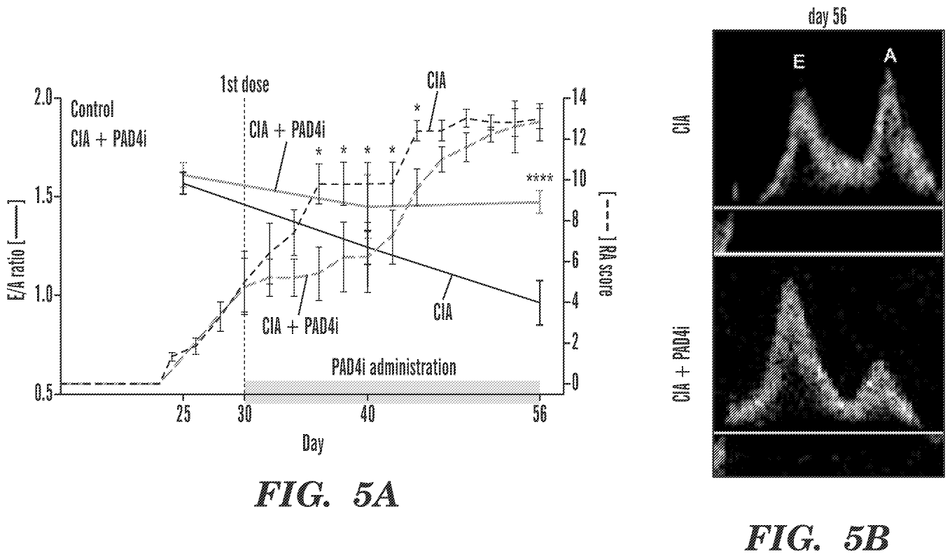

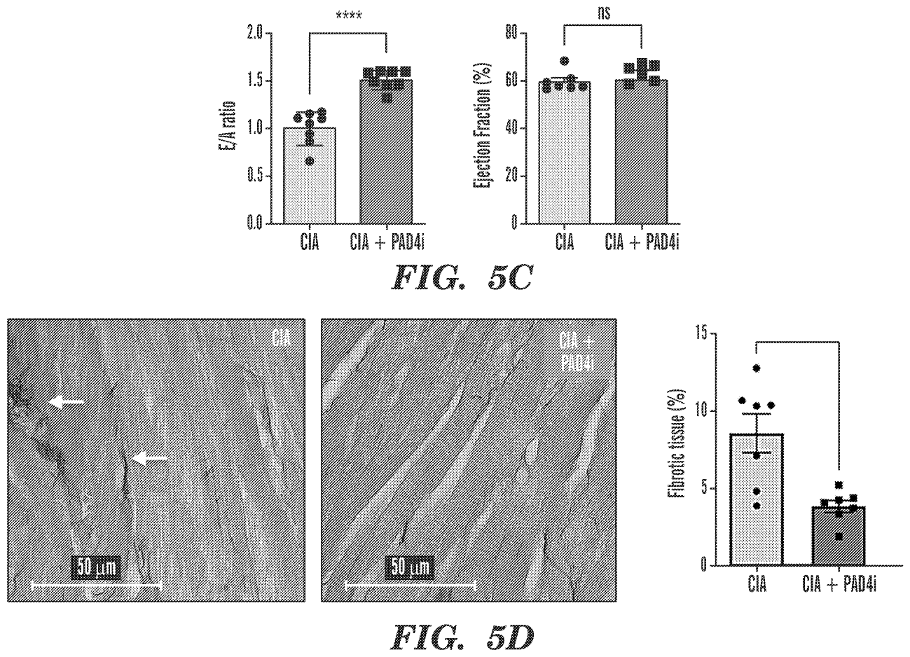

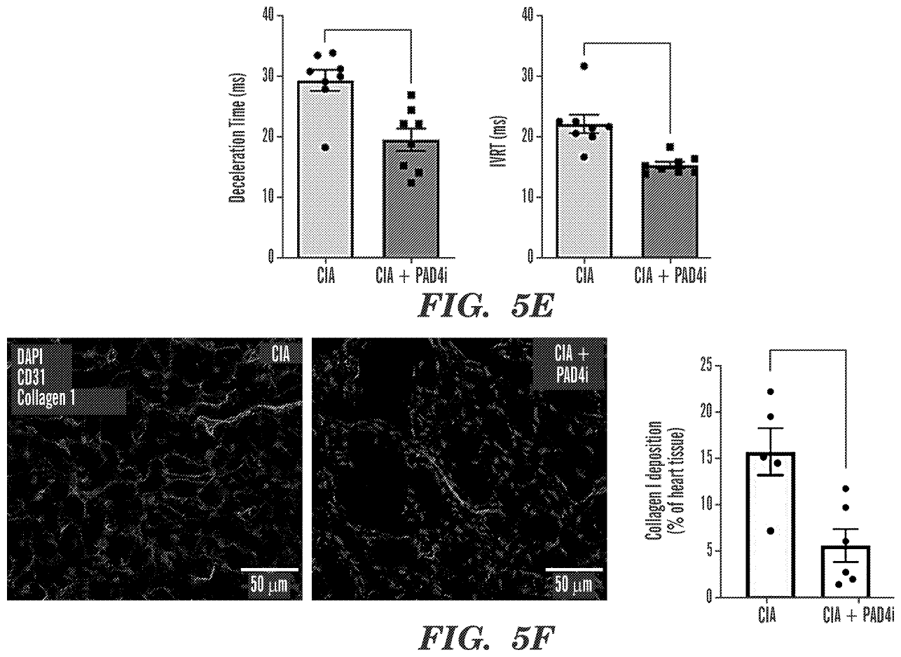

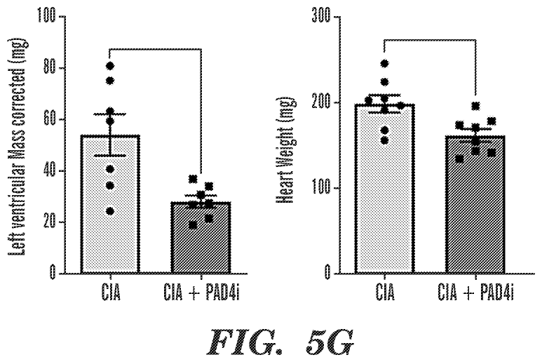

FIG. 5: Treatment with an orally available PAD4 inhibitor preserves LV diastolic function and dampens disease progression in mice with RA.

FIG. 5A: Timecourse illustrating the ratio of early (E) to late (A) diastolic filling velocities across the mitral valve as measured by pulsed wave Doppler echocardiography (E/A ratio; left Y axis; -) in mice with CIA and mice with CIA treated with PAD4 inhibitor. (n=8) Clinical severity of rheumatoid arthritis (RA Score; right y axis; ---) in DBA/1J mice with CIA and mice with CIA treated with PAD4 inhibitor. (n=8)

FIG. 5B: Representative flow patterns, acquired using pulsed-wave Doppler echocardiography, depicting the velocities over the mitral valve at an indicated timepoint in a mouse with CIA and a mouse with CIA treated with PAD4 inhibitor.

FIG. 5C: E/A ratio and ejection fraction (EF) as assessed by echocardiography in mice with CIA and mice with CIA treated with PAD4 inhibitor respectively at day 56. (n=8)

FIG. 5D: Representative LV section images of Sirius Red fast Green Staining Kit and comparative analysis of total fibrotic tissue (red) in CIA and CIA treated with PAD4 inhibitor respectively. Arrows indicate fibrotic tissue. (n=8)

FIG. 5E: Deceleration of time reflecting the duration for equalizing the pressure difference between the left atrium (LA) and the left ventricle (LV) heart weight as assessed by echocardiography (LV mass) in mice with CIA and mice with CIA treated with PAD4 inhibitor. (n=7)

FIG. 5F: Representative Immunofluorescence staining of LV sections from a mouse with CIA and a healthy control for CD31+ cells (green) and Collagen 1 (red). Quantification of total Collagen I deposition per LV section. (n=5/6)

FIG. 5G: LV mass as determined by echocardiography and heart-weight normalized to tibia-length of mice with CIA and mice with CIA treated with PAD4 inhibitor after 56 days. (n=8)

Data are mean±SEM. *P<0.05, **P<0.01, ***P<0.001; Paired Student's t test (D, E, F: Wilcoxon matched pairs signed rank test).

Conclusion: Treatment with PAD4 inhibitor after onset of clinical signs of rheumatoid arthritis delays but does not prevent RA disease progression. However, treatment improved LV function throughout the study. Targeting PAD4 decreases myocardial fibrosis and collagen 1 deposition and thus prevents hypertrophic myocardial remodeling keeping the cardiac cycle intact.

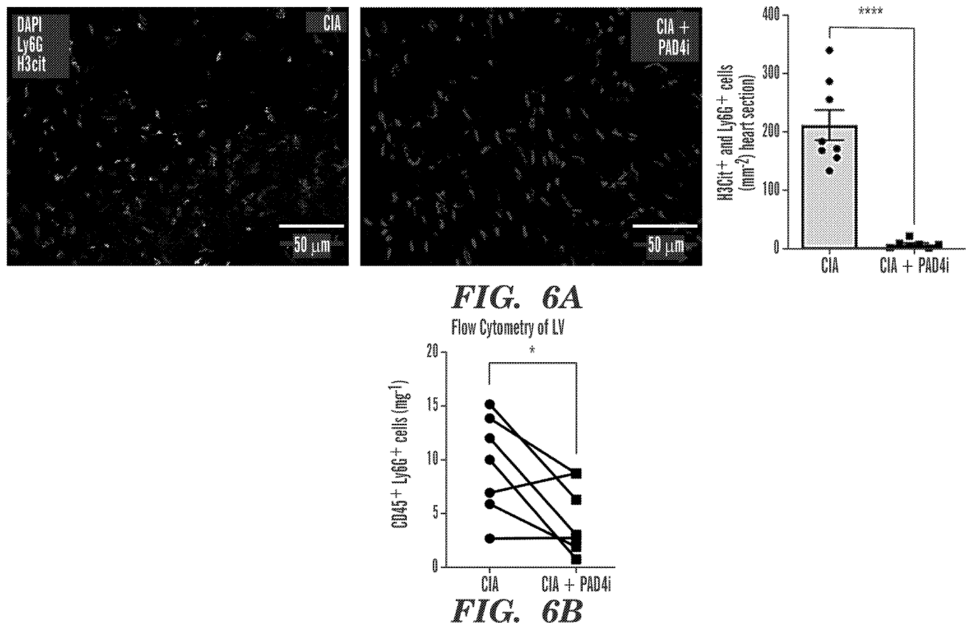

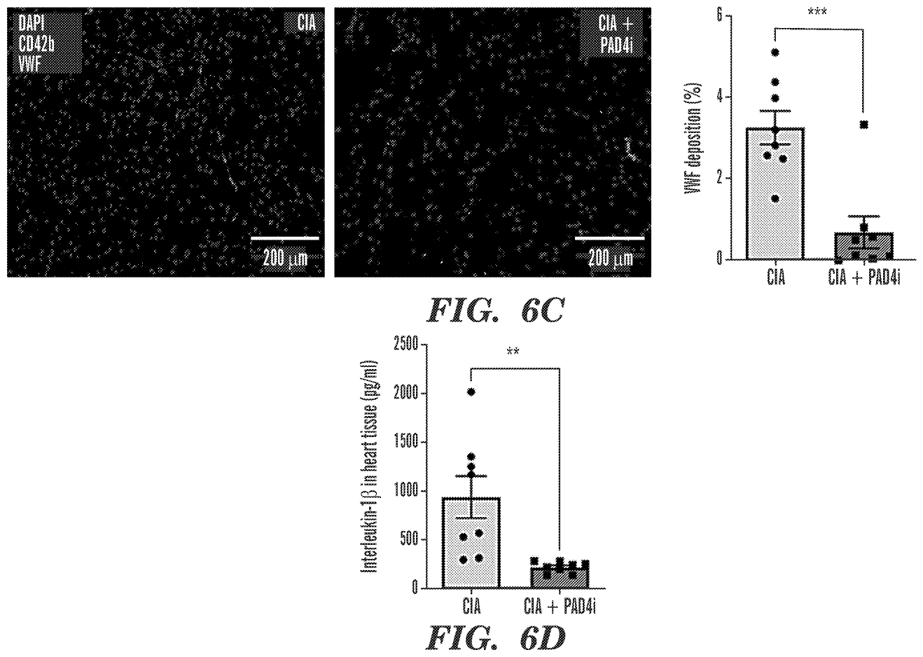

FIG. 6: Treatment of mice with RA using a PAD4 inhibitor decreases neutrophil infiltration as well as endothelial activation in mice with RA after 26 days of treatment.

FIG. 6A: Representative immunostained LV sections and quantification of double positive (Ly6G+ (red) and H3cit+ (green)) cells indicative of activated neutrophils in myocardium of mice with CIA and mice with CIA treated with PAD4 inhibitor. (n=8)

FIG. 6B: Quantification of infiltrating CD45+ and LY6G⁺ cells in myocardial tissue of mice with CIA and mice with CIA treated with PAD4 inhibitor respectively, assessed by flow cytometry (FACS). (n=7)

FIG. 6C: Representative LV sections and Quantification of DAPI+ (blue) and VWF+signal in myocardium of mice with CIA and mice with CIA treated with PAD4 inhibitor.

FIG. 6D: Myocardial tissue levels of Interleukin 1beta an inflammatory cytokine mediating fibrosis and secreted by neutrophils in mice with CIA and mice with CIA treated with PAD4 inhibitor.

Data are mean±SEM. *P<0.05, **P<0.01, ***P<0.001; Paired Student's t test (B: Wilcoxon matched pairs signed rank test).

Conclusion: Treatment with PAD4 inhibitor decreases deposition of activated neutrophils in the myocardium probably through disrupting the thromboinflammatory cycle and decreasing endothelial activation i.e., VWF release.

### REFERENCES

1. Shah S J, Borlaug B A, Kitzman D W, McCulloch A D, Blaxall B C, Agarwal R, et al. Research Priorities for Heart Failure With Preserved Ejection Fraction: National Heart, Lung, and Blood Institute Working Group Summary. Circulation. 2020; 141(12):1001-26.
2. Dunlay S M, Roger V L, Redfield M M. Epidemiology of heart failure with preserved ejection fraction. Nat Rev Cardiol. 2017; 14(10):591-602.
3. Avina-Zubieta J A, Choi H K, Sadatsafavi M, Etminan M, Esdaile J M, Lacaille D. Risk of cardiovascular mortality in patients with rheumatoid arthritis: a meta-analysis of observational studies. Arthritis Rheum. 2008; 59(12):1690-7.
4. Nicola P J, Maradit-Kremers H, Roger V L, Jacobsen S J, Crowson C S, Ballman K V, et al. The risk of congestive heart failure in rheumatoid arthritis: a population-based study over 46 years. Arthritis Rheum. 2005; 52(2):412-20.
5. Myasoedova E, Crowson C S, Nicola P J, Maradit-Kremers H, Davis J M, 3rd, Roger V L, et al. The influence of rheumatoid arthritis disease characteristics on heart failure. J Rheumatol. 2011; 38(8):1601-6.
6. Ahlers M J, Lowery B D, Farber-Eger E, Wang T J, Bradham W, Ormseth M J, et al. Heart Failure Risk Associated With Rheumatoid Arthritis-Related Chronic Inflammation. J Am Heart Assoc. 2020; 9(10):e014661.
7. Maradit-Kremers H, Crowson C S, Nicola P J, Ballman K V, Roger V L, Jacobsen S J, et al. Increased unrecognized coronary heart disease and sudden deaths in rheumatoid arthritis: a population-based cohort study. Arthritis Rheum. 2005; 52(2):402-11.
8. Schau T, Gottwald M, Arbach O, Seifert M, Schopp M, Neuss M, et al. Increased Prevalence of Diastolic Heart Failure in Patients with Rheumatoid Arthritis Correlates with Active Disease, but Not with Treatment Type. J Rheumatol. 2015; 42(11):2029-37.
9. Mantel A, Holmqvist M, Andersson D C, Lund L H, Askling J. Association Between Rheumatoid Arthritis and Risk of Ischemic and Nonischemic Heart Failure. J Am Coll Cardiol. 2017; 69(10):1275-85.
10. Paulus W J, Zile M R. From Systemic Inflammation to Myocardial Fibrosis: The Heart Failure With Preserved Ejection Fraction Paradigm Revisited. Circ Res. 2021; 128(10):1451-67.
11. Chen J, Norling L V, Cooper D. Cardiac Dysfunction in Rheumatoid Arthritis: The Role of Inflammation. Cells. 2021; 10(4).
12. Ridker P M, Everett B M, Pradhan A, MacFadyen J G, Solomon D H, Zaharris E, et al. Low-Dose Methotrexate for the Prevention of Atherosclerotic Events. N Engl J Med. 2019; 380(8):752-62.
13. Roubille C, Richer V, Starnino T, McCourt C, McFarlane A, Fleming P, et al. The effects of tumour necrosis factor inhibitors, methotrexate, non-steroidal anti-inflammatory drugs and corticosteroids on cardiovascular events in rheumatoid arthritis, psoriasis and psoriatic arthritis: a systematic review and meta-analysis. Ann Rheum Dis. 2015; 74(3):480-9.
14. Chung E S, Packer M, Lo K H, Fasanmade A A, Willerson J T, Anti TNFTACHFI. Randomized, double-blind, placebo-controlled, pilot trial of infliximab, a chimeric monoclonal antibody to tumor necrosis factor-alpha, in patients with moderate-to-severe heart failure: results of the anti-TNF Therapy Against Congestive Heart Failure (ATTACH) trial. Circulation. 2003; 107(25):3133-40.
15. Pfeffer M A, Shah A M, Borlaug B A. Heart Failure With Preserved Ejection Fraction In Perspective. Circ Res. 2019; 124(11):1598-617.
16. Bradshaw A D, Baicu C F, Rentz T J, Van Laer A O, Boggs J, Lacy J M, et al. Pressure overload-induced alterations in fibrillar collagen content and myocardial diastolic function: role of secreted protein acidic and rich in cysteine (SPARC) in post-synthetic procollagen processing. Circulation. 2009; 119(2):269-80.
17. Garg P, Assadi H, Jones R, Chan W B, Metherall P, Thomas R, et al. Left ventricular fibrosis and hypertrophy are associated with mortality in heart failure with preserved ejection fraction. Sci Rep. 2021; 11(1):617.
18. Kanagala P, Cheng ASH, Singh A, Khan J N, Gulsin G S, Patel P, et al. Relationship Between Focal and Diffuse Fibrosis Assessed by CMR and Clinical Outcomes in Heart Failure With Preserved Ejection Fraction. JACC Cardiovasc Imaging. 2019; 12(11 Pt 2):2291-301.
19. Duprez D A, Gross M D, Kizer J R, Ix J H, Hundley W G, Jacobs D R, Jr. Predictive Value of Collagen Biomarkers for Heart Failure With and Without Preserved Ejection Fraction: MESA (Multi-Ethnic Study of Atherosclerosis). J Am Heart Assoc. 2018; 7(5).
20. De Marco C, Claggett B L, de Denus S, Zile M R, Huynh T, Desai A S, et al. Impact of diabetes on serum biomarkers in heart failure with preserved ejection fraction: insights from the TOPCAT trial. ESC Heart Fail. 2021; 8(2):1130-8.
21. Cunningham J W, Claggett B L, O'Meara E, Prescott M F, Pfeffer M A, Shah S J, et al. Effect of Sacubitril/Valsartan on Biomarkers of Extracellular Matrix Regulation in Patients With HFpEF. J Am Coll Cardiol. 2020; 76(5):503-14.
22. Albar Z, Albakri M, Hajjari J, Karnib M, Janus S E, Al-Kindi S G. Inflammatory Markers and Risk of Heart Failure With Reduced to Preserved Ejection Fraction. Am J Cardiol. 2022; 167:68-75.
23. Sanders-van Wijk S, Tromp J, Beussink-Nelson L, Hage C, Svedlund S, Saraste A, et al. Proteomic Evaluation of the Comorbidity-Inflammation Paradigm in Heart Failure With Preserved Ejection Fraction: Results From the PROMIS-HFpEF Study. Circulation. 2020; 142(21):2029-44.
24. Sanders-van Wijk S, van Empel V, Davarzani N, Maeder M T, Handschin R, Pfisterer M E, et al. Circulating biomarkers of distinct pathophysiological pathways in heart failure with preserved vs. reduced left ventricular ejection fraction. Eur J Heart Fail. 2015; 17(10):1006-14.
25. Chow S L, Maisel A S, Anand I, Bozkurt B, de Boer R A, Felker G M, et al. Role of Biomarkers for the Prevention, Assessment, and Management of Heart Failure: A Scientific Statement From the American Heart Association. Circulation. 2017; 135(22):e1054-e91.
26. Klopf J, Brostjan C, Eilenberg W, Neumayer C. Neutrophil Extracellular Traps and Their Implications in Cardiovascular and Inflammatory Disease. Int J Mol Sci. 2021; 22(2).
27. Wright H L, Moots R J, Edwards S W. The multifactorial role of neutrophils in rheumatoid arthritis. Nat Rev Rheumatol. 2014; 10(10):593-601.
28. Voors A A, Anker S D, Cleland J G, Dickstein K, Filippatos G, van der Harst P, et al. A systems BIOlogy Study to TAilored Treatment in Chronic Heart Failure: rationale, design, and baseline characteristics of BIOSTAT-CHF. Eur J Heart Fail. 2016; 18(6):71626.
29. Sorvillo N, Cherpokova D, Martinod K, Wagner D D. Extracellular DNA NET-Works With Dire Consequences for Health. Circ Res. 2019; 125(4):470-88.
30. Herrero-Cervera A, Soehnlein O, Kenne E. Neutrophils in chronic inflammatory diseases. Cell Mol Immunol. 2022; 19(2):177-91.
31. Thiam H R, Wong S L, Qiu R, Kittisopikul M, Vahabikashi A, Goldman A E, et al. NETosis proceeds by cytoskeleton and endomembrane disassembly and PAD4-mediated chromatin decondensation and nuclear envelope rupture. Proc Natl Acad Sci USA. 2020; 117(13):7326-37.
32. Brinkmann V, Zychlinsky A. Beneficial suicide: why neutrophils die to make NETs. Nat Rev Microbiol. 2007; 5(8):577-82.
33. Papadaki G, Kambas K, Choulaki C, Vlachou K, Drakos E, Bertsias G, et al. Neutrophil extracellular traps exacerbate Th1-mediated autoimmune responses in rheumatoid arthritis by promoting D C maturation. Eur J Immunol. 2016; 46(11):254254.
34. Chrysanthopoulou A, Gkaliagkousi E, Lazaridis A, Arelaki S, Pateinakis P, Ntinopoulou M, et al. Angiotensin II triggers release of neutrophil extracellular traps, linking thromboinflammation with essential hypertension. JCI Insight. 2021; 6(18).
35. Rabinovitch M. Molecular pathogenesis of pulmonary arterial hypertension. J Clin Invest. 2012; 122(12):4306-13.
36. Schermuly R T, Ghofrani H A, Wilkins M R, Grimminger F. Mechanisms of disease: pulmonary arterial hypertension. Nat Rev Cardiol. 2011; 8(8):443-55.
37. Aldabbous L, Abdul-Salam V, McKinnon T, Duluc L, Pepke-Zaba J, Southwood M, et al. Neutrophil Extracellular Traps Promote Angiogenesis: Evidence From Vascular Pathology in Pulmonary Hypertension. Arterioscler Thromb Vasc Biol. 2016; 36(10):2078-87.
38. Cahilog Z, Zhao H, Wu L, Alam A, Eguchi S, Weng H, et al. The Role of Neutrophil NETosis in Organ Injury: Novel Inflammatory Cell Death Mechanisms. Inflammation. 2020; 43(6):2021-32.
39. Saffarzadeh M, Juenemann C, Queisser M A, Lochnit G, Barreto G, Galuska S P, et al. Neutrophil extracellular traps directly induce epithelial and endothelial cell death: a predominant role of histones. PLoS One. 2012; 7(2):e32366.
40. Pietronigro E C, Della Bianca V, Zenaro E, Constantin G. NETosis in Alzheimer's Disease. Front Immunol. 2017; 8:211.
41. Martinod K, Witsch T, Erpenbeck L, Savchenko A, Hayashi H, Cherpokova D, et al. Peptidylarginine deiminase 4 promotes age-related organ fibrosis. J Exp Med. 2017; 214(2):439-58.
42. Desai O, Winkler J, Minasyan M, Herzog E L. The Role of Immune and Inflammatory Cells in Idiopathic Pulmonary Fibrosis. Frontiers in medicine. 2018; 5:43.
43. Huang E, Peng N, Xiao F, Hu D, Wang X, Lu L. The Roles of Immune Cells in the Pathogenesis of Fibrosis. Int J Mol Sci. 2020; 21(15).
44. Kobayashi H, Kobayashi Y, Yokoe I, Akashi Y, Takei M, Giles J T. Magnetic Resonance Imaging-Detected Myocardial Inflammation and Fibrosis in Rheumatoid Arthritis: Associations With Disease Characteristics and N-Terminal Pro-Brain Natriuretic Peptide Levels. Arthritis Care Res (Hoboken). 2017; 69(9):1304-11.
45. Tang X, Wang P, Zhang R, Watanabe I, Chang E, Vinayachandran V, et al. KLF2 regulates neutrophil activation and thrombosis in cardiac hypertrophy and heart failure progression. J Clin Invest. 2022; 132(3).
46. Castanheira FVS, Kubes P. Neutrophils and NETs in modulating acute and chronic inflammation. Blood. 2019; 133(20):2178-85.
47. Papayannopoulos V. Neutrophil extracellular traps in immunity and disease. Nat Rev Immunol. 2018; 18(2):134-47.
48. Zhang X L, Wang T Y, Chen Z, Wang H W, Yin Y, Wang L, et al. HMGB1-Promoted Neutrophil Extracellular Traps Contribute to Cardiac Diastolic Dysfunction in Mice. J Am Heart Assoc. 2022; 11(4):e023800.
49. Chen J, Norling L V, Mesa J G, Silva M P, Burton S E, Reutelingsperger C, et al. Annexin A1 attenuates cardiac diastolic dysfunction in mice with inflammatory arthritis. Proc Natl Acad Sci USA. 2021; 118(38).
50. Fukui S, Gutch S, Fukui S, Cherpokova D, Aymonnier K, Sheehy C E, et al. The Prominent Role of Hematopoietic Peptidyl Arginine Deiminase 4 in Arthritis: Collagen- and Granulocyte Colony-Stimulating Factor-Induced Arthritis Model in C57BL/6 Mice. Arthritis Rheumatol. 2022; 74(7):1139-46.
51. Martinod K, Demers M, Fuchs T A, Wong S L, Brill A, Gallant M, et al.

Neutrophil histone modification by peptidylarginine deiminase 4 is critical for deep vein thrombosis in mice. Proc Natl Acad Sci USA. 2013; 110(21):8674-9.
52. Sreejit G, Nooti S K, Jaggers R M, Athmanathan B, Ho Park K, Al-Sharea A, et al. Retention of the NLRP3 Inflammasome-Primed Neutrophils in the Bone Marrow Is Essential for Myocardial Infarction-Induced Granulopoiesis. Circulation. 2022; 145(1):31-44.
53. Vats R, Kaminski T W, Brzoska T, Leech J A, Tutuncuoglu E, Katoch O, et al. Liver-to-lung microembolic NETs promote gasdermin D-dependent inflammatory lung injury in sickle cell disease. Blood. 2022; 140(9):1020-37.
54. Wong S L, Demers M, Martinod K, Gallant M, Wang Y, Goldfine A B, et al. Diabetes primes neutrophils to undergo NETosis, which impairs wound healing. Nat Med. 2015; 21(7):815-9.
55. Wang Y, Sano S, Oshima K, Sano M, Watanabe Y, Katanasaka Y, et al. Wnt5a-Mediated Neutrophil Recruitment Has an Obligatory Role in Pressure Overload-Induced Cardiac Dysfunction. Circulation. 2019; 140(6):487-99.
56. He L, Liu R, Yue H, Zhu G, Fu L, Chen H, et al. NETs promote pathogenic cardiac fibrosis and participate in ventricular aneurysm formation after ischemia injury through the facilitation of perivascular fibrosis. Biochem Biophys Res Commun. 2021; 583:15461.
57. Wilson A S, Randall K L, Pettitt J A, Ellyard J I, Blumenthal A, Enders A, et al. Neutrophil extracellular traps and their histones promote Th17 cell differentiation directly via TLR2. Nat Commun. 2022; 13(1):528.
58. Lee K Y, Ho S C, Lin H C, Lin S M, Liu C Y, Huang C D, et al. Neutrophil-derived elastase induces TGF-beta1 secretion in human airway smooth muscle via N F-kappaB pathway. Am J Respir Cell Mol Biol. 2006; 35(4):407-14.
59. Pan X, Chen Z, Huang R, Yao Y, Ma G. Transforming growth factor beta1 induces the expression of collagen type I by DNA methylation in cardiac fibroblasts. PLoS One. 2013; 8(4):e60335.
60. Frangogiannis N. Transforming growth factor-beta in tissue fibrosis. J Exp Med. 2020; 217(3):e20190103.
61. Chrysanthopoulou A, Mitroulis I, Apostolidou E, Arelaki S, Mikroulis D, Konstantinidis T, et al. Neutrophil extracellular traps promote differentiation and function of fibroblasts. J Pathol. 2014; 233(3):294-307.
62. Collier D M, Villalba N, Sackheim A, Bonev A D, Miller Z D, Moore J S, et al. Extracellular histones induce calcium signals in the endothelium of resistance-sized mesenteric arteries and cause loss of endothelium-dependent dilation. Am J Physiol Heart Circ Physiol. 2019; 316(6):H1309-H22.
63. Gibb A A, Lazaropoulos M P, Elrod J W. Myofibroblasts and Fibrosis. Circulation Research. 2020; 127(3):427-47.
64. Saltiel A R, Olefsky J M. Inflammatory mechanisms linking obesity and metabolic disease. J Clin Invest. 2017; 127(1):1-4.
65. Packer M. Epicardial Adipose Tissue May Mediate Deleterious Effects of Obesity and Inflammation on the Myocardium. J Am Coll Cardiol. 2018; 71(20):2360-72.
66. Mouton A J, Li X, Hall M E, Hall J E. Obesity, Hypertension, and Cardiac Dysfunction: Novel Roles of Immunometabolism in Macrophage Activation and Inflammation. Circ Res. 2020; 126(6):789-806.
67. Samson R, Le Jemtel T H. Therapeutic Stalemate in Heart Failure With Preserved Ejection Fraction. J Am Heart Assoc. 2021; 10(12):e021120.
68. Nauta J F, Hummel Y M, Tromp J, Ouwerkerk W, van der Meer P, Jin X, et al. Concentric vs. eccentric remodelling in heart failure with reduced ejection fraction: clinical characteristics, pathophysiology and response to treatment. Eur J Heart Fail. 2020; 22(7):1147-55.
69. Hanna A, Frangogiannis N G. Inflammatory Cytokines and Chemokines as Therapeutic Targets in Heart Failure. Cardiovasc Drugs Ther. 2020; 34(6):849-63.
70. Melendez G C, McLarty J L, Levick S P, Du Y, Janicki J S, Brower G L. Interleukin 6 mediates myocardial fibrosis, concentric hypertrophy, and diastolic dysfunction in rats. Hypertension. 2010; 56(2):225-31.
71. Norton G R, Peterson V R, Robinson C, Norman G, Libhaber C D, Libhaber E, et al. Independent of left ventricular mass, circulating inflammatory markers rather than pressure load are associated with concentric left ventricular remodelling. Int J Cardiol. 2019; 274:342-7.
72. Yokoe I, Kobayashi H, Kobayashi Y, Giles J T, Yoneyama K, Kitamura N, et al. Impact of tocilizumab on N-terminal pro-brain natriuretic peptide levels in patients with active rheumatoid arthritis without cardiac symptoms. Scand J Rheumatol. 2018; 47(5):364-70.
73. Obokata M, Reddy Y N V, Melenovsky V, Pislaru S, Borlaug B A. Deterioration in right ventricular structure and function over time in patients with heart failure and preserved ejection fraction. Eur Heart J. 2019; 40(8):689-97.
74. Wang Z, Zhao Y T, Zhao T C. Histone deacetylases in modulating cardiac disease and their clinical translational and therapeutic implications. Exp Biol Med (Maywood). 2021; 246(2):213-25.
75. Chaar D, Dumont B L, Vulesevic B, Neagoe P E, Rakel A, White M, et al. Neutrophils and Circulating Inflammatory Biomarkers in Diabetes Mellitus and Heart Failure With Preserved Ejection Fraction. Am J Cardiol. 2022; 178:80-8.
76. Zhang L, Yuan Y, Xu Q, Jiang Z, Chu C Q. Contribution of neutrophils in the pathogenesis of rheumatoid arthritis. J Biomed Res. 2019; 34(2):86-93.
77. Komajda M, Carson P E, Hetzel S, McKelvie R, McMurray J, Ptaszynska A, et al. Factors associated with outcome in heart failure with preserved ejection fraction: findings from the Irbesartan in Heart Failure with Preserved Ejection Fraction Study (I-PRESERVE). Circ Heart Fail. 2011; 4(1):27-35.
78. Wagner D D, Heger L A. Thromboinflammation: From Atherosclerosis to COVID-19. Arterioscler Thromb Vasc Biol. 2022; 42(9):1103-12.
79. Gragnano F, Sperlongano S, Golia E, Natale F, Bianchi R, Crisci M, et al. The Role of von Willebrand Factor in Vascular Inflammation: From Pathogenesis to Targeted Therapy. Mediators Inflamm. 2017; 2017:5620314.
80. Wong S L, Wagner D D. Peptidylarginine deiminase 4: a nuclear button triggering neutrophil extracellular traps in inflammatory diseases and aging. FASEB J. 2018:fj201800691R.
81. Hejblum B P, Cui J, Lahey L J, Cagan A, Sparks J A, Sokolove J, et al. Association Between Anti-Citrullinated Fibrinogen Antibodies and Coronary Artery Disease in Rheumatoid Arthritis. Arthritis Care Res (Hoboken). 2018; 70(7):1113-7.
82. Borissoff J I, Joosen I A, Versteylen M O, Brill A, Fuchs T A, Savchenko A S, et al. Elevated levels of circulating DNA and chromatin are independently associated with severe coronary atherosclerosis and a prothrombotic state. Arterioscler Thromb Vasc Biol. 2013; 33(8):2032-40.
83. Mao L, Mostafa R, Ibili E, Fert-Bober J. Role of protein deimination in cardiovascular diseases: potential new avenues for diagnostic and prognostic biomarkers. Expert Rev Proteomics. 2021; 18(12):1059-71.
84. Helseth R, Shetelig C, Andersen G O, Langseth M S, Limalanathan S, Opstad T B, et al. Neutrophil Extracellular Trap Components Associate with Infarct Size, Ventricular Function, and Clinical Outcome in STEMI. Mediators Inflamm. 2019; 2019:7816491.

### Example 2

### Introduction

The activity of protein arginine deiminase 4 (PAD4) is comprehensively involved in the pathophysiology of rheumatoid arthritis (RA) partly through the production of immunogenic neoepitopes. (Sebbag, Chapuy-Regaud et al. 2004) Accordingly, altered PAD4 activity is amongst the early indicators preceding the onset of RA. (Koushik, Joshi et al. 2017) It has been shown that prophylactic treatment with the non-covalent PAD4 inhibitor JBI-589 alleviates arthritis in a mouse model. (Gajendran, Fukui et al. 2023) The effect of therapeutic PAD4 inhibition on disease severity and the extent of the incidental collateral damage after onset of arthritis, however, is not defined. In neutrophils, activation and nuclear translocation of PAD4 facilitates histone modification, an essential step in the process of the formation of neutrophil extracellular traps (NETs). (Martinod, Demers et al. 2013, Wong und Wagner 2018, Thiam, Wong et al. 2020) Accumulating evidence points to NETs as an important risk factor for thrombosis with associated inflammation (thromboinflammation) in arthritis. (Song, Ye et al. 2020, Fukui, Gutch et al. 2022) The clinical relevance of aberrant thromboinflammation in arthritis is supported by an increased risk of adverse myocardial remodeling and heart failure (HF) among affected patients. (Ahlers, Lowery et al. 2020) Current anti-inflammatory RA therapies, although effective in improving joint disease severity, appear to have little to no cardioprotective effect, highlighting the need for the development of better targeted therapies. (Roubille, Richer et al. 2015, Baoqi, Dan et al. 2021) It has been shown, using a murine acute Myocardial Infarction/reperfusion injury model, that targeting early leukocyte recruitment and formation of NETs offers significant protection against myocardial damage. (Savchenko, Borissoff et al. 2014) Indeed, evidence indicates, that immune cell infiltration in the myocardium can have adverse effects on the heart and contribute to the pathogenesis of HF in a chronic setting as well. (Carrillo-Salinas, Ngwenyama et al. 2019) It has been hypothesized that targeting PAD4 could break a vicious cycle of NETs triggering the release of von Willebrand factor (VWF) and P-selectin from Weibel-Palade bodies which in turn facilitate increased leukocyte recruitment. Together, with the ability of extracellular PAD4 to reduce VWF-platelet string clearance and accelerate formation of stable platelet plugs, this provides the basis for aberrant thromboinflammation. (Sorvillo, Mizurini et al. 2019)

In addition to its enzymatic role in chromatin decondensation and NET release, PAD4 is also critically involved in neutrophil production of Interleukin-1p (Il-1p), a biomarker of thromboinflammation, via regulation of NLR family pyrin domain containing 3 (NLRP3) inflammasome activity. (Munzer, Negro et al. 2021) Correspondingly, in a model of murine peritonitis, the absence of NLRP3 inflammasome activity also affects neutrophil recruitment. (Fukui, Fukui et al. 2022)

It was shown that deposition of von Willebrand Factor (VWF) in the synovium and subsequent pathogenic NET retention promotes arthritis and established a critical role of PAD4. (Fukui, Gutch et al. 2022, Fukui, Gutch et al. 2022)

It was speculated, that elevated PAD4 activity, as described in the well-established model of Collagen Induced Arthritis (CIA) in the DBA/1 strain, could drive systemic thromboinflammation as well as neutrophil activation and recruitment to the myocardium thus mediating adverse myocardial remodeling in the late stages of the disease. The effort to investigate the role of PAD4 was facilitated by the recent description of an orally available selective PAD4-inhibitor, JBI-589. (Gajendran, Fukui et al. 2023)

### Methods

### Animals

DBA/1 J mice were purchased from Jackson Laboratory. All mice were kept specific pathogen free. Experimental protocols were approved by the Institutional Animal Care and Use Committee of Boston Children's Hospital (Protocol number: 20-01-4096R). To conduct a time-course study investigating onset and persistence of the cardiac dysfunction in DBA/1 J, the following groups were studied: DBA/1 J with CIA, DBA/1 J age-matched wild type, DBA/1 J with CIA treated with PAD4 inhibitor, DBA/1 J with CIA treated with 0.5% Methylcellulose (vehicle of the PAD4 inhibitor). The PAD4 specific inhibitor JBI-589 was provided by Jubilant Therapeutics (JBI-589, (R)-(3-aminopiperidin-1-yl)(2-(1-(4-fluorobenzyl)-1H-indol-2-yl)-3-methylimidazo[1,2-a]pyridin-7-yl)methanone). Mice received JBI-589 at a dose of 10 mg/kg via oral gavage for 26 consecutive days. JBI-589 was given in suspension formulation prepared using Tween-80 and 0.5% methyl cellulose. For the PAD4 inhibitor trial, mice were paired depending on their RA severity score and randomly allocated to treatment or control group. All recurring events including animal scoring and administration of inhibitor was done at similar times during the day. All groups were age and sex matched and were fed ad libitum with free access to water. All procedures conform with the NIH Guide for the Care and Use of Laboratory Animals. Experimental protocols were approved by the Institutional Animal Care and Use Committee of Boston Children's Hospital (Protocol number: 20-01-4096R).

### Collagen Induced Arthritis Model & Scoring

DBA/1 J mice aged 8-9 weeks were immunized with an emulsion of 50 μl CFA (Chondrex, Inc.; Catalog no. 7023) and 50 μl of bovine type II collagen (Chondrex, Inc.; catalog no. 20012) injected intra-dermally into the base of the tail on day 0. On day 21, a booster immunization of type II collagen with Freund's incomplete adjuvant (IFA) (catalog no. 7002; Chondrex) was administered intra-dermally at a site proximal to the first injection site. Mice were assessed for development of arthritis using the semi-quantitative, mouse arthritis scoring system provided by Chondrex (chondrex.com). This protocol is based on hind-foot examination with range of 0 (no inflammation) to 4 (erythema and severe swelling encompassing ankle, foot, and digits). Evaluation of arthritis severity was performed blinded by two independent evaluators.

### Echocardiography

B-Mode, M-mode, and Doppler echocardiography were performed in DBA/1 J mice with CIA, DBA/1 J mice with CIA treated with PAD4 inhibitor or saline administration and age-matched DBA/1 J mice at day 0, day 25, day 45 and day 56 for time-course analysis of cardiac function. Anesthesia was induced with 3% isoflurane and maintained at 1.5 to 2% for the duration of the procedure. Warmed echo gel was placed on the shaved chest. Body temperature was regulated through a heat pad and heart rate measured with an electrocardiogram, both were kept consistent between experimental groups (37° Celcius and 400-500 bpm). Echocardiography images were recorded using a Vevo-3100 imaging system with a 25-55-MHz linear probe (MX550D; VisualSonics, Toronto, Canada). Percentage of ejection fraction was calculated in parasternal long-axis view. M-mode was measured at the papillary muscle level in the short-axis view. Parameters measured in M-Mode/SAX include: Fractional shortening (FS), Left Ventricular Posterior Wall thickness (LV PWd) in diastole, Left Ventricular Internal Diameter in diastole (LVIDd), From these measured values, other estimates were calculated by computerized algorithm of the echocardiograph (Vevo LAB ultrasound analysis software version 5.7.1; FUJIFILM VisualSonics) was used for calculations, such as Left Ventricular Mass (LV Mass=0.8×(1.04×(((LVEDD+IVSd+PWd)3-LVEDD3)))+0.6) or relative wall thickness (RWT; calculated as 2 times the LVPWd divided by the left ventricular diastolic diameter). Diastolic transmitral left ventricle (LV) inflow images were acquired from apical four-chamber views using pulsed-wave Doppler to calculate early (E) and late (atrial, A) peak filling blood flow velocities, isovolumetric relaxation time (IVRT), and E-wave deceleration time (DT). Tissue Doppler imaging (TDI) to measure tissue motion velocity from the mitral annulus was used to confirm Doppler measurements. The E/A ratio represents the ratio of E wave to A wave. The measurement probe was positioned at the tips of mitral valve leaflet in the mitral valve annulus with the ultrasound beam positioned parallel to the direction of blood flow. All measurements were obtained in triplicate and averaged.

### Quantification of Collagen Deposition and Cardiomyocyte Size

Mice were sacrificed by cervical dislocation under deep anesthesia with isoflurane (3-4% in oxygen) after verification of sufficient analgesia by a lack of visible response to a footpad squeeze. Euthanasia was confirmed with monitoring the mouse for respirations for at least one minute after cervical dislocation. Hearts were collected and residual blood cleared by retrograde perfusion with 5 ml ice cold PBS buffer. Hearts were fixed in 4% paraformaldehyde solution for 24 hours specimens and afterwards dehydrated in alcohol, embedded in paraffin, and cut into 8-m thick serial slides. After deparaffinization through Xyline and rehydration through different grades of alcohols to distilled water, sections were fixed in Bouin's Solution (Sigma-Aldrich) over night. After that, sections were stained according to the Masson's trichrome Staining Kit (Sigma-Aldrich) manufacturers guidelines. In addition, a collagen specific Sirius Red/Fast Green Collagen Staining Kit (Chondrex Inc; catalog #9046) was performed according to manufacturer's protocols. For quantification, photographs of heart sections were taken by bright-field microscopy and quantified by an investigator blinded to the identity of the samples using ImageJ. For quantification of perivascular fibrosis 5 pictures of the interstitial area and perivascular area were chosen at random for quantification and the average used for comparative analysis.

For cryosectioning, tissue samples were snap frozen in O.C.T (Tissue-Tek; product Code 4583). Wheat germ agglutinin (WGA; Invitrogen™; Catalog number: W 11261), labeling glycoproteins of the cell membrane was used for cardiomyocyte cell size quantification. Heart sections were fixed in 4% paraformaldehyde solution for 15 minutes at 37° C. WGA conjugate (concentration 5 g/ml) was applied followed by incubation for 30 minutes at 37° C. After washing, sections were permeabilized with 0.1% Triton X and counterstained with Hoechst 33342 (1:10000; Invitrogen). Cardiomyocyte Area (μm2) was calculated using ImageJ cross-sectional analyzer (https://imagej.net/plugins/cross-sectional-analyzer) of an average of at least 400 individual cells. Both Collagen inoculation and tissue harvest were done in the morning to allow for comparable physiological environments.

### Immunostaining

Cryosections of 8-μm were fixed in 4% paraformaldehyde, permeabilized with 0.1% Triton, blocked in 3% BSA, and incubated overnight at 4° C. with the following primary antibodies: Anti-Collagen I (Abcam; ab21286); anti-Ly6G (clone 1A8; BioLegend); anti-H3Cit (Abcam; ab5103); anti-CD31 (BD Pharmingen; 553370); anti-VWF (DakoCytomation; P0226); anti-CD42b (Emfret; M040-0); anti-CD45 (Abcam; ab154885); anti-TGF beta (Abcam; ab66043); anti-alpha smooth muscle actin (Abcam; ab5694), and anti-CD68 (Abcam; ab53444). After washing, sections were stained with the respective Alexa Fluor-conjugated secondary antibodies (Alexa Fluor 488 donkey anti-rabbit [Invitrogen; Cat. Nr. A21206]IgG and Alexa Fluor 555 goat anti-rat [Invitrogen; Cat. Nr. A21434]IgG; Alexa Fluor 647 goat anti-rabbit [Invitrogen; Cat. Nr. A21244]) and counterstained with Hoechst 33342 (1:10000; Invitrogen; Catalog number: H3570).

### Neutrophil Isolation & NETosis Assay

Peripheral neutrophils were isolated as previously described (Martinod, Demers et al. 2013). Briefly, blood was drawn into 15 mM EDTA, 1% ultra-low endotoxin BSA (1:2 vol/vol). After removal of plasma, cells were resuspended in PBS and layered onto a discontinuous Percoll gradient (78%/69%/52% made isotonic with the addition of 10×PBS, Merck). Samples were centrifuged for 32 min at 1500 g at room temperature, without breaks. After several washing steps, isolated neutrophils were resuspended in RPMI+10 mM HEPES (Gibco™; Catalog number: 15630080) and plated in a 96 wells plate in a concentration of 10-15×10³ neutrophils per well. Neutrophils were allowed to adhere to the glass bottom for 15 minutes at 37° C. and 5% C02 after which they were and fixed with 4% paraformaldehyde followed again by permeabilization, blocking and immunostaining with anti-H3Cit (Abcam) and counterstaining with Hoechst 33342 (1:10000; Invitrogen; Catalog number: H3570).

### Flow Cytometry

After sacrifice, the heart was immediately perfused with 5 mL ice-cold 0.05% EDTA in PBS. The perfusion needle was inserted into the LV and the right atrium cut with a scissor. Paleness of the coronary arteries and cardiac veins was visually verified before cardiac excision. Apical sections, representative of LV tissue, were removed, then placed into ice-cold PBS and afterwards minced. Minced sections were incubated in a solution containing 1.5 mg/mL collagenase (Merck, Product Nr. C8051) for 45 min at 37° C. After filtration through a 70 m cell strainer, the single-cell suspension was centrifuged and the pellet resuspended in Dulbecco's Modified Eagle Medium (DMEM, Thermo Fisher Scientific—US; Catalog Number 11965092) topped up with cell debris remover (Milteny Biotec; Order Number 130-109-398) and PBS followed by another centrifugation at 3000 g. This separated the mononuclear cells from cellular debris. After centrifugation, both the upper- and interphase were discarded. After resuspension with FC buffer (0.1% BSA, 2 mM EDTA in PBS), the single cell suspension was blocked with Fc block (anti-mouseCD16/CD32, 1:100 dilution, BioLegend; TrueStain Fcx; Cat. No. 101320), followed by washing and staining with Anti-CD45-PE (BD Pharmingen; cat. No. 553081), Anti-Ly6G-Pacific Blue (BioLegend; cat. No. 101224), and viability staining (invitrogen; Cat. No. 65-0864-14). The single-cell suspension was washed once more and spiked with CountBright™ Absolute Counting Beads (Invitrogen™; Cat. No. C36950). Samples were run on a BD LSR Fortessa (BD Biosciences, San Jose CA) using FC Diva software and analysed on FlowJo software (Ashland, OR). Once the doublets (by FSC-H vs. FSC-A) and dead cells (live vs. dead) were excluded, neutrophils were identified as CD45+Ly6G⁺ cells and further quantified using beads, lot-specific bead concentration, and the manufacturer's calculation guidelines.

### Tissue Lysate Preparation

For tissue lysate, tissue was dissected, washed with PBS and homogenized vigorously. Tissue was then added to RIPA Lysis and Extraction Buffer (Thermo Scientific™; Catalog number: 89900) and incubated for 30 minutes at 37° C. Afterwards the tissue suspension was sonicated for 2-5 min in rounds of 10 seconds at time at a power of 180 watts. Sample was kept on ice throughout the whole process. Protein levels were determined using Bradford Assay Dye-based protein detection (Thermofisher Scientific; Catalogue Number 23236) according to manufacturer protocol.

### Peripheral Blood and Plasma Analysis

Blood was collected from anesthetized mice via the retroorbital sinus into EDTA-coated capillary tubes and was analyzed by a Hemavet 950FS (Drew Scientific) for complete blood counts. Platelet-poor plasma was prepared immediately after blood collection by centrifuging anticoagulated whole blood for 5 minutes at 2300 g. Plasma supernatant was carefully removed and centrifuged again for 10 minutes at 16100 g to remove any remaining blood cells. Plasma samples were immediately stored at −80° C. until analysis.

### Determination of Plasma Tissue Protein Levels

Enzyme-linked immunosorbent assay (ELISA) was performed for Interleukin-10 (ELISA MAX™ Deluxe Set Mouse IL-1β; Catalogue Number 432616; BioLegend), Interleukin-6 (ELISA MAX™ Deluxe Set Mouse IL-6; Catalogue Number 431316; BioLegend), and B-Type Natriuretic Peptide (BNP) (RayBio® Mouse/Rat Brain Natriuretic Peptide EIA Kit; Catalog #: EIAM-BNP, EIAR-BNP; MyBioSource) to determine protein plasma or tissue lysate levels. Western Blot analysis was performed using anti-H4cit (Abcam; ab81797) and VWF (DakoCytomation; P0226) antibody. (Fukui, Gutch et al. 2022)

### Statistics

Values were tested for a Gaussian distribution using the D'Agostino-Pearson omnibus normality test with a 95% confidence level or Kolmogorov-Smirnov test. Continuous variables are presented as medians±lower and upper quartiles if they followed a non-Gaussian distribution and as means±SEM if they followed a Gaussian distribution. Non-normally distributed variables were tested using the Mann-Whitney U-test for unpaired analysis and the Wilcoxon matched-pairs side rank test for paired analysis. Normally-distributed values were tested using unpaired or paired Student's T-test. Differences between more than two groups were compared using Kruskal-Wallis test or ordinary one-way ANOVA respectively. Sample size calculation for the comparative analysis of WT DBA1 vs. DBA1 with CIA was estimated from a previous study using echocardiography data from C57BL/6 male yielding a n=12 mice in each group for 95% power at the 0.05 level of significance to detect a difference in E/A ratio assuming a mean (standard deviation) of 1.5 (0.2) for the control group and a mean (standard deviation) of 1.2 (0.2) for the CIA group. All figures are presented as mean±SEM. In all cases, a P<0.05 was considered statistically significant.

### Results

Mice with Collagen Induced Arthritis (CIA) Develop Diastolic Dysfunction and Myocardial Hypertrophy.

The CIA model in DBA/1 mice is the most widely used model to reproduce clinical symptoms of human RA. (Cho, Cho et al. 2007) In the present study, the DBA/1 strain mice were immunized two times with a bovine type II collagen (CII) emulsion in complete Freund's adjuvant on day 0 and then in incomplete Freund's adjuvant on day 21. (Fukui, Gutch et al. 2022) The first clinical signs of arthritis (ankle, paw swelling) became visible after the booster injection from day 25 (±2.3). After 8-weeks, DBA/1 J mice developed an average arthritis severity of 12 (±1.9) (FIG. 1A). Echocardiography at this time showed that CIA resulted in significantly reduced diastolic peak early to late filling velocity (E/A ratio) when compared to the healthy control indicative of impaired filling pressures (1.1±0.3 vs. 1.7±0.2; n=12; p<0.0001) with no difference in systolic left ventricular ejection fraction when compared to the healthy control mice (62+4% vs. 63 4%; n=12; p=0.6) (FIGS. 1B, 1C). Both, mice with arthritis and healthy controls had equal heart rate at the time of the measurement (n=12; p=0.7). As impaired filling pressures are due to impaired left ventricular relaxation/stiffness they are also reflected in changes to the cardiac cycle. Correspondingly, in mice with CIA it took longer for the pressure in the LV to drop below that of the left atrium delaying the left ventricular filling process, as reflected in a prolonged Isovolumetric Relaxation Time (IVRT) (19.1 [16.8-22.9] ms vs. 15.3 [14.3-15.8]ms; n=12; p=0,004). Similarly, the duration for equalizing the pressure difference between the left atrium and the left ventricle (E-wave deceleration time [DT]) was prolonged in mice with CIA when compared to the healthy control group (29+5 ms vs. 19±4 ms; n=12; p<0.0001). (FIG. 1E).

In addition to the impaired relaxation of the LV as determined by echocardiography we observed, that mice with CIA developed cardiac hypertrophy with markedly increased heart-weight to tibia-length ratio (11.9±0.7 mg/mm vs. 9.7±0.8 mg/mm; n=12; p<0.0001) (FIG. 1F).

Left Hypertrophic Ventricular Remodeling and Fibrosis are Present in Mice with CIA with Clinically Relevant Diastolic Dysfunction Mimicking Heart Failure in RA.

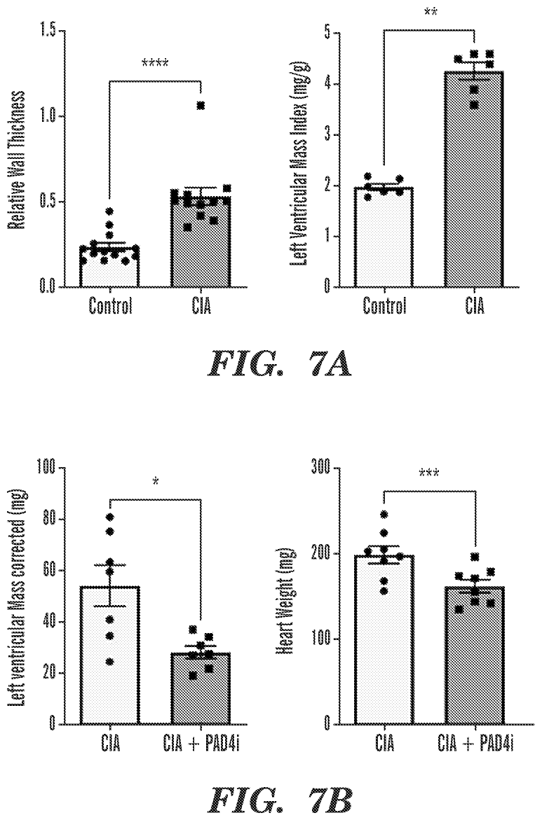

Hypertrophic myocardial remodeling in CIA mice was supported by an approximately 15% increase in cardiomyocyte cell size (n=5-6; p=0.03) as well as elevated measurements reflecting hypertrophic LV geometry such as an increased relative wall thickness (0.45 [0.4-0.5] vs. 0.28 [0.2-0.3]; n=12; p=0.004), a 75% increase in LVPWd (n=12; p<0.001) and an increased LV mass (60 [52-75]mg vs. 40 [35-57]mg; n=12 p=0.04), when compared to a healthy age-matched control group (FIGS. 2A, 7A). With increased relative wall thickness and left ventricular mass, the left ventricular hypertrophy in mice with CIA can be characterized as concentric. Histological analysis showed that myocardial hypertrophy was likely based on the increased size of cardiomyocytes and increased deposition of extracellular matrix (myocardial fibrosis). Mice with CIA showed increased myocardial fibrosis when compared to the healthy control both of the total heart sections (7.9±3% vs. 3.2±1.8%; n=8-9 p=0.001) and notably in the perivascular tissue (25.5±7.3% vs. 8.2±3.5%; n=10-12; p<0.0001) (FIGS. 2C, 2D). Immunofluorescent analysis showed elevated deposition of collagen type 1 in mice with CIA when compared to healthy controls (15 [11-21] % vs. 3 [1-11] %; n=5; p=0.03) in the LV (FIG. 2E). Taking into account the inherent limitations in the experimental setup (swelling of paws in mice with CIA), to test the cardiac symptoms of heart failure such as breathlessness and fatigue during exercise, the plasma levels of BNP, a clinically used biomarker of heart failure, was relied on to confirm presence of heart failure as described before (Wang, Sano et al. 2020). Mice with CIA had significantly increased BNP levels when compared to healthy control mice (104+19.9 pg/ml vs. 47.4+16.4 pg/ml; n=12; p<0,0001) (FIG. 2F). LV diastolic dysfunction with preserved ejection fraction and LV structural remodeling as seen in CIA are hallmarks of HF with preserved ejection fraction (HFpEF) a HF phenotype that is increasing in prevalence with ill-defined basic mechanisms. (Pfeffer, Shah et al. 2019, Shah, Borlaug et al. 2020)

Neutrophils are Activated Both in the Circulation and Myocardium of Mice with CIA.

Chronic neutrophil activation in CIA would be detrimental to the heart by promoting neutrophil infiltration in the myocardium with subsequent NET formation. Indeed, neutrophils isolated from mice with CIA were more preactivated/H3Cit⁺-positive (49 [34-62]% H3Cit⁺ cells vs. 1 [0-6] % H3Cit⁺ cells; n=4-6; p=0.009) with higher propensity for NETosis when compared to neutrophils isolated from blood of healthy controls (3 [0.7-3] % of cells vs. 0.2 [0-0.6] % of cells; n=4-6; p=0.03) (FIG. 3A). The chronic proinflammatory setting of arthritis was reflected by a higher neutrophil-to-lymphocyte ratio at indicated timepoints (FIG. 3B) throughout the 8-week experiment in the DBA/1 J mice with CIA when compared to healthy controls (n=6; Day25: p=0.002/Day40: p=0.002/Day56: p=0.004).

Indeed, FC analysis of heart tissue lysates showed higher counts of neutrophils in the myocardium of mice with CIA when compared to a healthy control group (8±4 cells/mg per LV tissue vs. 3±2 cells/mg per LV tissue: n=12; p=0.001) (FIG. 3C) also when compared to the total number of infiltrating CD45⁺ cells (n=12; p=0.001). Interestingly, there was no difference in the number of infiltrating CD45⁺ cells (n=12; p=0.4) (FIG. 3C).

To check for activated neutrophils in the process of NETosis in heart tissue, immunofluorescence analysis of heart sections of the LV was performed in order to search for H3Cit⁺ and Ly6G⁺ double positive cells. Indeed, mice with CIA showed a five-times higher number of H3Cit⁺ neutrophils when compared to healthy controls (212±73 double⁺ cells/mm² vs. 43±40 double⁺ cells/mm²; n=8 p<0.0001) (FIG. 3E). Corresponding with the higher levels of activated neutrophils, an IL-1β ELISA of heart tissue lysate of mice with CIA showed also higher values of IL-1β (1325+891.1 pg/ml vs. 549.8+274.4 pg/ml; n=12; p=0.032). This indicates, that the neutrophils are contributing to the heart IL-1β production (FIG. 3D).

Thromboinflammation in and Around the Cardiac Micro-Vessels is Present in Mice with CIA.

Next, it was asked where the activated neutrophils predominantly reside and hence exert their detrimental functions. Activated (H3Cit⁺) neutrophils in mice with CIA were found to be 3 times more likely to be located perivascularly than within the cardiac tissue (n=5; p=0.02) (FIG. 4E). At the same time, VWF staining was evaluated as a marker of endothelial activation. Increased deposition of VWF was observed in arthritic joints before and hypothesized that similar mechanisms could be driving neutrophil infiltration in the CIA heart as well. (Savchenko, Borissoff et al. 2014, Fukui, Gutch et al. 2022) Indeed, deposition of VWF was found in the cardiac vasculature of mice with CIA to be 5-fold elevated when compared to healthy controls (n=5; p<0.0001) (FIG. 4A). Given the prothrombotic propensity of VWF, this increase could drive micro thrombosis in the CIA model and as expected, mice with CIA showed 2 times more platelet aggregates per left ventricular section than healthy controls (n=6; p=0.03) (FIGS. 4C, 4D).

Treatment with an Orally Available PAD4 Inhibitor Preserves LV Diastolic Function in Mice with CIA.

To evaluate the role of PAD4 in neutrophil activation and trafficking in CIA a selective oral PAD4 inhibitor was used (JBI-589), and administered once daily starting after the onset of clinical RA at day 30 for 26 consecutive days. Mice were divided into 2 groups with comparable arthritis scoring. One was to be gavaged with the PAD4 inhibitor, the other with vehicle. A once daily administration was chosen as opposed to a twice daily regime which significantly inhibits RA (unpublished data), in the hope that this regime would leave residual arthritis disease. Indeed, while mice treated with JBI-589 had lower arthritis scores in the early period of the model (Day 36 [9±0.5 vs. 5±0.8; n=8; p, 0.001], day 44 [12 (11-13) vs. 9 (7-11); n=8; p=0.02]), there was no longer a difference in arthritis severity at the end point of the study at day 56 between treated and untreated mice (12 [±1] vs. 12 [±0.9]; p=0.5) (FIG. 5A). Despite comparable disease severity, treatment with the PAD4 inhibitor was able to mitigate adverse myocardial remodeling throughout the treatment period. Mice with CIA treated with JBI-589 for 26 consecutive days had preserved diastolic function as reflected by the E/A ratio being comparable to healthy mice and 40% higher than the placebo CIA group (n=8; p<0.0001). Correspondingly, the cardiac cycle was intact in JBI-589 treated mice, exhibiting normal and hence shorter IVRT (15 [14-16]ms vs. 22 [20-23]ms; n=8; p=0.008) and DT (21 [14-24]ms vs. 30 [28-33]ms; n=8; p=0.04) than mice with CIA treated with vehicle. There was no difference in systolic left ventricular function between mice with CIA treated with vehicle and mice with CIA treated with PAD4 inhibitor (n=8; p=0.09) (FIGS. 5C, 5E). Both groups had equal heart rate at the time of the measurement (n=8; p=0.1). Finally, treatment with JBI-589 significantly reduced plasma BNP levels in treated mice when compared to animals treated with vehicle (106.8+23.1 pg/ml vs. 55.4+14.0 pg/ml; n=8; p<0,0001) (FIG. 7B). Histological analysis suggested that the functional improvement was likely due to the prevention of myocardial hypertrophy and myocardial fibrosis. Mice treated with JBI-589 had lower heart-weight to tibia-length ratio (9 [±2]mg/mm vs. 11 [±2]mg/mm; p=0.004) and reduced left ventricular mass measured by echocardiography (33 [27-39]mg vs. 74 [43-94]mg; n=8; p=0.03) compared to mice treated with vehicle (FIG. 7B). Mice treated with PAD4 inhibitor had less total myocardial fibrotic tissue (4 [3-4] % vs. 10 [5-11] %; n=7-8; p=0.01) and 5 times lower collagen type 1 deposition in the LV then the vehicle treated control (n=5; p=0.02) (FIG. 5F).

### Inhibition of PAD4 Reduces Thromboinflammation by Decreasing Neutrophil Infiltration, Neutrophil H3Cit Expression, and Endothelial Activation.

Consistent with the results showing a decrease in myocardial fibrosis and lack of hypertrophy, JBI-589 treated mice had reduced numbers of neutrophils per mg LV heart tissue, determined by FC analysis (3 [2-9]cells/mg heart vs. 10 [6-14]cells/mg heart; n=8 p=0.03). Immunofluorescence respectively showed decreased cell density of H3Cit+ and Ly6G+ double positive cells in JBI-589 treated mice with CIA when compared to mice with CIA treated with vehicle (7 [±7]cells/mm² vs. 212 [±73]cells/mm²; n=7 p<0.0001) (FIG. 6A, 6B). There was no difference in the number of circulating neutrophils at any timepoint between the groups.

Consistent with the assumption, that VWF could be involved in neutrophil recruitment in mice with CIA, mice with CIA treated with JBI-589 had lower levels of deposited VWF when compared to mice with CIA treated with vehicle (0.3 [0.1-0.7] % vs. 3 [3-4] %; n=8; p=0.003) (FIG. 6C). Finally, mice with CIA treated with vehicle had significantly higher heart tissue levels of IL1-β when compared to mice treated with PAD4 inhibitor (938.6 [±610]pg/ml vs. 221.2 [±57.1]pg/ml; p=0,009) (FIG. 6D).

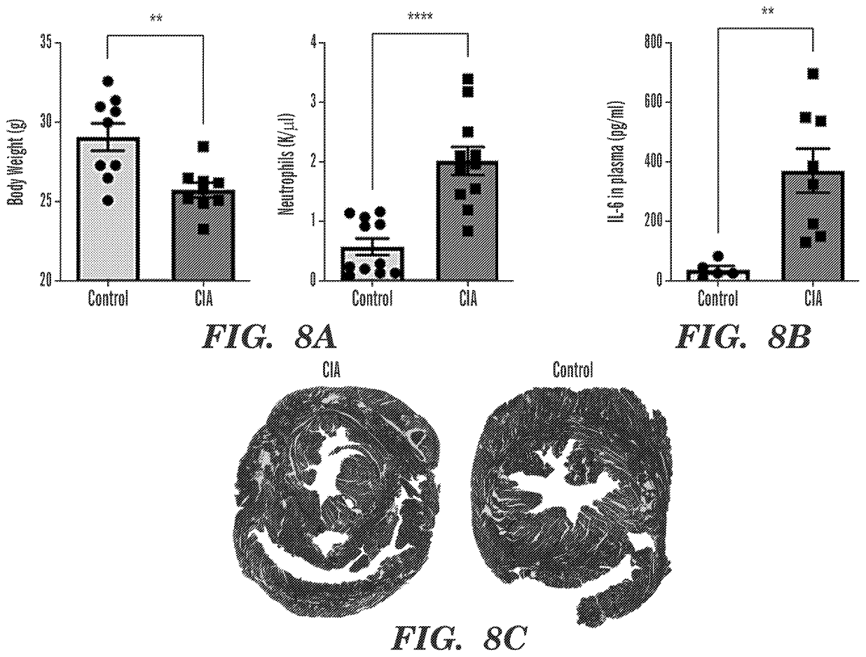

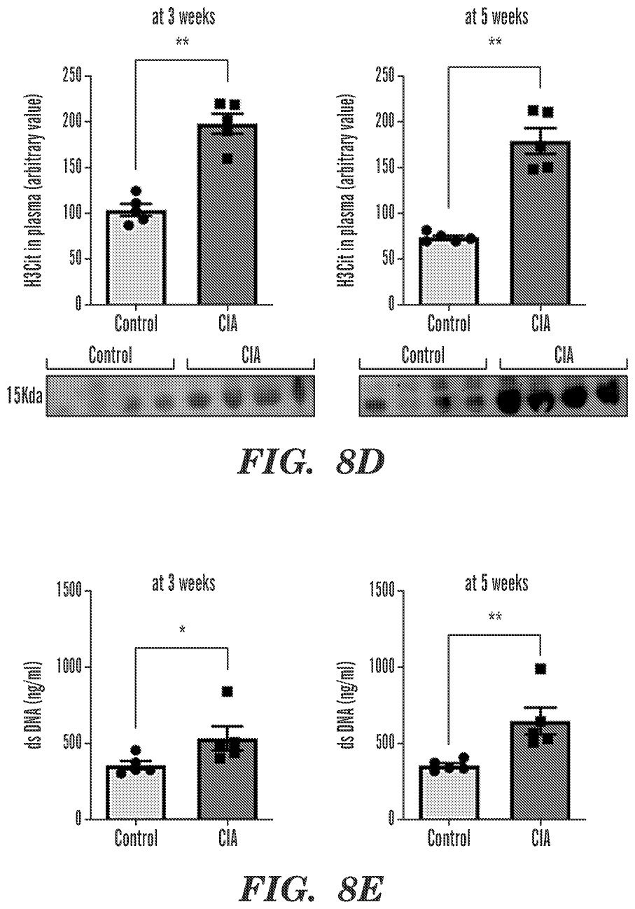

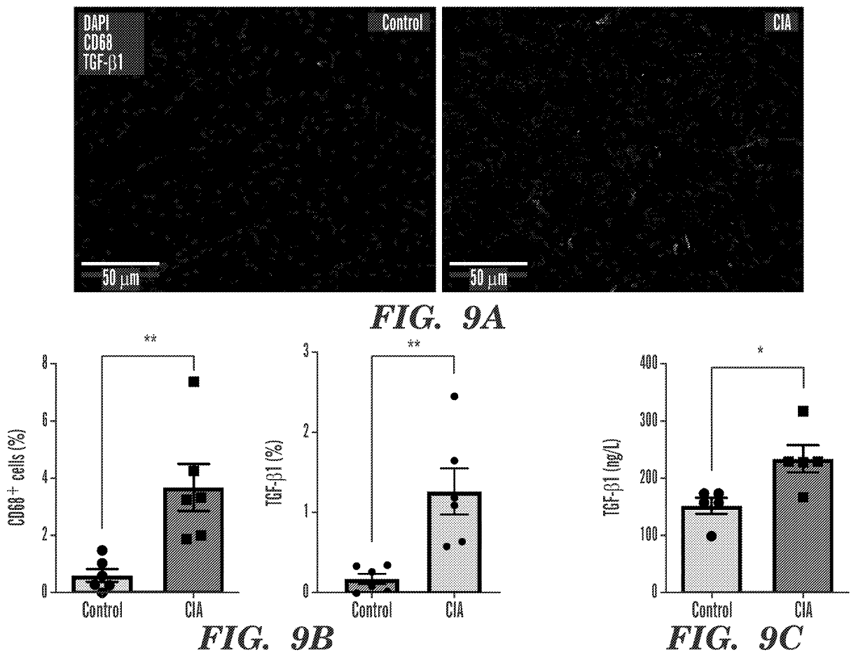

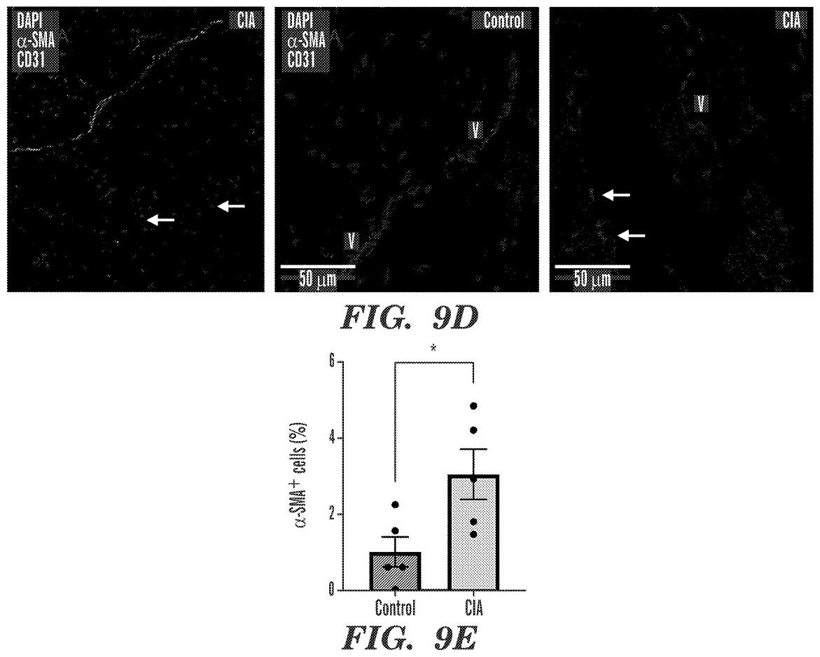

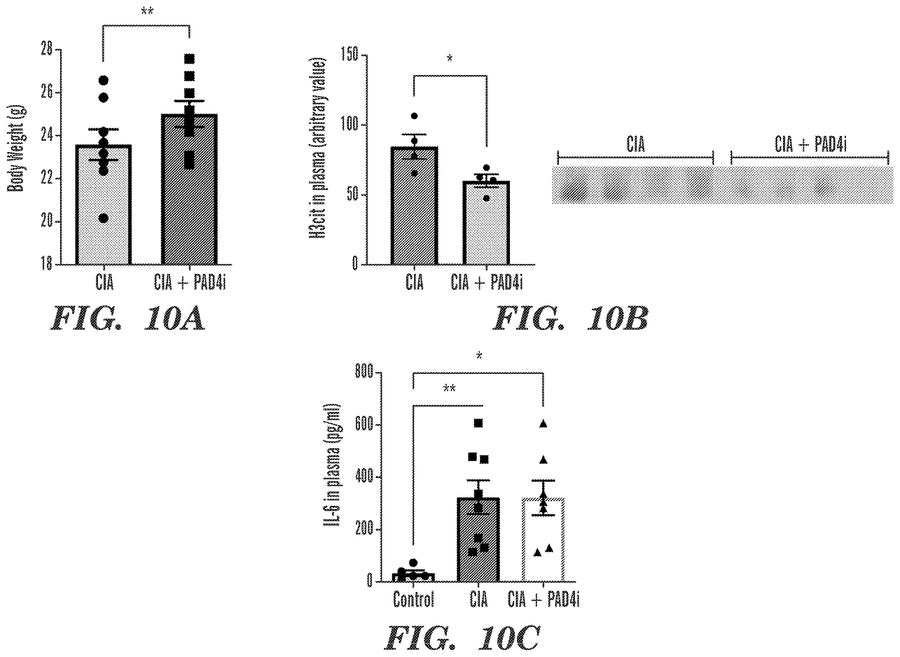

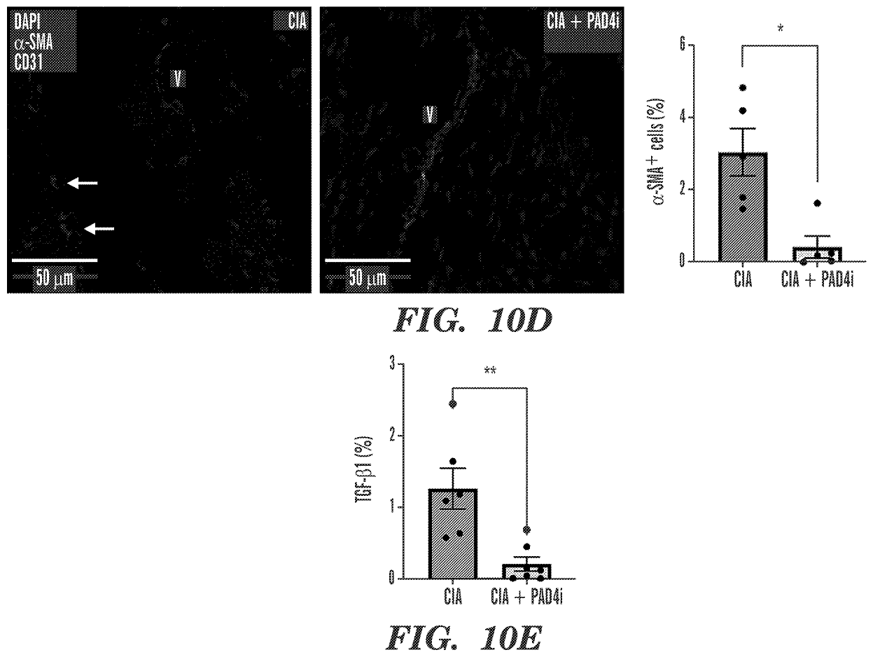

Thromboinflammation with elevated levels of inflammatory cytokines, release of NET biomarkers in plasma and decrease in health parameters were key components in CIA (FIGS. 8A-8E). Treatment with JBI-589 as expected reduced NET formation (n=4; p=0.03) but also improved health parameters such as body weight (n=8; p=0,009). Simultaneously, treatment with JBI-589 reduced the expression of pro-fibrotic markers. While mice with CIA showed increased levels of the profibrotic mediator transforming growth factor-β (TGF-β) in heart tissue (n=5; p=0.002) treatment with JBI-589 reduced deposition of TGF-β considerably (n=6; p=0.003) (FIGS. 9A-9E, FIGS. 10A-10D). The production of TGF-β has been implicated in driving the pro-fibrotic shift in heart tissue through fibroblast to myofibroblast phenoconversion. (Hulsmans, Sager et al. 2018, Frangogiannis 2020) Correspondingly, a 5-fold increase was observed in expression of alpha-smooth-muscle actin signal, a marker of activated myofibroblast (n=5; p=0.04) (FIGS. 9A-9E). Treatment with JBI-589 in turn reduced myofibroblast concentration (n=5; p=0.02) indicating, that PAD4 inhibition affects TGF-β production (FIGS. 10A-10E). Thus, inhibition of PAD4 does target both, thromboinflammation and profibrotic remodeling.

### Discussion

In this study, treatment with the new orally available PAD4 inhibitor JBI-589 was shown to be able to mitigate adverse myocardial remodeling in chronic inflammatory arthritis. (Deng, Lin et al. 2022, Gajendran, Fukui et al. 2023) Of particular significance, JBI-589 administration following onset of clinical arthritis decreased neutrophil recruitment to and deposition of NETs in the myocardium, and lowered cardiac levels of inflammatory and pro-fibrotic cytokines. This provides further evidence for an accentuated risk of collateral organ damage in chronic inflammatory arthritis and supports a prominent role of PAD4 in the underlying pathophysiological mechanism.

Aberrant activation of neutrophils through immune-complex-precipitation at the joints is characteristic for arthritis.(Fresneda Alarcon, McLaren et al. 2021) It is of interest to deepen the understanding on how, in this way, localized chronic inflammation triggers collateral damage. There is increasing evidence implicating neutrophils as being cellular mediators by locally delivering bioactive molecules such as Il-1β. (Sreejit, Nooti et al. 2022) Also, NETs derived from activated neutrophils may induce organ damage distant from the original injury. (Vats, Kaminski et al. 2022) The results support the concept, that PAD4, with its critical role in both the activation of neutrophils and extrusion of NETs, is involved in the mechanisms promoting collateral damage in arthritis, presumably through increased neutrophil recruitment and activation. There are several pathways through which PAD4 could influence neutrophil migration and activation, both directly and indirectly. For one thing, being the only PAD equipped with a nuclear localization sequence, PAD4 mediated citrullination is considered to be an important factor in the transcriptional regulation of the expression of various genes.(Li, Wang et al. 2010) It is therefore of interest that PAD4 plays a prominent role in in the regulation of CXCR2 (CXC-Motiv-Chemokinrezeptor 2) expression, a key cytokine receptor involved in neutrophil trafficking. (Metzemaekers, Gouwy et al. 2020) The results are also supported by recent findings in cancer involving PAD4 in the formation of distant premetastatic niches with inhibition of PAD4 via JBI-589 blocking neutrophil recruitment into the primary tumor and metastasis. (Deng, Lin et al. 2022) Furthermore, the role of PAD4 in NLRP3 inflammasome formation/activation was recently established. Activation of the multiprotein complex NLRP3 inflammasome promotes the release of Il-1β, an important endothelial activator active in leukocyte recruitment. (Kelley, Jeltema et al. 2019, Pyrillou, Burzynski et al. 2020) It is therefore likely that inhibition of PAD4 also affects the cytokine milieu of treated animals and consequently neutrophil migration. (Munzer, Negro et al. 2021, Fukui, Fukui et al. 2022)

What draws the activated neutrophils generated in the setting of arthritis to target the heart is not known. Alterations of the heart vasculature can direct organ specificity to autoimmune attacks. (Binstadt, Patel et al. 2006) Increased VWF release/deposition in the myocardial vasculature has been found in mice with arthritis and HF. In agreement, clinical data in humans demonstrates enhanced levels of endothelial adhesion molecules in the microvasculature of HF patients. (Franssen, Chen et al. 2016) Elevated PAD4 mediated neutrophil activation in arthritis could—in a vicious cycle—be a trigger for the release of additional VWF from Weibel Palade bodies facilitating the stabilization and endothelial anchorage of NET fibers fueling thromboinflammation. (Martinod und Wagner 2014, Sorvillo, Mizurini et al. 2019) Indeed, inhibiting PAD4 in the study resulted in reduced VWF surface expression in the myocardial vasculature. This could not only stem from reduced levels of PAD4-mediated NETosis in treated animals but from the role of extracellular PAD4 which, when released with NETs, is reducing the activity of ADAMTS13, a plasma metalloprotease that cleaves VWF. (Savchenko, Borissoff et al. 2014, Sorvillo, Mizurini et al. 2019)

There is evidence of elevated immune cell infiltration in the liver, kidney, and lung of arthritic mice. (Kwon, Lee et al. 2018, Shi, Shu et al. 2022) Inhibition of PAD4 could therefore not only be beneficial to the primary disease but several collateral comorbidities of RA as well. With its multiple functions both within different cell types and extracellularly, this detrimental effect of PAD4 is likely not limited to its influence on neutrophils and NETs alone. (Koushik, Joshi et al. 2017, Zhai, Wang et al. 2017) However, its crucial role in amplifying the neutrophil inflammatory response closely links PAD4 and neutrophils in arthritis and arthritis related HF. (Liu, Arfman et al. 2021)

Extensive hypertrophic concentric myocardial remodeling, increased cardiomyocyte cell size and increased fibrosis has been reported with collagen type 1 deposition in heart tissue of mice with arthritis. This resulted in clinically evident impaired diastolic left ventricular relaxation and stiffness and increased HF biomarker-levels.

Activation of PAD4 is closely linked to adverse myocardial remodeling. It has been shown, in aged mice as well as in an experimental model of cardiac fibrosis, that PAD4 deficiency protected hearts from fibrosis and ventricular remodeling. (Martinod, Witsch et al. 2017) In ex vivo models, myofibroblasts treated with NETs demonstrate increased connective tissue growth factor expression, collagen production, and proliferation/migration. (Chrysanthopoulou, Mitroulis et al. 2014) This is in line with our results showing that treatment with JBI-589 reduces myocardial fibrosis and collagen type 1 deposition in the myocardium, thus preventing adverse myocardial remodeling with resulting HF in arthritis.

It is difficult to quantify a potential contributing effect of CIA pathology in non-cardiac tissues which might be an additional factor for the development of HF (i.e., kidney failure or pulmonary hypertension). (Ahlers, Lowery et al. 2020) However, the fact that both treated and untreated arthritic mice had comparable clinical severity score in the end, renders it reasonable to argue that PAD4 activity is prominently involved in HF development in this late stage of arthritis. We believe that this could be due to increased PAD4-mediated neutrophil activation and NETosis in mice with CIA in cardiac tissue. The subsequent release and anchorage of VWF by NETs on the endothelial cell surface promotes platelet recruitment and secretion of profibrotic cytokines such as transforming growth factor β (TGF-β). (Mantovani, Cassatella et al. 2011, Horckmans, Ring et al. 2017) TGF-β in turn is critically involved in myofibroblast transdifferentiation driving cardiac fibrosis. (Dobaczewski, Chen et al. 2011) This is in line with the results showing increased deposition of myofibroblasts and TGF-β in cardiac tissue of mice with CIA. Downstream from PAD4 mediated neutrophil activation, NET components such as myeloperoxidase and histones have independently been shown to promote fibrosis and a direct adverse effect of inflammatory cytokines such as IL-1β on cardiomyocytes has been described possibly adding to the cardioprotective effect of PAD4 inhibition. (Hanna und Frangogiannis 2020, Yan, Li et al. 2023) The results show reduced levels of IL-1β in heart tissue of arthritic mice treated with JBI-589. This further supports a relationship between PAD4 and the NLRP3 inflammasome, the major source of IL--1β. (Munzer, Negro et al. 2021) It is reasonable to suspect that local IL-1β release in arthritis plays an integral role in attracting additional inflammatory cells and platelets. This again promotes cardiac release of TGF-β. (Chen, Norling et al. 2021)

Beyond that, NETs are recognized to attract functional tissue factor, containing microparticles, and to induce platelet activation and further thrombin generation propelling microvascular thrombosis. (Stakos, Kambas et al. 2015)

The impaired LV relaxation pattern combined with an increase in BNP and the preserved LV ejection fraction (FIG. 1) observed in CIA bears a striking resemblance to the HF subtype heart failure with preserved ejection fraction (HFpEF), a critical public health problem increasing in prevalence. (Dunlay, Roger et al. 2017, Pfeffer, Shah et al. 2019, Huusko, Tuominen et al. 2020, Virani, Alonso et al. 2021). The findings of this study support a key role of PAD4 and NETs in driving the pathophysiology of HFpEF that accompanies a variety of disorders with an inflammatory component and suggest CIA as an easy, accessible model to study HFpEF pathophysiology. (Lin, Fu et al. 2021, Schiattarella, Rodolico et al. 2021) It is possible, that an underlying chronic activation of neutrophils is a prominent etiological factor of HFpEF. However, this warrants further characterization of the model and a thorough examination taking into consideration the multiple sites of action/injury in CIA that could affect cardiac function outside neutrophil activation.

### CONCLUSION

In conclusion, evidence provided herein show a functional role of PAD4 and NETs in driving the inflammation-related heart failure in murine arthritis. The data support the role of inflammation-exposed activated neutrophils in cardiac IL-1β and TGF-β release and thus secondary organ damage. Inhibition of PAD4 could be a promising target to limit both the primary disease and the resulting thromboinflammation driving collateral damage in autoimmune disease.

### REFERENCES

Ahlers, M. J., B. D. Lowery, E. Farber-Eger, T. J. Wang, W. Bradham, M. J. Ormseth, C. P. Chung, C. M. Stein and D. K. Gupta (2020). “Heart Failure Risk Associated With Rheumatoid Arthritis-Related Chronic Inflammation.” J Am Heart Assoc 9(10): e014661.
Baoqi, Y., M. Dan, Z. Xingxing, Z. Xueqing, W. Yajing, X. Ke and Z. Liyun (2021). “Effect of Anti-Rheumatic Drugs on Cardiovascular Disease Events in Rheumatoid Arthritis.” Front Cardiovasc Med 8: 812631.
Binstadt, B. A., P. R. Patel, H. Alencar, P. A. Nigrovic, D. M. Lee, U. Mahmood, R. Weissleder, D. Mathis and C. Benoist (2006). “Particularities of the vasculature can promote the organ specificity of autoimmune attack.” Nat Immunol 7(3): 284-292.
Carrillo-Salinas, F. J., N. Ngwenyama, M. Anastasiou, K. Kaur and P. Alcaide (2019). “Heart Inflammation: Immune Cell Roles and Roads to the Heart.” Am J Pathol 189(8): 1482-1494.
Chen, J., L. V. Norling and D. Cooper (2021). “Cardiac Dysfunction in Rheumatoid Arthritis: The Role of Inflammation.” Cells 10(4).
Cho, Y. G., M. L. Cho, S. Y. Min and H. Y. Kim (2007). “Type II collagen autoimmunity in a mouse model of human rheumatoid arthritis.” Autoimmun Rev 7(1): 65-70.
Chrysanthopoulou, A., I. Mitroulis, E. Apostolidou, S. Arelaki, D. Mikroulis, T. Konstantinidis, E. Sivridis, M. Koffa, A. Giatromanolaki, D. T. Boumpas, K. Ritis and K. Kambas (2014). “Neutrophil extracellular traps promote differentiation and function of fibroblasts.” J Pathol 233(3): 294-307.
Deng, H., C. Lin, L. Garcia-Gerique, S. Fu, Z. Cruz, E. E. Bonner, M. Rosenwasser, S. Rajagopal, M. N. Sadhu, C. Gajendran, M. Zainuddin, R. Gosu, D. Sivanandhan, M. A. Shelef, B. Nam, D. T. Vogl, D. I. Gabrilovich and Y. Nefedova (2022). “A Novel Selective Inhibitor JBI-589 Targets PAD4-Mediated Neutrophil Migration to Suppress Tumor Progression.” Cancer Res 82(19): 3561-3572.
Dobaczewski, M., W. Chen and N. G. Frangogiannis (2011). “Transforming growth factor (TGF)-beta signaling in cardiac remodeling.” J Mol Cell Cardiol 51(4): 600-606.
Dunlay, S. M., V. L. Roger and M. M. Redfield (2017). “Epidemiology of heart failure with preserved ejection fraction.” Nat Rev Cardiol 14(10): 591-602.
Frangogiannis, N. (2020). “Transforming growth factor-beta in tissue fibrosis.” J Exp Med 217(3): e20190103.
Franssen, C., S. Chen, A. Unger, H. I. Korkmaz, G. W. De Keulenaer, C. Tschope, A. F. Leite-Moreira, R. Musters, H. W. Niessen, W. A. Linke, W. J. Paulus and N. Hamdani (2016). “Myocardial Microvascular Inflammatory Endothelial Activation in Heart Failure With Preserved Ejection Fraction.” JACC Heart Fail 4(4): 312-324.
Fresneda Alarcon, M., Z. McLaren and H. L. Wright (2021). “Neutrophils in the Pathogenesis of Rheumatoid Arthritis and Systemic Lupus Erythematosus: Same Foe Different M.O.” Front Immunol 12: 649693.
Fukui, S., S. Fukui, S. Van Bruggen, L. Shi, C. E. Sheehy, L. Chu and D. D. Wagner (2022). “NLRP3 inflammasome activation in neutrophils directs early inflammatory response in murine peritonitis.” Sci Rep 12(1): 21313.
Fukui, S., S. Gutch, S. Fukui, D. Cherpokova, K. Aymonnier, C. E. Sheehy, L. Chu and D. D. Wagner (2022). “The Prominent Role of Hematopoietic Peptidyl Arginine Deiminase 4 in Arthritis: Collagen- and Granulocyte Colony-Stimulating Factor-Induced Arthritis Model in C57BL/6 Mice.” Arthritis Rheumatol 74(7): 1139-1146.
Fukui, S., S. Gutch, S. Fukui, L. Chu and D. D. Wagner (2022). “Anti-inflammatory protective effect of ADAMTS-13 in murine arthritis models.” J Thromb Haemost 20(10): 2386-2393.
Gajendran, C., S. Fukui, N. M. Sadhu, M. Zainuddin, S. Rajagopal, R. Gosu, S. Gutch, S. Fukui, C. E. Sheehy, L. Chu, S. Vishwakarma, D. A. Jeyaraj, G. Hallur, D. D. Wagner and D. Sivanandhan (2023). “Alleviation of arthritis through prevention of neutrophil extracellular traps by an orally available inhibitor of protein arginine deiminase 4.” Sci Rep 13(1): 3189.
Hanna, A. and N. G. Frangogiannis (2020). “Inflammatory Cytokines and Chemokines as Therapeutic Targets in Heart Failure.” Cardiovasc Drugs Ther 34(6): 849-863.
Horckmans, M., L. Ring, J. Duchene, D. Santovito, M. J. Schloss, M. Drechsler, C. Weber, O. Soehnlein and S. Steffens (2017). “Neutrophils orchestrate post-myocardial infarction healing by polarizing macrophages towards a reparative phenotype.” Eur Heart J 38(3): 187-197.
Hulsmans, M., H. B. Sager, J. D. Roh, M. Valero-Munoz, N. E. Houstis, Y. Iwamoto, Y. Sun, R. M. Wilson, G. Wojtkiewicz, B. Tricot, M. T. Osborne, J. Hung, C. Vinegoni, K. Naxerova, D. E. Sosnovik, M. R. Zile, A. D. Bradshaw, R. Liao, A. Tawakol, R. Weissleder, A. Rosenzweig, F. K. Swirski, F. Sam and M. Nahrendorf (2018). “Cardiac macrophages promote diastolic dysfunction.” J Exp Med 215(2): 423-440.
Huusko, J., S. Tuominen, R. Studer, S. Corda, C. Proudfoot, M. Lassenius and H. Ukkonen (2020). “Recurrent hospitalizations are associated with increased mortality across the ejection fraction range in heart failure.” ESC Heart Fail 7(5): 2406-2417.
Kelley, N., D. Jeltema, Y. Duan and Y. He (2019). “The NLRP3 Inflammasome: An Overview of Mechanisms of Activation and Regulation.” Int J Mol Sci 20(13).
Koushik, S., N. Joshi, S. Nagaraju, S. Mahmood, K. Mudeenahally, R. Padmavathy, S. K. Jegatheesan, R. Mullangi and S. Rajagopal (2017). “PAD4: pathophysiology, current therapeutics and future perspective in rheumatoid arthritis.” Expert Opin Ther Targets 21(4): 433-447.
Kwon, O. C., E. J. Lee, E. J. Chang, J. Youn, B. Ghang, S. Hong, C. K. Lee, B. Yoo and Y. G. Kim (2018). “IL-17A(+)GM-CSF(+) Neutrophils Are the Major Infiltrating Cells in Interstitial Lung Disease in an Autoimmune Arthritis Model.” Front Immunol 9: 1544.
Li, P., D. Wang, H. Yao, P. Doret, G. Hao, Q. Shen, H. Qiu, X. Zhang, Y. Wang, G. Chen and Y. Wang (2010). “Coordination of PAD4 and HDAC2 in the regulation of p53-target gene expression.” Oncogene 29(21): 3153-3162.
Lin, Y., S. Fu, Y. Yao, Y. Li, Y. Zhao and L. Luo (2021). “Heart failure with preserved ejection fraction based on aging and comorbidities.” J Transl Med 19(1): 291.
Liu, X., T. Arfman, K. Wichapong, C. P. M. Reutelingsperger, J. Voorberg and G. A. F. Nicolaes (2021). “PAD4 takes charge during neutrophil activation: Impact of PAD4 mediated NET formation on immune-mediated disease.” J Thromb Haemost 19(7): 1607-1617.
Mantovani, A., M. A. Cassatella, C. Costantini and S. Jaillon (2011). “Neutrophils in the activation and regulation of innate and adaptive immunity.” Nat Rev Immunol 11(8): 519-531.
Martinod, K., M. Demers, T. A. Fuchs, S. L. Wong, A. Brill, M. Gallant, J. Hu, Y. Wang and D. D. Wagner (2013). “Neutrophil histone modification by peptidylarginine deiminase 4 is critical for deep vein thrombosis in mice.” Proc Natl Acad Sci USA 110(21): 8674-8679.
Martinod, K. and D. D. Wagner (2014). “Thrombosis: tangled up in NETs.” Blood 123(18): 2768-2776.
Martinod, K., T. Witsch, L. Erpenbeck, A. Savchenko, H. Hayashi, D. Cherpokova, M. Gallant, M. Mauler, S. M. Cifuni and D. D. Wagner (2017). “Peptidylarginine deiminase 4 promotes age-related organ fibrosis.” J Exp Med 214(2): 439-458.
Metzemaekers, M., M. Gouwy and P. Proost (2020). “Neutrophil chemoattractant receptors in health and disease: double-edged swords.” Cell Mol Immunol 17(5): 433-450.
Munzer, P., R. Negro, S. Fukui, L. di Meglio, K. Aymonnier, L. Chu, D. Cherpokova, S. Gutch, N. Sorvillo, L. Shi, V. G. Magupalli, A. N. R. Weber, R. E. Scharf, C. M. Waterman, H. Wu and D. D. Wagner (2021). “NLRP3 Inflammasome Assembly in Neutrophils Is Supported by PAD4 and Promotes NETosis Under Sterile Conditions.” Front Immunol 12: 683803.
Pfeffer, M. A., A. M. Shah and B. A. Borlaug (2019). “Heart Failure With Preserved Ejection Fraction In Perspective.” Circ Res 124(11): 1598-1617.
Pyrillou, K., L. C. Burzynski and M. C. H. Clarke (2020). “Alternative Pathways of IL-1 Activation, and Its Role in Health and Disease.” Front Immunol 11: 613170.
Roubille, C., V. Richer, T. Starnino, C. McCourt, A. McFarlane, P. Fleming, S. Siu, J. Kraft, C. Lynde, J. Pope, W. Gulliver, S. Keeling, J. Dutz, L. Bessette, R. Bissonnette and B. Haraoui (2015). “The effects of tumour necrosis factor inhibitors, methotrexate, non-steroidal anti-inflammatory drugs and corticosteroids on cardiovascular events in rheumatoid arthritis, psoriasis and psoriatic arthritis: a systematic review and meta-analysis.” Ann Rheum Dis 74(3): 480-489.
Savchenko, A. S., J. I. Borissoff, K. Martinod, S. F. De Meyer, M. Gallant, L. Erpenbeck, A. Brill, Y. Wang and D. D. Wagner (2014). “VWF-mediated leukocyte recruitment with chromatin decondensation by PAD4 increases myocardial ischemia/reperfusion injury in mice.” Blood 123(1): 141-148.
Schiattarella, G. G., D. Rodolico and J. A. Hill (2021). “Metabolic inflammation in heart failure with preserved ejection fraction.” Cardiovasc Res 117(2): 423-434.
Sebbag, M., S. Chapuy-Regaud, I. Auger, E. Petit-Texeira, C. Clavel, L. Nogueira, C. Vincent, F. Cornelis, J. Roudier and G. Serre (2004). “Clinical and pathophysiological significance of the autoimmune response to citrullinated proteins in rheumatoid arthritis.” Joint Bone Spine 71(6): 493-502.
Shah, S. J., B. A. Borlaug, D. W. Kitzman, A. D. McCulloch, B. C. Blaxall, R. Agarwal, J. A. Chirinos, S. Collins, R. C. Deo, M. T. Gladwin, H. Granzier, S. L. Hummel, D. A. Kass, M. M. Redfield, F. Sam, T. J. Wang, P. Desvigne-Nickens and B. B. Adhikari (2020). “Research Priorities for Heart Failure With Preserved Ejection Fraction: National Heart, Lung, and Blood Institute Working Group Summary.” Circulation 141(12): 1001-1026.
Shi, Y., J. Shu, Z. Ning, D. Fan, H. Shu, H. Zhao, L. Li, N. Zhao, C. Lu, A. Lu and X. He (2022). “Analysis of Hepatic Lipid Metabolism and Immune Function During the Development of Collagen-Induced Arthritis.” Front Immunol 13: 901697.
Song, W., J. Ye, N. Pan, C. Tan and M. Herrmann (2020). “Neutrophil Extracellular Traps Tied to Rheumatoid Arthritis: Points to Ponder.” Front Immunol 11: 578129.
Sorvillo, N., D. M. Mizurini, C. Coxon, K. Martinod, R. Tilvawala, D. Cherpokova, A. J. Salinger, R. J. Seward, C. Staudinger, E. Weerapana, N. I. Shapiro, C. E. Costello, P. R. Thompson and D. D. Wagner (2019). “Plasma Peptidylarginine Deiminase IV Promotes VWF-Platelet String Formation and Accelerates Thrombosis After Vessel Injury.” Circ Res 125(5): 507-519.
Sreejit, G., S. K. Nooti, R. M. Jaggers, B. Athmanathan, K. Ho Park, A. Al-Sharea, J. Johnson, A. Dahdah, M. K. S. Lee, J. Ma, A. J. Murphy and P. R. Nagareddy (2022). “Retention of the NLRP3 Inflammasome-Primed Neutrophils in the Bone Marrow Is Essential for Myocardial Infarction-Induced Granulopoiesis.” Circulation 145(1): 31-44.
Stakos, D. A., K. Kambas, T. Konstantinidis, I. Mitroulis, E. Apostolidou, S. Arelaki, V. Tsironidou, A. Giatromanolaki, P. Skendros, S. Konstantinides and K. Ritis (2015). “Expression of functional tissue factor by neutrophil extracellular traps in culprit artery of acute myocardial infarction.” Eur Heart J 36(22): 1405-1414.
Thiam, H. R., S. L. Wong, R. Qiu, M. Kittisopikul, A. Vahabikashi, A. E. Goldman, R. D. Goldman, D. D. Wagner and C. M. Waterman (2020). “NETosis proceeds by cytoskeleton and endomembrane disassembly and PAD4-mediated chromatin decondensation and nuclear envelope rupture.” Proc Natl Acad Sci USA 117(13): 7326-7337.
Vats, R., T. W. Kaminski, T. Brzoska, J. A. Leech, E. Tutuncuoglu, O. Katoch, J. Jonassaint, J. Tejero, E. M. Novelli, T. Pradhan-Sundd, M. T. Gladwin and P. Sundd (2022). “Liver-to-lung microembolic NETs promote gasdermin D-dependent inflammatory lung injury in sickle cell disease.” Blood 140(9): 1020-1037.
Virani, S. S., A. Alonso, H. J. Aparicio, E. J. Benjamin, M. S. Bittencourt, C. W. Callaway, A. P. Carson, A. M. Chamberlain, S. Cheng, F. N. Delling, M. S. V. Elkind, K. R. Evenson, J. F. Ferguson, D. K. Gupta, S. S. Khan, B. M. Kissela, K. L. Knutson, C. D. Lee, T. T. Lewis, J. Liu, M. S. Loop, P. L. Lutsey, J. Ma, J. Mackey, S. S. Martin, D. B. Matchar, M. E. Mussolino, S. D. Navaneethan, A. M. Perak, G. A. Roth, Z. Samad, G. M. Satou, E. B. Schroeder, S. H. Shah, C. M. Shay, A. Stokes, L. B. VanWagner, N. Y. Wang, C. W. Tsao, E. American Heart Association Council on, C. Prevention Statistics and S. Stroke Statistics (2021). “Heart Disease and Stroke Statistics-2021 Update: A Report From the American Heart Association.” Circulation 143(8): e254-e743.
Wang, Y., S. Sano, Y. Yura, Z. Ke, M. Sano, K. Oshima, H. Ogawa, K. Horitani, K. D. Min, E. Miura-Yura, A. Kour, M. A. Evans, M. A. Zuriaga, K. K. Hirschi, J. J. Fuster, E. M. Pietras and K. Walsh (2020). “Tet2-mediated clonal hematopoiesis in nonconditioned mice accelerates age-associated cardiac dysfunction.” JCI Insight 5(6).
Wong, S. L. and D. D. Wagner (2018). “Peptidylarginine deiminase 4: a nuclear button triggering neutrophil extracellular traps in inflammatory diseases and aging.” FASEB J: fj201800691R.
Yan, S., M. Li, B. Liu, Z. Ma and Q. Yang (2023). “Neutrophil extracellular traps and pulmonary fibrosis: an update.” J Inflamm (Lond) 20(1): 2.
Zhai, Q., L. Wang, P. Zhao and T. Li (2017). “Role of citrullination modification catalyzed by peptidylarginine deiminase 4 in gene transcriptional regulation.” Acta Biochim Biophys Sin (Shanghai) 49(7): 567-572.

What is claimed herein is:

1. A method of treating or preventing heart failure comprising administering a PAD4 inhibitor to a subject in need thereof.

2. The method of claim 1, wherein the subject in need thereof has been diagnosed with having heart failure.

3. The method of claim 1, wherein the subject in need thereof has been diagnosed with being at risk of having heart failure.

4. The method of claim 1, wherein the PAD4 inhibitor is an antibody reagent, an inhibitory nucleic acid, or a small molecule.

5. The method of any of claims 1-4, wherein the PAD4 inhibitor inhibits PAD4 expression and/or activity.

6. The method of claim 5, wherein PAD4 expression and/or activity is inhibited by at least 10%, 20%, 30%, 40%, 50%, 60%, 70%, 80%, 90%, 99% or more as compared to expression and/or activity prior to administration.

7. The method of claim 4, wherein the small molecule is JBI-589:

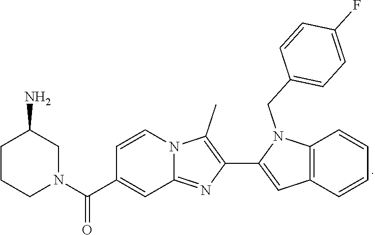 [SMILES: Cc1c(-c2cc3ccccc3n2Cc2ccc(F)cc2)nc2cc(C(=O)N3CCC[C@@H](N)C3)ccn12]

8. The method of claim 1, wherein the administering is selected from the group comprising topically, intravascularly, intravenously, intraarterially, intratumorally, intramuscularly, subcutaneously, intraperitoneally, intranasally, or orally.

9. The method of claim 1, wherein the heart failure comprises diastolic dysfunction, diastolic heart failure, and heart failure with preserved ejection fraction (HFpEF).

10. The method of claim 9, wherein the heart failure is HFpEF.

11. The method of claim 10, wherein the HFpEF arises as a result of autoimmune diseases, Diabetes Mellitus, hypertension, aging, and obesity.

12. The method of claim 1, further comprising the step, prior to administering, diagnosing the subject of having or at risk of having heart failure.

13. The method of claim 1, further comprising the step, prior to administering, receiving the results of an assay that diagnoses the subject of having or at risk of having heart failure.

14. The method of any one of claims 1-13, wherein the subject is a mammal.

15. The method of claim 14, wherein the mammal is human.

16. A method of treating or preventing heart failure, the method comprising administering a PAD4 inhibitor to a subject in need thereof, wherein the PAD4 inhibitor is JBI-589 or a derivative thereof.

17. A composition for the treatment or prevention of heart failure comprising a PAD4 inhibitor and a pharmaceutically acceptable carrier.

18. The composition of claim 17, wherein the PAD4 inhibitor is an antibody reagent, an inhibitory nucleic acid, or a small molecule.

19. The composition of claim 18, wherein the PAD4 inhibitor is JBI-589:

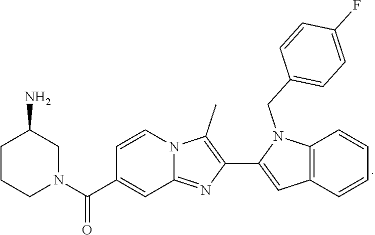 [SMILES: Cc1c(-c2cc3ccccc3n2Cc2ccc(F)cc2)nc2cc(C(=O)N3CCC[C@@H](N)C3)ccn12]

20. The composition of claim 17, wherein the heart failure comprises diastolic dysfunction, diastolic heart failure, and HFpEF.

21. The composition of claim 20, wherein the heart failure is HFpEF.

22. The composition of claim 21, wherein the HFpEF arises as a result of autoimmune diseases, Diabetes Mellitus, hypertension, aging, and obesity.

23. The composition of any one of claims 1-22, wherein the subject is a mammal.

24. The composition of claim 23, wherein the mammal is human.

25. A composition for treating or preventing heart failure, the composition comprising a PAD4 inhibitor, wherein the PAD4 inhibitor is JBI-589 or a derivative thereof.

26. Use of a composition comprising a PAD4 inhibitor for the treatment of prevention of heart failure, wherein the PAD4 inhibitor is JBI-589 or a derivative thereof.


SEQUENCE LISTING (3 sequences)

SEQ ID NO: 1 - DNA, 55808 bp, Homo sapiens
  source 1..55808: mol_type = genomic DNA
agccagagggacgagctagcccgacgatggcccaggggacattgatccgtgtgaccccagagcagcccacccatgccgtgtgtgtgctgggcaccttgactcagcttgacatctgcaggtaagaggggggccttctggggttttggaggcaggtcaggagatgctggatgacccagttctactgacacaggagcatgtgtttggcccaggctctaggctccagcctctgcagccactgccaggggagtagctggagagagaagaccccagcagccctgggaagtgccagtctcatggctgtgggttctttgccatgtacaagtgcaaattccccaggaacacacgacagataaattattatggggcaaaccaggccatccatctccctgtcttcatcgaggcaacagaagcttacagagagtctgtgacttgctcaaggtcacacagcacgtgtgccctggaagtaaggttagcccctgggcctcctggttcacagcacagtttgttaccccacatctcttagctaagcctccctcagtgggtgcataatagcgtgggtggggatggcgaaacggggctctgtggcggcagagactgaggtaggaagcctggacctgctgctggctagcaaggaggccttgggcaactgagcctcagttgcctcatctgtgaaatggggataatactcacatcatgggctggttataggaattcaatgtgaaaaaatgtgcagatgttggctgagtacttagcacagtggtgggcccatccttggtagtttttatttactcatttaacaaatgctgagtgccatgctctgtggcagggtctgggttaaacaagagagttgaggaatgctctcggggagtgtgcagggtcattgttgttattgtagaacaggaattaaaagaaattttaaaacgtgtaagcaaaaactcagttctatgttaaaaaaaaaaaaaacaattttccctggaaaaaaaaaaaaaaaagagctggagtcccttaaaaattaactgcctgtttttttgtggctagtgagccttattgctcctctttttccaggcattgtaaagaccctgtttctccagctgtgcagctgcaaggtcactagacagataaactcaagctgtaaaaatttttttccttaaaaagtaaaaaataatataatgcatgtctcaattaaataactgtctttgtttcttgcctctataatatgcttccccctgcacagatctacccccgccccatgaaatgcttaaaagataacttaactctttgttcagggctcagtcttttgaaatgttagcctgactgagccagtgaatctaaataataaatcctcctcaacccctcggtctctctgattcctaaattatcctgcaacaattgctgttgtcaggaataggagtggttgcccctggcttccccatcccagtctgggcatcggtggccctagaatggtgcccacaggagtgccggggacatttctttgctactgcctgagccccagcagccagagaacagcaggaaatgtcgggatggtctcccagggccacatggtggaggcaggagaccaagaagttggggagaaaaggaaaaagaaaatcacctgacatcatctgaggcgcccccaactcctctgagaatgggtgtgaaggaaatgacaggagctgaagctagcccagggcctctcccagcatgcccaggctagccctctacccagcgtccatgagtagtaatcaagatcacaagtgcttcctctggccctagagaggtgggtggacagctggtcagtgttgggcagagaggaaggcactgcccatttctcccagaaaggatggggactccggagttcacaggggcctctaaatccttcttttctcctccttcagccacacgttgagcctgtcactctatcccctgaacatctcacgtaacaatggctaatatttacatcacacttcctacattcaactcattcaatggccttgggaggcaggtaatgttgttattcccattttacaaaagaggaaacagaggctccaagaagttaagtaacccacctcaagccacacagctaataagtaggggagccaggactcaacccaggcagcctgactctaggaccctccattctccccatgggtctccgttccagaccttcctgtcacttcttatctgttcttaacatagccccagacagcgcctccctcccatctcctgagcagccatcggcctcttcctgaaccacacaggtgggagggccactccctcctcacctccgcccctcatattcatgcctccataactttgctcaagccattctctctgtccccaaagtcaggaacccgggaggcagaggttgcagtgagccaagattgggctagtgcactccagcctgggtgacagagtaagactctgtctcaaaaaaaaagaggcaaaaacacctaaccgagggtcacataaattaaaggtggcaagctgggcacggtggcttatgcctgtaatcccagcactttgagaggccaagatgggcaaatcacttgtggtcagaatttcgagaccagcctggccaacatggggaaaccccgtctctactaaaaatacaaaaaaattagacggggcgcaatggctcacatctgtaatcccagcactttgggaggccgaggcaggcggatcacaaggtcaggagatcgagaccatcctggctaacatggtgaaacgccgtctctactaaaaatacaaaaattagccgggcgtggtagcacgcagctatagtcccagctacttgggaggctgaggcaggagaatcgcttgaacccaggaggcggacgctgcagtgaaccgagattgtgccactgaactccagcctgggcaacacagtgagtctccgtctcaaaaaaaaaaaaaaaaattagatgggagtagtggcacatgcctgtaatctcaactactcaggaggctgaggcacaagagttgcttgaacctgggaggtggaggttgcagtgaggcaagattgcaccactgcactccagcaagggcaacagagtgagactctgtctcaaaaaaaaaaaagttaaaggtggcaaagccaggactgtctgaccctaaacccaccctgcttcgcagcccaccctgcctccaaatgctggggagtggactcccctgggcactgctccctgtgggcagagctcattcaacttccaaaggatttggtaaaggcctagaaggggtgggggtccaggaggtgccaactgcaaagggcagtgtgcattcagaagctgccatctatggtctctcttcttcctcccaaagtctccttcttcttttttttttttttaaaaacggagtctcgctctgtccagcccaggctggagtgcagtggcaagatcttggctcaccgcaagctccacctcccgggttcacgcaattctcctgcctcagcctccccagtagctgggactacaggcgcctgccaccatgcctggctaatttttttgtattttcagtagagacggtgtttcaccgtgttagccaggatggtctcgatctcctgacctcgtgatccacccgcctcggcctcccaaagtgctgggattacaggcttgagccaccgcgcccagccgggtttccttctgaatagaccagttggcatgacagtggggagcctggacaagtctgtttgtctctctgagcctcggtttacccatctgtaaaatgggaaaattaacaccccttcctgggttacggcgaagattgaattatataatgcacacaaagtgtctggcagaggggtcccctttccaatgcaaatcaaagaaaatgactaaagtgagtgtcaatcattttcagaggtttatttccaaggttaaggacgtgcccaggaaaagaatacagaaacaggaaaaatctgtagtccgtgctttttcctaaatgggtctgggacttcaatatttaaagggaaaagagtgggaattgggccggatgtggtggctcacgcctgtaatcccagcactttaggagaccaaggagggtggatcacctgaggtcaggagttcgaaaccagcctggacaacatggtgaaaccccatctctactaaaaatacaaaaattagccgggcgtggtggcagacgcttgtaatcccagctactcaggaagctgaggcaagagaatcacttgaacccaggaggcggaggttgcagtgagctgagattgcaccattgcactccaacctgggcaataagagtgaaactccgcctcaaaaaaaaaaaaaaaaaagtgggtattggggcaaagaggaagaaagaaaaaaaagatgggtagataagaggtaagtggttgcattgagtctttgatcagctttcactgaatacattttccgtgtgaggtggggagaggaatagtcacttatgccttgagctcagggaatctatattgtcacaaatgataaacatagagcaagggaagaaatcagatatgcatttgttgcaggtaagcagagggataactttgatttctgtcctttgttccatgcctgtgaagataagctattaacttacattgtcagggtgaaattcaacagaactggttcacgggaaagatcttagggctcacaaggaatttccttgtcggcaaattgtgagggagatatgtagcttttttttttttttttttaatctttgtagctatcttatttagaaacaaaatggggctgggcatggtggctcacgcctgtaatcccaacactttaggaggctgaggcgggcagatcatgaggtcaagggatggagaccatcctggccaacatggtgaaaccctgtctctactaaaaatacaaaaattagctgagcatggtggcgtgcgcctgtagtcccagctactcgcgagggtgaggcaagagaatcgcttgaacctgggaggcggaggttgcagtgagcagagatggcaccactgccctccagcctaggcgacagagcgagactctgcctcaaaaaaaatagaaagaaagaaacaatggggccaggcgtggtagctcatgcctgtaatcccaacactttgggaggctgaagtgggcagatcacttgaggttaggagttcaagaccagcctagccaacatggtgaaaccccatctctactaaaaatacaaaaacttagccaggcgtggtggtgcatgcctgtaatcccagctactcgggaggctgaggcacgagaattgcttgaacctgggaggcggaggctgcagtgagccaagatcatgccacggtactccagcctgggcgacagtgcaagaccttgtcaaaaaaaaaaaaaaaaaaaaaaaggaaaggaaggaaagaaggaaagaaggaaagaaagaaagaaaatgggaggcagatttgcatgacccagttcctagcttgactttccctttggcttagtgagtttggggtcctgagatttattttcctttgacagcagcgaggactgcatgagcctctgtgtgtgagagtggcagacactgaatgccaccctcatgggggtggttgatcaggagatggcggatgctggagttggaggaatgatgtcactgtctggggctcaagctcccagtgctggctgtatggagggtgttggaagttacaagaagattcagctctggatggtcttatgataacacatgtgggtcacacgcttgagggcatgcacacacctggtgtgaacccccagatgtttatgaaagttccttgtttaggagctgagaacatctggaacacagccagaggaagcacactcatcttagaatcgaatcccagtggcccagttcccagctggcccttacctctctgagcctcagtttccccatctagcagagagagtggctataaaggtgccagacccacccgtttgtgcccttctcccttcccctcttgaggaagccacatgggacagagggggctgaggcggacctagcccctcggtccacctgctgtgtgcccctcggcctccattgtcttttgagccccttattcatcttcttggtggaccagacacagggcgtgtccctctcctgtccccatcctctgaccacagtgcccaggcagttggaaggcctggcttatcactgaggctttgagagaatgaaggaaaacaacagcacacagaaccaagagaatgagcctccaggctttggcagagaaacaagagccttggttaagttttggttcggccaggtaggtgtatcataaatggaggcacctgagcagaaatgcaccagagccactggggcatctcctggccgggaactgccccctgggctgagctgtctgctgcttcatttattgttattattagtagccgtcccatactgagcagttaccacatgccgggccctccaaaccttgcaaccacccaggaagcaggtcgacgtgattcccattttacagagaaggaagctgaggcccaggagaggtcagtggctaggccaaggtcacagagcctgtcagaagcagcccaaggtcattgcctctctcactcccttcgaggctgcctgctgctcctggcctggcctgggagacgtttccccggggcagctgaaccactccaccccagctggtctgcaggagagtatctgatcccaaactgctccaaccagctggtgatggctgagtgcaaaaccacaagcacatgctgtgccctggctggctggtgtagacagactggcctgctgtggtatgtggtgtgttgggaattcgtatgttccattcacactgaccctggcaatctccccgaggcccaggtcagtgccacccggggtgatgacaacccagccatcctgctccccacccccgtcagcatgggaaccaggctgggagcaggtgggaaagccaccacctgagctgaggggacaggcagggacagccatggcttgagtttagcagatgtagaatgacgtccttgtcaaggaggactggggggagtccacaagggaaccccagattttccaccttgtcccatccacctttgtcctcagcacaaggtctggtacacagtgaggggtgagtgagtgtgtggtgattgattgattgattgaatgaacgaatgaaggaatgagtgtgtgttttaagggaaactatgcctgtcacacaggaggtgctatttaagtgtttgttgagtgaatgaaagaaagttgagagcaggaagggtgtcattcctctttgcatcccctataatgtgtgggacgtacttggcactcagctatcatgaaatctttttgcaacaagagcaggcggaggataaaagggaaagagagaaaaattgagagggatatggagagaaagagaacatactcaaaaacattccaccatcctgacatcagccaccaccatcccttacagcgcaggagcccctacatcccaggccactgggacatggttttttgcttccatggcccttcgggaaggacccccaacactggctcctttggtcaaagactgcagtgtgagttcaacaggagagagagaaggggaggagcaagtaaaagaaaactttctaaaaatcggtgcctgctgtgacccacacccttttctcataccttcccttgtagccctgggaaagaggaattcttcctttccatttttggagaaaattctgggatcctacactgggttcccattcaggggctgctctctcaacccccagtccccacccacggtgacctcacagcccccagggctacacctgtcacttgaacctggctgcctccctgatgacccacagctctgccctgacccctcctctctcggcccctatccacagggtgcctccacccagatggcccggcaaaaacaaaaccagcctggctccctgccttttcctcctcctgatgaggaggctgccacccccatcttcccatttatgtcgataacactcctggctggatgcagtggctcacgcctataatcccaacactttgggaggccaaggtgggctgatcacatgaggccagaagtttgagaccagcctggtcaacatggcgaaaccccgtctctactaaaaatacaaaaattagctgggcatggtggtgcacgtctgtagtctcagctactaaggaggctgaggcgggagaatcatttgaacctgtgaggtggaggttgctgtgagccgagatcacaccactgcactccagcctgggtgacagagcaagactctgtctcaaaaataaataaatagccaggcgcggtggctcacgcctgtaatcccagcactttgggaggctgaggtgggcagatcacgaggtcaggagatcgagaccatcctggctaacacagtgaaaccccgtctctactaaaaatacaaaaaaattagccaggcctggtggcgggcgcctgtagtcccagctacttgggaggctgaggcaggagaatggcatgaacctgggaggtggagcttgcagtgagccaagatcgcaccactgcactcgagcctgggagacagagtgagactccgtctcaaaataaataaataaataaattaattaattaattaattaattaaaataaataaataaataaaataacacccctgcccaaaaacaacccccccacccgtctcccctgccccgcgcccagcttctatctgtagctgcatttcagccgggcattcagtagaagctgtgggactgcatgactggggcagagcctcagtgtgctcatctgtaaaatgggaagagggatgcctgcctcacagtagcaagacctcatagtgctgccgtgaatgttcagccgatgagacagtgtgtgtagaacgccaggctcacagtagctctcaacagataccagtgtatgcattctctccagcctcagtttcttcatttgtagaaggggaactccaatagtacgtatgtcgcagagttgtcatggagatgcctgtagagatggggtttagccatgttgcccaggctggtctcaaacttatggactcaagcaatccacttgcctcagcttcccaaagtgctagggttacaggcatgagccaccatgcctggctgtgtgtatggcttttctgcataagaaaatagcaataacaatgcacgccacagacataatgccgacggagtctcgctcttgtcgcctaggctggagtgcaatggtgagatcttggctcactgcaatctccacctcccaggttcaagcaattctcctgcctcagcctcctgagtagctgggattacagacacccaccaccacacctggctaatttttttgtatttttagtacagacggggattcaccatgttggccaggctggtctcgaactcctgacctcgtgatccgcccgtctcggcctcccaaagtgctgggattacaggcttgagccaccatgcctggccatcataatgctttatttattctgtcctgtgcatcatggcatggttccctcagattccactcaaaaatcagggtaattcagcaaactcgtattagcatcttctttggcagtggttctcacacttgagtgtgtatcaaaagcatctggagagcgtgttaaaacacagattgctgccgggcactgtggcttgcgcctgtaatcccagcactttgggaggccaaggcaggaggatcacttgaggccaggagttcgagaccagcctgggaaacatactgagactctcatctctataaaaatgttttttaaattagctggatgtggtagtgtgtgcctgtagtcccagctactcaggaggctgaggccggaggattgcttgagcccaggagttcaagtctgccgtgagccatgattgtgcctctgcattccagcaagacctcctctcagaaaaaccaaaacaaacaaaaaacacacacttctgggccccactcctagagtttccaactcagtaagtctgagtggagccccagaatctgcatttttaacaaattccccagtgaggcagatgctgctgttccgaagaccacactttgggaagcattggtctttaggaagtgctggatgctgggcacagcagagtggcaccaatgcagagaaggaggtagtcgtgatgtggcaggaatggcaccaggctgggctgcaagaggacccagacctgccattatcagccagcacccagaaacgtcactttgcctctctgaacctgttttctcatctgtaaaagaataacaatggctcactcataggatgattgtgaaaacaaaggttaatggccacacagagaaaggtgactagcttgtcatctgctgcaggcatgtgaacaccaactcaggcaacagtgtcaggacgggagaaggtgccaagaccgaaatgtcactgaggatgacagaaattctaaatgtcaaactcccaaacggaggcgggaagaataacctgactgctgagtgtgtcttaatgtcttgatcagacttcctgtgactttgagctctgaataaacatttttatattttcaccttttgccgttcatatgcacacaaggcgaaaattggcatgctgattttttttctttttttttttttgagatggagtctcgctctatcacccaggctggagtgcagtggcgcgatcttggctcactgcaaactccgcctcccgggttcatgccattctcttgcctcagcctcccgagtagctgggactacaggcacccgccacaactcccggctaatttttttttgtatttttaatagagatggggtttcaccatgttagccaggatggtctcgatctcctgaccttgtgatccacccgcctcggcctcccaaagtgctgggattacaggcgggagccactgcgcccagcctttttttttcctttttgtggagacagggtctagctattttgcccaggctggtagaaagcagtcctcccacctcagcctcccaaaatgctgggattacaggcgggagccatcgtgcctggccagtatactaacttttaaaaaaatctactaccacatgccccttgcttcttcctttttcttaggaagatgctgattttggttcaagtgtttttttgattgtctgccacttttccagacctcggccatcatatcactcaagagtacagggttttacctaggatcaagagagagacagaggcatggcgtggtggctcatgtctgtaatcccagcactttggaaggccaagatgggaggatcccttgagcccaggagttcaagaccagtctggagataaggagactccatctctacaaacaataattattttaaaattagccaggggtgatggcatgtacctgtggtcccagctactcaggaggctgaggtggaagggtcacttcagcctgggaggttgagttgcagtgcactgtgcttaaaaaaagagagagagagacattcaaagacagagggtgtattagtttcctatttgttaaaagaaacacaagacaaattaaatttaatagcttagtggagcaaagaaccattagtgaatcaggcagccccttttacccaaaccagaataggttcagaaagactcaggagctgccacatggttgaacaatatttacggacagaaaaacgaaagtgacccacagagcacggaagcgaggctcagaaacagctggattgactacagctcggcatcggcctcatttgaacatggtttgaacagttggctgcctgagatcagccaaaacagtgcttggtataaaagtaggtcacgggctgtttacacctccagttaggttacagtttactatgtacgaaaaaacctttcagccgaacttaaaatacctaaggaggcagctgtggggtaaacttaatataacacattgctgctgtaacaaatcactacaaacttagtgacttcaaacagcacaaacttattacttaccagtttgcaagatcagaagtccaaaatatgaatcttacttggctaaaatcaaggtgtcggcaaggctgtattccttctggaagctccaggggcaaacccattccttgcagcttctagaggtgcccgcattccttggcttgtggccccttccagcaacagcatcactctgatctctgctgccatcaccacagtttccctgactcagaccctcctgcctccctcttataaagacctttgtattactggaaagtggtcccaatccagaccccaaaagagggttcttgaattttgcctgagaaagaattcaaggtgaatccataaagtgaaagcaagtttattaagaacgcaaaggaataaaagaatggctactccatagacagagcagtcccaaaggctgctggttgcccatttttatggttatttcttgattatatgctaaacaaggggtggattattcatgagttttcagggtaaagggtgggcaattccctgaactgagggttcctccactttttagaccatatagggtaacttccaagcgttgccatagtatccgtaaactgtcatgacgctggtgggagtgtcttttagcatgctaatgtattataattagtgtataacgagcagtgaggacgaccagaagtcactctcgtcatcgtcttggttttggtggggtttggccagcttctttactgtcacctgttttatcagcaaggtctttatgacctgtgtcttgtgctgacctcctatctcatcctgtgacttagaatgcctaacttactgggaatgcagcccagcagatctcagccttattttatgcaacccctattcaagatggagttgctctggttcaaatgcctctgacacttgtggttacattgggcccatctggctaatctaggataatctccccatctcaagatcgttaacttaatcacatttgcaagtttccttttctgtgtaaggtaacatcttcacaggtttgggggactagggtgtggtcatctttgggagaccattgttctgcctaccacagagggctgatctcattttgaggaagaaatcccttttggttggagtgataaagggaggattcgatggcagagaacccactgagatgggcctttggggctggattggattctgcaagaaggatgtgggaagggagagccctccagatgaggggcaggtgagaagcaaagcccgggagatgggaaagtggggggcaggtgcagggaactgtgggccattcagtctgaccatagcacagggagagcagcacgggggaccactgggggccaggccagggcccaccctcactctctttatgaagcagagatccaatatacctaactcttgagtttgggaaagtgacttcacctctctgtgcctcagtttactcatctgtaaaatgaacagtacccgccttacaggctagtggtgagaaccgaattaattaacgtgtggaaagaatgtgaacagtgcctggagcagagtaaattgcctgtaagtaattgctgtcatttaagaatgtgaaattgacttcgtgagggtagaatgagagcccaagccagggtgggcaggagatggaatgaaatctcctcttctgtctacttcagacctcttaggttcttcaaaggctatgtcaaattccatctcctgaaggatcaacatcagaatgttagtgattgtcttgtaaatcaatgaaaatgaccaagatgagtgtcaatcattttagaaggtttatttgccaaagttaaggacccgtgtctgggaggcaggtccatgcccttctctgaagatgattttgaggccttggatatttaaaggagaaagggcggataatggaagaagatgaagaaatttttttaatgtgtgggtggataagagacaaacagttgcatccttttgagtctttgatcagccttttactgaaaacaccattttcgtgtgaggcaaggtagaggagtagtcacttatgcattcatctagctcagtgaatctgcatttttacatgagattacataaatatagcggagaggaagcaattacatatgcatttgtctcaggggagcagagggatgactttgagttctgtcctttgtcctgtacctgtaaagataagctctgtcctttgccctgtacctgtaaagataagctatcagctgggcgtggtggctcatgcctgtaatcccaacactttgggaggccaaggcgggtggatcacctgaggtcaggagtttgagaccagcctgaccaatatggtgaaaccctatctctactaaaaatacaaaaaattagccgggagtggtggcatgcacctgtagtcccagctacttgggaggctgagacaggagaattgcttgaacctgggaggcggaggctgcggtgagctgagattgcaccactgcactctagcctgggtgacagagtgagactccatccccccccaaaaaaaaagataagctataaatgtacaattgctagggagaaattcaacagaactgttttagggtaaagatctttggggcccacagggaatttccttgtgggccaattgtgagggaagtatgtagctttttatctttatagttattttatttatgaaccatgagaggcaggtttgagggacccagttcccaggactcttccctttggcttagtgagtttggggtcccaagatgtattttcctttcacagtctgtaagtggtaagattcattttcttctaggcaatttatgtcttcttccctcccttccttctttctttccttcttctttctctttttctctctttctccccttctccccctcccacttcttctttcttctggtatactgtattagctatctatactgtgtaacaaattgtcccaaaacttagtggctcaaaatgataacatttattgtatcataatttctataggttagcaatccagtatggtttagctgggttctctacttcaaggtttctcataatcaaggtattggtagagccgtagtcatgctaaggctccaccagggaaggtgacacttccaagcccactcatatggctgttggtaggattcagggccttgcagggtgttggaacctcagttcctccctggctgttggctggaagcctccctcagtttcctgccacattggcctctccatagctcacaaaatggcagcttgcttcatcaaggtgagcaaggcaagagacaggcagagagagaggcagagagagagacagagagagaattctattgtaaccttatgacagaagtgatattccagcacttttacttccttggttagaaggaagtcagtaggtccagcccatactcaaggggagagaattacacaagggcatgaacagcagagacgggaattactggggaccatcttagaagtctgtccaccatatacttttcctgtagtttcctaattctttataatgaatacatataactcttaatatagagacaaatattcaatacaaagataaaaatagaaaatgtaatcctaagccaggtactgtgattcatgcctgtaatcccagcactttgggaggccaaggcagcagatcacttaagtctaggggttcaagactagcctgcatgacatggcaaaaccccatctctacaaaaatacaaaaatcagctgggtgtgatggtgcacacctgtagtcctggctacttgggaggctgagatgagaggctagcttaagcccaggaggcagaggttgcagtgagctgagatcacaccactacactccagcctgggcaacagagatctcacctcaaaaaacaagcaaacaaacaacaacaacaacaaaaaaaacacaaacaaacaaaaaacacatgtaatcctaagtgtttctgtgaccttatgtggacggagttctgcctctctagggattgtccactgtgaatccactataatttattagctattcttttgtgggtatttagaccatgtttcattgttcacattacagaaagggctgctctgaactttcttgaacatgtctcctagtgaacacatgaaagagattctccagagtatctgcctagaagtcaaattgctgggtttggggctggctaataacaccaagttgggttttttttgtttttttttttttgcaaaatggttaatttcacactccagtaagcagtgtgttagagtttctgttgaatcgtgtcatttatagtgttatttttcaccaattgagtgggtataaaaccctatttctttatggtgttaaccttttcattctcttacttgttagtaaggtggagtgcctcttcatgttttcatgttgatttgcctaagttctttaaatagtctgcatgctaagcctttgttggttttatgttgtgaatatattctccttacattgtgcttgtctttttactttatattgtcttttgattaacagaatttattttcttttttaatttttaaattgatgttaattttttaaaaaatagaaacagggtctcactatgttgccctggctggccttgaactcctggctcaagtgatcttcccaccttggcctcccaaagcactgggattaaaggtgtgagccactgcacctggcctatttatttacatctagttgaattttttgatcttttccttcaggtttactggttttgagtcttgtttcagagaaagcataaatatgttatcctgtatcctcttctagagattcaaagttctgactttcacatttaagtctttatctccctccagctgatttttgtgtatggtgtgaggctgggatcaatttaattatttttccaatttagtattttctatatgaatgagcaattgctccagcaacatttatgatatagtctctactacctccagccatctgcaagcctatttctgtcatagatcaagttgtatgtcctttaagttttatccttcctttgtcttggcctgagccattaaaaccccatcttaagttccatggctttataggaagtcttatacctggttgagaaagtgcctcaccttttactactttcctttacaagtaccttgaccaacttagttcctggatttctcgtgtagatttaaaaatcaacttgtcaagttccataagaaaccctaaaattccattgaatatataaatccattggagagaattcatacctttacggcaacaaggcttcctaaccatatacatagtatttctatttatttggatcttctctaatggctttcaataaagtttgaatattttcttcataaaaatcttacacattttgctagatttatcctaggtactttacagtattttttccttttgtaaattacatcagttttttaaaaattgttttcttcttaaagaaatggtttgctagaaaaaaagaaaaatatttgtttcctatttattgctgatgtatagaaatgtaatttattttttcctctttactttattagtcaccttattaaacttttgtctttttatttatttttaatttttaaaaaagtttaattcactcttttggagtttgcagacaactgtattttctgtcaatattggcagtttttttcttacttttcactctttatatcttttactttatgtctttttttctcttgctgcagtgcttagaatcatccacacaatgttaaaaagatgtgactttttgatcctaccacgttcctgatttttttttttttaaacagagtctcactctgttacccaggctggagtgcaatggcacaatcttgactcactgcaacctctgcctcccaggttcaaacgattctcatgcctcagcctccccagtagctgagattacagacgtgcactacccagttaatttttgtatttttagtagagacagggttttgccacgttggccaggctggtctccaactcccgacctcatgggatctgcccacctcagcttcccaaagttctaggattacaggcatgagccactgcacccagccccatgtttctgactttaaagggaaggttttcacattttccctgtcaaacaggacgttctctgtgggtcttttgttaaccttttatcaactatctacagaaaaaaaaaaatctgctttatcttcagtaatatttcttttgctttcctaatattttttctcttgatcaatgttgccagagatttgtcagttttattggtctattctaagaaccaacttttggctttattgatttgttttattatttcacagatttcttccaactttattttattttgggattttacattacattttctttttctattaattttcagcttttactttctttttaaaaatataaacatttaaggcataaagtatcacttttacatctccccaaaagttttgaaaatagtattttagttattgctcagtcctaagtattattacttaatttccattattaattgtcctttgacttgtaagtaattgagatgtctggttttaaatttccaaatgtatggtgcttttaaaaacatactttatactagtgacttctaacttagttacattatgatcagagactctgctacccatatattacctatgctttaatgtttgtcacactgtcattgacttgctttatggccaattacatagtcacttttttaaatgacccatgacccgtgtgtgctttgcttgggaaaatgtatatttgctacttgtgggatacacaattctatatatatcgactaaatcaactttgttaattatgttagcaaaatctgtatcattacttaatttttatctgcttaatctattgttgattgagaaagaagagttgaaatcttcaactctgatggtggttttgtcaacttcttttccctatgaatgtatttctattaatgtttgccttatatgcttttttttttttttttgagacacagtctcaccctctcactctgtcacccaggctggggtacagtggtgcaatattggctcactgcaacctctgcctcccaggctcaagtgatcttcccacctctgcctcctgagtagctaggactacacgtgcatgccacagtgcctggctaattattttgtattttttgtagagatggggttttaccatattgctcaggctggtcttaaactcctgggctcaggcgatctgcccaccttggcctcccaatgtgttggtatgagccaccatgcccagccttgctttatatatgttgaggctcttattagatgcatacaagtttagaatatcttctaggtaagttaaaccgtatatcattaggttgtaatcctctctattgataccaatgtgttttgtctaataattcattcttttgatattaatatatccatcccagctctctttgagttagcatttgcttaatgtatcttttatcctttttaaaaatttttcaacttttctggatcgttatattttagatgtgtttcttataatcagtatattactggattttgtttatttaatgaaaactctcttttaactggcaatattaatcctgttatatttattttgattatttactttttttttttttgagatggagtctcgctttgtcacccaggctggagtacagtggcgtgatctcaggttactgcaacctctgcctctcgggttcaagcaattctcctgcctcagcctcccaagtggctgagattacaggcacgtacaaccatgcccagctaatttttgtatttttttttagtagagactggcttttgccacgttggccaggccggtctcaaactcctaacctcaagtgatcctctcacctcagcctcccaaagttctgggattacaagcaattatttacatatttggatttgttctcaccaatcttttatttttatttgtccaatttctccctatgtggccttttctctttccttgcttttttggaaggcagttctcctattcattttcttattattgcattttttccatgtgtagaaatttataaatgctatttctattattttggtgaatatctttatttattttgtttcttaaagacaggttctcactctgttgcccaggatggagtgcagatgttgtggagtgcagaatcatggctcactgcatccttgacctcctggactcaagtgatcctctttcctcagcattttgagtatccatgactacaggcacaagccaccatacccagctaattgttttattttttgtacagacaggatcttcctatgttgcccaggctggtctcaaactcctgggtgcaaacaatcctcctgcctttgcctcccaaagcgctgggattataggcatgagccaccgtgctcggccaaagacctttaaaagtataggccaggcgcataaaagtttaggccaggcacagtggctcatgcctgtaatcccaacactttggaagaccaaggcagaaggattgcttgaggccaggagtttgagaccagcctgggtaacatagcgagactctgtctctacaaaaaattttaaaaattagctgggtgtggtggcatgcacctgtagtccctgctacttgggaggctgagacatgaggatcacttgagcataggaggtcaaggctgcagtgaattgtgatcacgtcactgtcctctaccctgggtgacaaagggagaccctgtctctaaaaaaaaaaaataagtttaactctaaaaaatctagagttaaacaatatcttaattcccattttaatcaaggacctcaaaatacttcaactctctcatcctcctgccaaattatattctgccttttttatcccacagtttttatgacttttttgtatacaatgttggtttagatttaccaaaatatttaccattttatttgttcacgattctttgttgcacttcagaccattcttatgacatcagatacctccatcctaaactacgccttcagaaattgccttggtgaaaatctgattttaaaatatcggtttttgttcatccaaaaatatctatattaagatatttttattatgatatttattaagatatcttaatataataaattatattaagatataatttattaataaaatattaatattaatattttattagtatcttaataaatatctttattaagatattttttgtacgagtgcacgattctaggttaacagttacaactgacccttgaataacattgtggataggactgctgacccccaatgtagttgaaaatctgtgtatacaggaggctgagggaggagaatggcgtgaacccaggaggcagagcttgcagtgagccaagatcacgccactgcactccagcctgggcgacagtgagactctgtctcaaaaaaaaaaaaaaaatagtaaatcttgtataactttgactctccaaaacttaactactaatagactactgttgatgggaggcttaccagtaacataaacagttgattaacagatattttgtatgttacatgtattatatactgtggtcttacaacaaagtaagctaaagaaaagaaaatcataaggaagagaaaatatatttactgttcattaagtggaagtggatcatcataagggtcttcagcctcgtcttcatgttgagtaggctgaggaggaaggggggttggtcttgctgtctcaggggtggcagaggaggaaggaaatcagagtagaagtggactcacgcagttcaaacctgtgctgttcaagggtcaactattttcccgaaatactataaagagatgacacaactgttttttggatgtcactgttgttgacaagtctgcttccaatcgaattgttatttcctcctagataatccgactttcctttctgaatgcctttaagatcctctctttgtctttgatgttcttcactttgaccactgtgtatgtgcatgtgtgtgtatgtttgtgtgcacatgaagaaggctaattctgggagatgtaccttatttctcatgagttcagcaatactttaaaaagtatattttatccatgatccagttgtttcttaagaggaaggtccttcagagtacctggtctgccacaattccagaagcagaagtctatgatgctttgtacatgcacaatcctgcgtgtgcacagtggcgtggtggaaacctctgggtggacacacccctccatgcctacatctgtgtgtgtgggagcctttagtgtgcccatccctctgctatttttctagttcaaatgcaaacccttcaggtcaggggattcgctttctgtcagtctcattccctcctgacaaccttacagtacttactccccacggtcagagtaagcactggctgctgtttggtttttacacctgttgggaaaaggagtatattagtcagggttctccagagggacagaataatagggtgtgtgtgtgtgagagagagtttattagggagaactggctcacacgatcacaaggcaaagtcccacgatctgcaagctggggaagagagaagctggcagtggctcagtctgaatttgaaagcctcaaaccaaggaagccaacaatgcagccttcagtctgtggccaaaggcccaagagcccctagcaagccactggagcaagtcccagagtcgaaaagccaatgaacctggagtctagtgtctaagggcaggaggagcggaaggaagcatccagcattggagaaagaaggcagccagaagccccagtgagcaaggtgatcccacctcttccacctgtttttgttgtagccttgctggcagccgactggatggtgcccacccatactgagggtgggtcttcctctcccagtccactgactcaaatgccaacctcctctggcaacaccctcacagatacacccagaaaaaatacttcgcatctttcagtccaaacaagctgacacctaatattaaccatcacaaggagaaatgctgggagagccatggctggcccagcccctcctcactgcatcctctgctttcccatgtgtcttgtccacagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggtgagtgtcatagctgtggggtggcagtgtggatgggcttcagcagggcagccacacacactctgtcctgccgtgatccacagcaccagcctggcagagcctcttgccctgtggaagagctcgtgatggcttcttccagccatagtgtccttggggacaataatatacggctaccaggggttcatggaggtcagggtttagattaaactctattcagcaaatctttagtcagtacctaacacatgccagacactgctagacactgccaggcactgggaatccagaaatgaataagacagctgagggtgcagccctcatggggcttacaacctatgaatggtagcatcagagaatctactggtaaacaaacaattgcaaatgatactgagttctatggaggccacaaactggtagccccaagggatgctggtccatgctcctggatcggtttcctgtgtctggtatagaaaatcaccatggtggcctggccgggcacagtggctcacacctgtaatcccagcactttgggaggccgaggcgggtggatcacctgaggtcaggagtttgagaccagcctggccaacacggtgaaaccccatctctattaaaaatacaaaaattagctgggcgtagtggcgagcgcctgtaatcccagctactcaggaggctgaggcaggagaatctcttgaacccgggagggggaggtttcagtgagccaagattgcgccacttcactccagcctgggtgaaagagcaaaactccgtctcaaaaaaacaaaagaaagaaaatctccatggtttgcttaaaacaacagaagtgtatcctctcactgttctggggttcagaagtctgaggtcaaggtgccagcagggccagatttttctggaggctccaggggagaataattatttgcctcctccagcttctgggggctgtcagcagtctgatctgtggctgtctcactccagtctgcttccatggtcgcactgcctctgccccttctctgtggaatccccctctgcctctctcataagggtacttgccatgggatgtggggcccagctggatggtccagggtgatctcctcatctcaagatccttttttatttttttgagacaagagtctcgctctgtcgcccaggctggactgcagtggtgcaatctcggctcactgcaacctccgcctcctgggttcaaacgattctcctacctcagcctcccaagtagctgggagtacaggcactcgccaccatgcccagctaatttttgtattttttttagtagagatgggatttcaccatgttggtcaggctggtctcaaactcctgacctcaagtgatccactcacctcggcctcccaaagtgctgggattacaggcatgagccaccgcatctggtctcaagatcctcaacttagatctgcaaagactttttttccaaatgaaatcacatcgacaagttccagggattaggatatggatgtatctttttggggaccaccattccacctactacaccccctttgcctgtagatgggctcaacaggaccctctggggttcccacagatctctgggccaacagccacttattcccagcagaaccagccaagcaggcttctccctgggggaacacagagcttgactatccaggcaggctggggagggggtggtcggggaaggcttcctgaaagaagtagcaagctgaagggtgacacgaactacccacctaagagcaggagagaggagagagtttgacacagaggggcaggctgtgtgcactctctggatgagaaaagggtgagacacgtgttggggaaactgaggaagttctgtatggcaagaaaggtagatggaacaggaggtgaaggagtcagtttgaagagtttggtatcagggacctcattttctgcaaacaacaggaagccaacaaaaagttttcagccagcgaataacaagacagcatttgtactttagaaggatcccaatggctgtggcctagcaagtagataggatctagggagtgggtgggaggcagggagaccagacagaaggctgacaccatggtccaggcagaggcagtgatggccgtggcgttttcctggatttccactgtctacagggcaggtccgagcacctccgcgtggcccctcaggagctcctcttcctctggtggcagctctttggctctgctttggaaactccccaccgccattttgcttggatgtgtcaacccagatgctccaccctcctctggccaatccgaggtgttctcctaggatttctgaaccttgggcacagtggattggtgttttctcatctcttcctgaagaaacctccatccactcaaagctcctgctccccagatcccccaggacccggcctttccagggtctggttcttcaggtctttccttgatcccttctgcttgccagagtcagttctgtggcttatgatcagctctctctagcgcctcttctgccccatctccagctgctgggcccctactccctttcccagatctacctcaaagcacactccttcacaaacgaccctgccaggcagagtgtctccattgctgggcccccataggatgctcttctgtagcaccagtcattcaagctttttgctttccctccattcccatcggatggggccactccttacaggctaggaccagttcttgcccaactttgtctccccagtgccctaccctgagccggcacataggagggggtccagtgggtgtttgttgaatgactaagagaagtcattagtgatctgctctcccataggttcagatttcatactacggacccaagactccaccagtcaaagctctactctacctcaccggggtgggtaagtgacaaccaggatcctagagtgccgtctcatcccctgcccacctcctaatccctgcaggatgtctctgtaggagaaactacacctggagcttactttagactgagtagagaaaaagcatggtttctagccagtcagacatctgggagttcgaaatattccatccttagtcagtgaaccacaaagaaatacaatgtggcagtggtggtgcggggaattttaattatatcttaacattttgcttctcaagttaggaagtaggaacttggattttcaatatatgcatctccattaatttttttttttttgagatggagtctcaccctgtcacccaggctggagtgcaatggcatgatctcggctcactgcaacctgcgtctcccaggttcaagcgatcctcctgcttcagcgtccctagtagctgggactacaggcgcttgccaccacgcctggctaatttttgtatttttagtagagatggagtttcaccatgttggccaggctggtctcgaacttctgacctcaggtgatctgcctgcctcaggctcccaaagtgctcggattactggcatgaaccactgcgccaggccctctattcctttttgatacctcaaatatctataacaaaaagtagataatgacactgtcttatacaagaatatagatagtgttcctcaaattccattatttatatctcatcttcaccatttctgccatatcaatgatccacctatactcatatttatgtaatgttcttctttaaataaaatccattcttaaacttgacctcattttgaacaataatatcatgattagtatcattatgtgtattaaacatataagtagcagccagacatggtggctcacacctgcaatcccagcgctttggaaggcggaggcaggaggatcgcttgagcccaggagtttgagaccagcctgggcaacatagtgagatcctgtctctacaaaaaataaaaaaaaacagctgagcatggtggcatgtgcctgtagtcccagctactcaagaggctgatgtgggaggattgctcgagcccagaaggtcaagactacatgattgtgccactgcactccagcctgggtgacagagtgagacccagtctcaaaaactaaaaaatatatatatacacacacatatatatataatatgtagcatatagaatcttcaggtttcatttcattaatgcatttcattcattaatgcatatttaagtaataatactacagcacttactcaattgccaggcactgtgctaagactttacatttaatccttacaacaatccagtgaaattggtattattattatcatcccattttacaggtgaggaaactgaggcacaagagctaaacagacttgtccaaggtcatgcaattggttagtgaaggagtcaggaagtccaggccacgaaaggacaatgcttccccggccctaactaagacgtggctatgaatatgtttagaaggtcatctgggtcagtctcatgtgggacaagggtcaccatatcatgctgtggggcacacggatctattttattggaatgaccttgcagtcatatattcggatgagggaggaaggatttacaaacaaatgtttgctttctcagccaagccatttggacagagtgggggcatgttcagggttgcaatttgtccccagatacagtcacaacacatgtcatgaagagcccgtggggctggtgtcaccgctgggtcccccaccccaaggtcactgagggctggggccagagtctttcgagaggctggcagggtgtgcctgcagaggcttcacatcgaacctctcattgtggtggttgggaattccttccaaaatccctgggggtggccagacgtgcagggtctggtggacatcacagttgtggccccgctgaaggggaacaggtgtggtcagaggccccagctctgctgtccgaactgtagccagacttcctgggggccgtggaggagggatgtcttgaacctgtgtctcctctgaaggacgggaagaggggctcacactatgggtgcacacagtcccctggggaaacgacctgcccattcagggccaggctgggtgccccaaccccggaccctcaccaacctctcctcttacttgatgggatttcagaaatctccttgtgcgcagacatcacccgcaccggcaaagtgaagccaaccagagctgtgaaagatcaggtaccactcacccaaacgctcctttcctacttctactggattctccccgggcgcccccttgtggtgatggggcaaatgctggcctgtcccacgtattccgtgttaactgttaaaaaaagaaagaaaattagcatcaccattaatattttggcttatgtatttcccatctattttacaaaatcagaatttcaccaattgtgggttcagatgtgttatcctacaaactggaaaccctactgagtcacccagttaatacacagcagagaggaaattcgtgggcaggtctcgactctggctcaaaaactccctactccatgctctacagctgatatgggagagatttctgcagagctgtctacatgggggaataacagagaaaggaagaaatggcagggggagaggcgggaaggagccaggtggggagagggagggtgtgttgtggaaagacaggatgatgggatgtgcagaaagggcaggtgaacgctgtcttaaaccagagtctgcaggtaaacctgaaaccccagagctgcctgcaccattttatgctggtgtgcactgggggctgtgccctgtaaaaagatgaagatcctttgtggggtcagcaaaagtgggaagacaaattacaactctaggagtagaatctgtccctgtatccctcccgaaatgcagtcatttcccaggggcaaacgctctcccgcgaagggaggcgccatcactattagatccctacagggtggggctgcttaatgtgctttaatctctaggcagatattaactggctgctaagaatagtaccacactttgctgaatgagtaattaagagttcacataatagtaactatttagattagaatattgacatacatatttagctgcatttggtttgtcaagctcaacattttatttatttatttatgtatttatgtatttatttatttatttattgagatggagtcatgctctgtctcccaggctggagtgcaggggcggttcaccgcaacctccgcctcccaggttcaagtgattctcctacctcagcctcctgagtagctgcaattacaggtgcatgcctccacgcccagctaatttttgtatttttagtagagacagcgtttcgccatgttggccaggctggtcttgaactcctgacctcaggtgatctgcctgcctcagcctcccaaagtgctgggattacaggtgtgagccactgcgcccagccaacatttcttaaaagctgttaaaatccatgtgactgtcttgggctcctttttttaaatggatgaattgtgttttgggtgaatttggttgtgacttttggagtaaaaggcaaaaatccactcctgagatcaaataattaattgatgtggaagcaagatgtgtaaactgcactgggttaaagaagtgatgggggccgggcgtggtggctcatgcctgtaatcccagcactttgggaggccgaggaggatggatcatgaggtcaagagattgagccatcctggccaacatggtgaaatcccatctctactaaaaatacaaaaattagccaggtgtggtggcatgcgcctatagtaccagctactcaggaggctgaggcaggagaatcacttcaacctggggggtggaggttgcagtgagccaagatcacgccactgcactccagcctggcagcagagggagactccgtctcaaaaaaataataaataaataaataaattttaaaaaagaggtgatggggagttgctatgcctcttgcaagagttggagggctctatgctagtttctgagcatgactcttccctgctggtgtccagaggacctggacctggggcccttgtggacagggtgccatcctgctggtgaactgtgacagagacaatctcgaatcttctgccatggactgcgaggatgatgaagtgcttgacagcgaaggtaaagagcatttgctagctaacgggaagggctttttataaagcccttttgcccacatagcctgaacctggtgacggccctctgaaataagggttattgaaagcatcttcttgttggcaaatctgttttataggtgaagcaatgggcaaaggccctcgggggtcaagtgacctccccaagctcacgcagcggtcagggaagagctgaggccagtgctgtctcctgatgcctcctacaggccctggtttccatgcctcaccctgctgcccaacctaaacgctcttaggtcaaacctggagcctgaaatccttcgaaatgaaagcttttcattcccaaactcatatcagagcaggccctgagacctctgccataggccgggcacgactctgaccactttggaatgattaactcatctgatcttccaacacaaacctttataggtaggaaaaccaaggcacggggaacttaagcaatttgcccaaggtcacagcactgacacgtagcagactcggggtttgaaccccggacgtcccaacaccggagcaggtttagaggcatgaggatggtggggaatgcaggatctctgcaggcaaacctctgggctgctaagggggtggctccggggttgaagtgaggaaacattatccacctgcgctggaggaaggtgctaaggggctctggctggcaggccttcctggggagctcttagaataacatagaaatggatgtattttcaagttgcatgtagttggcctggcccgatgccaggggacagcctctgtacagtgcctgacatgcggtaataatcacaacagctgtcctttgtgggcactcccggggtgccaagagctggtataagcactgtgtatcacctgactttatcttcacaaccattctacggtgggtacaactcttgcccctgtcttagtgatgaggaaacagaggcagagaaagctgaaatccgtttcctaagcccacccagatcccagattcaaaccaaattgaccgcaaagcctttaagcccttggctatcagtcagtcccttgggaaccatctcttcactctccccaccctctaccccttcttggctattgttcaagttggttcacaaaccccacttcttgctcaggaccagcacagaaacctttcactcgttcattgcacaaatctcagccgagcgcctgccgcctgctggtcattgttctagggagttctcggctttctaggttcaagattcagggaacgttgcaaggagcaaaatattaataaatgtggtccccgctttgctgaggttacatccccagctcatcctagtgggaggagaccaaaaccttaatgagctctcattccaaaatgaaaaccatcatttaaatactcaaaatagcataaagaaaagaaaaccaggcaacatggtaaagggaggtctcaggggagcggtggggtgtgaggctccaccaggaggtgaggtggctatgggaaaccagcagcggtctcagggccctgtgactcaggcaatgcccttctcatcccagacctgcaggacatgtcgctgatgaccctgagcacgaagacccccaaggacttcttcacaaaccatacactggtgctccacgtggccaggtctgagatggacaaagtgagggtgtttcaggccacacgtaagtcatccccatctttgttcccctcctgccctggccaacggggcacaatcctgtcacacagctgtgtggcagataaatcctgagcacctactatgtgccaagttctaagaacagcagacgcagcagtgggcaagacagacgacatctcgtctactaccttccagttggaggacacagacaaaatgactcgttattgctccctgcttttctttctctctctctcacacacgcgcgcgcacacacacacacacacacacacacacctcctggttatagactttgagtgcaagctgacagcatgtggcatgtaatcatttgttctgcaacatcctgctaatcagagacatcaggaacttctcagtgacccacgagcacctgcaactaagtaggtgccaggttgatcccaggcacggggcacagagccatgactgtgaccgagtccctgccctcgtgttgctttcatcttagtggctgggagaactataggtaaaataattacataaaacacctagtgtgtccaacgctgtgacatgctatgtagaagaatgcggcagggaaggggatgtggagtgaggagtggagttcgggttgtgtggagagagggtggccagggaaggtgtcccccatgaggtgccatgtgagcagagggaggtataggaacagccagacagacacctaggggaagggcgttccaggcagagggaacggcaagtgcgtgggccctgaggtgagatcatgcctgtggtttggaggaatgcagggaggctagtgcagctggggcggaatgtgcaagggttaaagtgggggatgagggtggaagagacagggcagaggggaagggggaggtcaggtaggcgtatattgactttggcttctggatggaaggagatggagagcgccatgggagggttttttttttgcatgggcctgacaatcattctgatcctgatcattctgcctggtctattccaagctttttttttttttttttaatttttttttttgagagagtctcactctgttgccccggctggagtgcagtggtgccatcttggctcactgcagcctccacctcctgggttccagagattctcctgtctcagcctccccagtagctgaggttacaagcatgtgccaccacatccagctaatttttgtatttttagtagagatgggatttcaccaggttggccagacaggtctcgaactcctgacctcaaatgatccacttgcctcagcctccccaagcactaggattacagacaccatgcccagcctctgtttcttttttttttttttttgagatggagtctctctctgtcgccaggctggagtgcagtggcgcaatctcaactcactgcaacctccacctcctgggttcaagcaattctcctgcctcagcctcccgagtagctgtgattataggcatccaccacaatgcctggctaatttttttgtgtttttagtagagatgggctttcaccatgttggccaggctggtctggatctcctgaccttgtgatcctcctgcctcggcctcccaaagtgctgggattacaggcgtaagccaccacacccggcctgtttcattcttaaagtgattaaactgacactgatttggtgacaggtcaacttattgcctccacccaggcatcacctcctccaggaagttctcagggattccctctatcacagctgtctgctattttcatgatctctgcttctccctactcacccgtaagactgtgcactctctgggggtgaggataggaggacagtgactctgtctccctggtcccctgctgtgcccctgtcccccgctgacatctggctagagcagatgctcaggaatgagtgaaggaatggtgtatactggacttcctataaagtttggaatagctcaggcacccctgccacacccttctgggacttcagccggggagcagcgtcccagctagaacttcagtccttttctcctaaggggactggccagatttggctgcaaggggttctctgatggtgccatgtggtgaccctgggaaaggcttctggggagcactaaggagaggaggaaaagtcttcagaagtggggacgcagacctgagtctcccctgcctctctcctagggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggtaggccgagaaggcagccctgcatcgggggcctgggcttccaggcagtggcctggctagggaaaggggtacagagcccagctgggcagggggactccaaacagctgcaggaagtggtaggtgctgggccctggcagcggtgacagcccctccttccccttaccccctcccctgcaggagctccccgaggctgtggtgttccaagacagcgtggtcttccgcgtggcgccctggatcatgacccccaacacccagcccccgcaggaggtgtacgcgtgcaggtgagaggtcctggggtgctggggtgggtccagacaacaaagagcctgagttccatccgcagcactcactgtgtgatgggaaaaacagtcacccctcccctgcccactgagagcttgctgcttgttgggggctctaaaaaggcagggaagttccctactgttgttgtcattgcctctgtcctgccctgatgcagctgaggctacgttaactcccaggggcctgtagagtgtgtcccatggaccttgtgcaccagaaactcctgggaactcatttaaaatgcacctttctgggctgggcacggtggctcacgcctgtaatcctagcactttgggaggctgaggcaggtggatcacttaaggtcagaagtttgagatcagcccagccaacatggtgaaaccctgtctctactaaaaaatacaaaaattggccgggcgtggtggcgcatgcctgtaatccagctactcaggaggccgaggcacgagaattgcttgaaccctggaggcggaggttgcagtgagctgagattgcgccactgcgctccagcctgggcaacacagtgagactccatctcaaaaataaaataaccagcctggccaacatggtgaaaccccgtctctactaaaaatacaaaaaattagccaggtgttgtggcatgtgcctgtaatcccagctactcgggaggctgaggcaggagaatcacttgaacccaggaggccaaggttgcagtgagccgagattgcaccattgcactctagcctgggcgacagagcaagactccgtcttgagaaaattaaaaataataaaaattaaaaattaaattaaattaaattaaatttaaattaaataaaatgcacctttctggctgcctgtgagctgctgggtcttatccctgaggacggggcccatctgcattttggggaggcccaggcaatcttcctgcagtctgagagccgctgacaagtgtgctggggtctcaggagaccctctcctcccctcttctgatagcttcaggggcagaagagtaagagggtaatgtggtcaggacgcaggctggaatccagtgaggactgtattccggtcccagcactgttccaggtggtgtgaccttgaacagtgaactcatattctgtcttctcccaagttgatatggtttggctctgtgtctccacccaaatcttatcttgaattgtactcccacaattcccacgtgttctgggagggacctggtgggagataatttgaatcatggaggtggtttgccccataccattctcatggtagtgaataagtctcatgagatctgatggttttatcaggggtttccacttttgcatcttgctcattttctcttgccgccgccatgtaagaagtgccttttgcctcccaccatggtgtccccagccatgtgggagtgtaagtccaattaaacctcttttccttcctagtctcaggtatgtttttatcagcagcgtgaaaatggactaatacagtaaattgataccagtagaggggggcgttgctgaaaagatacccaaaatgtggaagcaactttggaactgggtaacagacagaggttggaacggtttggagggctcagaggaagacaggaaaatgtgggaaagtttggaactttctagagacttgttgaatggctttgacaaaaatactgataatgatatgaacaatgaaatccagactgagatggtctcagatggagatgaggaacttgttgggaaccggagcaaaggtgactcttgttatgttttagcaaagagactggtggcattttgtacctgccctagagatttgtggaactttgaacttgagagagatgatttagggtatctggcagaagaaatttctaagcagcaaggcattcaagaggtgacttgggtgctgttaaaggcatccagttttaaaagggaagcagagcataaaagttcagaaaagttgcagcctgacaatgcaatcgaaaagaaaatctcattttctgaggagaaattcaagccagctgcagaaatttgcataagtaacaaggagattaatgttaagccacaaggcaatgaggaaaatgtctccagggcatgtcagaggtcttcatggcagcccctcccgtcacaggcccagaggcctaggagaaaatggtttcgtaggccaggcccagggtccccatgctatgtgcagcctagggacttggtgccctgcatcccagctgctccagccatggctaaaaggggccagtgtagagctcaggctgtggcttcagagggtggaagccccaagccttggcagcttccacatggtgttgagcctgcaagtgcacagaagtcaagaattggggtttgggaacctctgccttgatttccaaagatgtgtggaaacgcctagatgcccaggcagaagtttgctgcaggggtggtgttctcatggaaaacctctgctacagcagtgcagaggggaaatgttggtttggagcccccacacagagtccctactgggacaccacgtagtggagttgtgagaagagggccactgtcctccagactccagaagggtagatccactgacagcttgcaccgtgtgcctggaaaagccacagacactcaacaccagcctgtgaaactggcctaccaggagggaggctataccctgcaaagccacaggggcagagctgcctaagaccatgggaacccacctcttgtgtcagcatgacctggatgtgagacctggagtcaaagatcattttggagctttaaaatttgactgccctgctggattttggacttgcatgggccctgtaacccccttgttttggccaatttctcccatttggaatggttgtatttacccaacacctgtacccccattgtatctaggaagtaactagcttgcttttgattttacaggctcataggaggaagggacttgccctgtctcagatgagattttggattgtgaacttttgggttaatgctgaaatgagttaagactttgggggactgttgggaaggcatgattggttttgaaatgtgaggatataggatttggaggggccaggggcaggatgatatgctttggttctgtgtccccacccaaatctcatcttgacatgtactcccataattcccaagtgttgtgggagggacccagtgggagataatttgaatcatggtggcagtttctcccatactgttctcatgatagtgaataagtctcatgagatctgatggttttatcaggtttttctcctttttcttcttcctcattttctcttgccaccgccatgtaagaagtgccttttgcctcccaccatggcctccccagccatgtggaactgtaagtccaattaaacctctttttcttcccagtctcaagtatgtctttatcagcagcgtgaaaacggactaatacacgggtggaaggggtacccactagttcctgcctcataacttgttagatggattccatgagctaatgcctgctaagagcagatggaccggacgtgccaggagcctctgttcaggccacaggtgacccctgagccacctgtgtgtccctcccaatccttccaggctgagcttcaaattccagagcactaaggagctgcttttctgctctctctagtatttttgaaaatgaggacttcctgaagtcagtgactactctggccatgaaagccaagtgcaagctgaccatctgccctgaggaggagaacatggatgaccagtggatgcaggtatgtgccctgcggggcaggcagggtgactgtccctgagggccaagagacacttgggggacccgggctcctggggttcagcctggtgcctcaccggccactcttccttagatggacagggagataggaacctgagagatccttgggccgggaggctcaagcaagtgattcatctgacgtttgctgtgggccactgcaggcccactgcagtcaccaagatgaagccaccttcctgcttgaaaacactcaggtggcctcaggaaatgatgtgaagctcaggagattggtgctcaaccagcccctcaagtggtcctccctccaggctgtccatcttgggcccagccaccaaagcaggtcacaccccagctccagcactttccatggctcccatctcctcctgcttagtctgtttccaggccctctcagcctgcctggttcccttccctggggccagcctgccactgtgcctggaaacacatgattaccagtctctgtttccgtgccactcccccatcatgccaggagtgccccaaggactgagactgggcctcatccagcctagcaccctgaccctccccaccactgccttgctttgcctcgggaccctgtccttccatgccgcacccctgcggtgctgtctcttggacccatctccccattcccatcccccatctccaatccataccggtgaatcccagcacaaatgctcctcgtccataaatttcccccatcacccccagcaggaattcatccctctttcttttcacccccacacaacatcctttcttcctttcttgccattccatcttttcttttcttttttttttcactcttgttgcccaggctggagtgcaatggctcgatctcggctcatcacaacctctgcctcccgggttcaaatgattctcctgcctcagcctcctgagtagctgtaattacaggcatgtgccaccacgcctggctaattttgtatttttagtagagatggggtttctccgtgttggtcaggctggtctcgaactcctgacctcaggtgatccacccacctcagcctcccaaagtgctgggcttacaggcatgagttactgcacctggccgccattccatttttttgataccaagttgacctgtgtgaaattgctgatatgtgccatttttgatttgcaagagtggaactttcatctggttcatcctaatagtaacttatagtagtgattatgtccaacatcacacctgcctgctagaatacaagcctgcggaggcggaattttcagcagattcatttctgaaacctccccgtgctcctcctgctgctccagtggcccaggctgggtcttatctgccctcctttggggaggaggccgcgagctctggcgccaacaggtcttctctggcccatcctagctgcatcaggagcccctcctctgtgtccccacagacccctcaacccctcaccccccatcacctcctaaccctggtgcccactgcctggctcccttgttttctcaccttgaggaggggtctcagttttgctcaccaggggtccccagcactggcccaggcaccaccaggagtaggagggaagggcactcctgcaggctcatggcctctcgtgcactgggcaaacagggggcaataagctccgcttctgagccccttctctgagatcaaacatcccataggaatgtgcccttgactgcaagggtgagagtgagtggatggtttcatgcccccatcctggcgtcgggcaccaagacccaggcagcacgcgcaacagcctcctctccactcactcccacaggatgaaatggagatcggctacatccaagccccacacaaaacgctgcccgtggtcttcgactctccaaggaacagaggcctgaaggagtttcccatcaaacgcgtgatggtacctgcatggggtggggagggggcacagctgccgaaaccctcttgtcttgagactccctccttttagcctgccttccccgcccgcgcctgtagctgagtagcccagtgcaaggaggtggaattcctaccaaagtgggagcatgtaggtggggcttgttatttttttattaccaagataagatctggctcttcatttcccaaaacccttgtcacccgtgagctcagcttgatctccaggtaaccctgccgaggaggtggctcaggcttggacctccctgtgctcttgaagaagctcttgctgcaaggtggaggtggggccggggctggaatctttcccaatgcccagctggaggctctgtcttatgggtggtgtttggtcttcaggggctccatctgggatatgggggattttgggatcagtggatcagtggtcatttcctgggtgtgactttatcagccacaggctgtccatgtggaggtgggaagtgacgtattgctgactcatgcttgactagcctggcaggttgatgcaaactcagcacagaacaataataataatagttgcatttattgagaatgagttgtgtgttagccagtgctgcattctttacctgcactgactcatttaatttgataaactgttatggagattctatcaataggcccattttatagaagaagaaactgaggctcagagtggagaagttggaattcaaacccggccagtccggcttggtgagcatgcttttccccgccctgcctccctctccctgctttcccaggaagaccccaggcctgcgctctgagtcctggcagccccttggctcaaggtgcccagggaaaacgacggaaggagtgtggggtccaaggtttgctaggacaaatggctttccgacaagcatctgtactgtgccccaagggaggggagagtggccctgcccacacagaaggcttcagaatgaaacttcagttaaactcaacaacttgtcccgagtctcgcttcctcctggccaagctgtgcgctgagcagtggagatgcagagatgctgttggattcacagtgtgtactcagccatggggtcgagttcatcctgtttcagcagcgttggggcgaaccagccagctgctaccgtgactttgccctctttctgctgctttcagtcctgcctggttcagtggctgtgtctactttgatgctccaagtataacaaaggccagattttctgatgccacaagtcctgctggacagacagacggacacctcggcaccattgttgtcccctttcccacagacaactttggatgaaggcaggcagcactggtccctcagctaagcacatctgtgtattccaactcaaatcactcagaatgaggccaggtgtggtggctgacacctgtaatcccagcagtttgggaggccaaggaggatggatcacctgaggtcagaagttcgagaccagcctggccagcatggcgaaatcccgtctctactaaaaatacaaaaattagctgggcatggtggtgtgtgcctgtaatcctatcttctcaagagtttgaggcaggagaatcccttgaacccaggaggcagaggttgcagtgagccgagattgcaccactgcactccagcctgggtgacagagcaagactgtatcaaaaaaaaaaaaaaagaaagaaaaagaaaaaacaacaacaaaaaacacccaaatcactcagaatgaagagaattggagaaaaagtcacactcagcaccaggctttggccccacctgcctctccagcaccattgtaggccactctcgctgtcccttacccagccccacctccactggcctccttcctgttcttcaaacttgcctaacgtttccccacctcacggtctttgcatatgccgttccctctgcttgcagtgctgttccctgttcacggggctggctgcttctcagccttcaggtttcagcttctatttcctactcagtgaggccttccctgatgctaaaagagggatccctactcccaccccaactcctagccttctccactacagccccctgttgacttcttcagtgtattttgttaaaatcatggactcgttcatctacttttcgttgcctgcgctgctgggcctgaggctgttatgccttacgctgtccatcgggcagctggcacaaagcttcacacacagtggttgttcttcctgtaagcatcgtcagtaaattttctatcaccaccatcaatgggaaaccagcagcacttcctgcagatagaaggcaactgtgaagttaaacagaatgaaacaagatcctgctaccaaagccagctcgttacaagtgactgcagcatctctgagctcgaggtcagctctctgctggttaaaatgagggattagcaggtgtcaaggatgtgtttgcatcactgaaacttgaatttttgaggaaccagaaaagactggaggaaaatgagaagggcaacagctttccaacgttgtgcctcaggccgcatcacctggcatccatgagcgtcccccacacagcccactcttggggggccacttctgcagactatggatatgccttcatttgcctgcagaaatccaaggctgaaggctgtataactgaccccctctcacaacaacagccagggcagtgatgtggctgaatttacattcattcatcaaacactgtttgaacacatactcagcggggccttgttctaggcacttgagatatgacagggagcaacgccaaggtccctgacctcacggaggggacgttctggcccagagagacagacagcagacaatgaatagaacaaagcagttatatggttccttagaaggggatgtcaagagctacaggtgccaggtgtggtggctcatacctgtaatcctagcactttggaaggccaaggtgggagaattgtttgaacccaggagtttgagaccagactgggcaacataatgagaccttgtccctacaaaaaaaaaaaaaaaattgtttcaattaactaggcatggtaacacatgcctgtagtcccaactactagagaggctgaggtgggaggatcgcttgagcccaggaggtcaaggctgcagcgagtcatgattgcacttctgcactccagtctgggtgacagatgagaccttgtcccagaggaaaaaaaaaaaaaaaaaaagagctacagggaaagacagagtagagcggaatggaggggctcataggtgccagcgtggtagggaaggtggattaggttacattcttaaagccgagtctggtggctcattccagtaatcccagcacttcgggaggccaaggctggtggattacttgagcccaggagttcgagaccagcctaggcaccatagtcaaaccctgtctctacaaaaaaaaaaaaaattaaaaataaaaataaaaataagccaggcatggtggtgcatgcctatagttccagctacttaggaggctgaggtggcaggacccttgaaccctgaagggtgaggctgcagtgagccatgatcacaccactgcattctggcatgggcaataggagtaagacctggtctcaaaaaaaaaaaaaaaaaaaaaaagattgcgttcttaaataggatggtcagggtgggcttcacggagaagctgaccttgcagcagatgcttgagggaggtgagcgagttggccatgtgagtgggtgtctgggtgaagagtgttgtaggcagtgaaacagccagtgcaaaggccctggggcaggagcatgccttgagtgttccagaacagcaagagggcctggaggggtagagcagagcaaccaggaggagaggcagtcagagaggagataggaggtggtagaaaaggcagtcagggagctgatgggaggcagtaggagaggcggtcagggaggtgatgggaggtgggaggagaggcggccagggaggtgatgggaggagaggcagtcagggaggtgatgggaggagaggcggccagggaggtgatgggaggagaggcagtcagggaggtgatgggaggtgggaagagaggcagtcagggaggtgatgggaggtggtaggagagactgtggtcagagaggtgatgggaggcagtaggagaggcaatcagggaggtgatgagaggtggtaggagaggcggtcagggaggagatgggaggtggtaagaggggtggtcagggaagagatgggaggtggtagcagaggtggtcaaggaggtgatgggaggagatggggggcagtagaagaggctgtggtcagaggtgatggggaagggacgggatgaccggatggggttgggcttgttggctactgttaaggctgcttgtttagaaactgagaatcatcacagggtcttaaacagaggaggattatgatctggtttgttagaaaagggaacaagccctgctatgtgttgttgagtaaagagaacagaggcaaggggcaagggagtgggcaggtgagggatgctgggagtgcaggcagggtgtggccagaggaggtgagaagccatgagattccggctatgtctggaaggcagagccagcagggcattctgacccccgctatgagagaggcaagccaaagctgacagcaaggctgtgacctctgtgggtggcaggcaccatcccctgggaggggaggactgtgggaggaggaacagattgatggagaatgagggggtgggaagaggggacccaaagttctgtttagggcaagtgggagacaattggtcaacatccaaatgggaagtcaagtgagcgtggatacacatgtgatgtttgggagggaagggatgcccagagatgcgaacatgagggttgtcggctcgggaacggaattccatgagacttggcaggacgactgaaggggcaagtgaggttggaggtgaaccaggatcccccggtgtcctgaaagccaagtgcagaaagtgtgcggagaacacaggtgtgaccgcctgattcaggggcttctgaggggaaggcgaggcccgcgcattgcacttgggatatggccatgtgttcatggagacctggacaaggaaagctttgatgggcgggagaagcgggttggtctgggttttagagagaagtggagagaggaactggaagtgtgattgttaaatctgtggaatcaggccaggcgcagtagctcacacctataatcccaacactctaggagccgaggcaggaagattgagcccaggagtttgagaccagcctgggcaacatagtgagaccccatctctacaggaaagaacaactttaaaaattagccaggttgtgatagcatgcacctgtggtcccagctactcgggcggctgagacgggaccgtgatcctttgagcccaggaggtcaaaactgcagtgaagaagtaaatttgaagatcacagacctccttaagacccactcatcttcccagaaactcacacctgggcacataatttggcgtctactctcagggagttcatggcccccatgaagcctcagggaatggaccatctatgtgtagaaggctagatgtaccctctcctgggaggcaggggagatgggcggtggactgaataatggcccctctacatcctagtccccccgaacctgatgtttccttatatggtaaaagggactttgctgaatgtaattatattagggatcttgtgatggggtgattatcctggtttatcctggaatgcgggagggcctgtgatgatggaagcagagactggagatctttgaaggtggaagaaggggctgccagccaaggcggctattagaagctgaaaaagacacggaaatataatagattctttcctcagggcctccaggaggaaccagctctgccatccctggacttaagcccagtggaactgatttcacacttctggcctccagaattttaagagaataaatgtggctgggcgtgggggctcatgcctgtaatcccagcactttgggaggtcgaggcagatggatcacttgaggtcaggaatccgagactagactggccaacatggtgaaaccccatctctactaaaaaaaaaaaaaaatacaaaaattagctgggcgtggtggggcgtgcctgtagtcccagctactcaggaggctgaggagtattccttgaaccaggggcggaggttgcagtggctgagatcatgccacggccctccaacctgggcaacagagtgggactctgtcttaaggggaaaaaaaatgatgttttacagatttgttttatgcccaagttaaggtttaccaagcagctggcacgctttagggacccaacaaatgctagtcccttttcttgtccattgaatgaactcaacattcctttaaccttccacatgtgataacagcacctgctatgtgccagctgtagacggtactgagttacttcctgtgcccaagttcatctctaaacttggaccccccgacccttcaccagggacctcattcctctaactcttggcactcccttctcctatctcagggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccaggtaaggaggggagtaacaggaaggggtggccaggacccaggtatgccgtagaaaagcagaggccagagtggaagccttgccttcctgcttccgattctagacagcccaacagacttgagggagtgtgagaggaatccaagcgcagggtgaggaatgcgggctttggagcccatagacccaagttcaattccagccctgccacttaccctctttcagtcagtgaatatttactgagcatccaaaatgttctgggagctgggaatgcagccgtggacaagaaagacaagtccttaccactactctagtagggaaacagatgatagaaaagtagacaagataacttcaaatgacaaaaacaaaagagaggggcacgggaggaataggaactgttagggtgaccagggaggtcctctctggggaaggggcttttgaactgaggcctaatgacagaagggacagggccacctggagatctgggacagagagttcctggcaaatgaaagtgcatgtgcaaaggccctgaggcaggaatgagctcctgcgtgcaaggccaggatggctagaacctattgaacaagaagaggctgggcacagtggctcacacctgtaatcccagcactttgggaggccaaggcaggtggattacctgaggtcaggagttcgagaccagcctgactaacatggcgaaaccccgtctctactaatgcaaaaattagctgggcctggtgatgcatgcctgtaatcccagctactcgggaggccgaggcaagagaatcgcttgatcccgggaggcggaggttgtagtgagccgagattgtgccagcctgggcaacagagcaagactctgtctcaaaaaaaaaaaaaaaagtgaggaagaacaaggcactgcaacactaatttttttttttaggagaaaactgaggctcaaacaggtgaaggaatgtccccaaggccacatggctaacaagagatcaccgacttaggtcagactggctctaagccccatgtgtacccagaataccagcatctcatctaactcaagaggcagtgctttctgaggctaagctctgttctacttaccttagtgactctgtgcctcagtttcccccaataaaactagaatgacaattccttgatttctcaaatgttaggggataaaataaaggcattctagactcctgcatccctttctcagctgaaggagaaccctgacctttttgccaagccccttgtccttcagggacttccctgtagcccttgctgccgataacacccctttaaccctgccatgacagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaaggtacagtcttgggggctgcctcaggaagccatgcctccttcctgggtagaccctctgcctggggtgggagcaactttacttgtctatttctccttcacccttaggatggcagtagaggaggtggccagcttgggtccaagtccacactactcccaccctcagcagatccaccctcgttgggagctccaggggcaaagctgacttctaaccccagtgtttctgcctccagggcttccggctgctcctggccagccccaggtcctgctacaaactgttccaggagcagcagaatgagggccacggggaggccctgctgttcgaagggatcaagagtaagtcggccctgccttgttctcctgtctgtgcaccttcctgcttcccatagtccgctgttgcctggagggaatcatccaggcaatagggtagcatctgagcacctactgtgcgccaggcactgtgccaagtgctagggagactgcatgaacagggcagaacagcttgctgccctgtgggctcacaggccagggggaagtgagtcaaaagaacagctccaacacaggggctttgaggggtgtctgagtggatggagctcagggaaggcttcttggaggaggcagaggccaagcagtagcatgcaagtaggggttggctgggcaaacggggcagggaaatgagaggaagagagataccacaggcagagggacgggggtggggcggctcagaggcaggagaaagcacgatgtgtgtgaggacctcggggaggttcggtatagctggagcacagatgaaatattactctctcagcagagctcacagacatgggccaggccccgggctgagatgcctctgtggctgagagctccacctcagatctgagtatgttgtgtggcatcaggagagggctgacctgtttcttcctctctcgatgggatctctttggagataagataatttagcgttaatgcaaggcaaaatgttccagtgaacaagtttcatggttcaactttataataattataagtaaacctgttaaatttttctggacattcttttcttttgaaacgaagttttcctctattgcccaggctggagtgcaatggcgcgatctcgactctctgcagcctctgcatcccgggttcaagtgattctcctgccccagccttccgagtagctgggattacaggtgtgcaccaccacacctggctaatgttttgtatttttagtagagatggggttttgccatgctggccaggctggtctcgaactcctgacctggtgatccacccacctcggcctcccaaagtgctggggttacaggcatgagccaccacgcctggcttaaaacaaggacatttcttattgacagcaactaaatggtacttgtagcatttttatcacacagtagattccatccattcactatacttttctgagctgtctgtcctgcatgcaagtagatatttttaatgttgtctgttttctgtgctgttcctgtaagtgtgctattaaaatacactaaactaggctgggcgcgatggttcatgcctgtaatcctagcactttgggaggacgaggcgggtggatcacgagttcaggagttcaaggccagctaggccaagatggtgaaaccccgtctctactaaaaatacaaaaattagccgggcgtggtggcgggtgcctgtaatcccagctacttgggaggctgaggcaggagaatcactcgaacccgggaggtggaggttgcagtgagccaagaatgtgccactgcactctagcctgggtgacagagcaagactcagtctcaaaaaaaaaaaaaaaaacaagaaaatattaaactaggccaagtgtggtggctcacacctgtaatcctagcactttgggaggctgaggcgggtggatcacctgaggtcaagagtttgagaccagcctggccaacgtgatgaaaccccgactttactaaaagtacaaaaattagctgggcgcagtggtgcgcacctgtaatcccagctactggggaggctgaggcaggagaatcgcttgaactcgggaggtggaggttgcagtgagtcaagatcgcactactgcactccagcctgggcgacagagcaaggctctgtctcaaaacaaaaaaaaaattacactataaaaatatattttaggccgggcgcggtggctcacgcctgtaatcccagcactttgggaggccgaggcgggcggatcacgaggtcaggagatcgagaccatcctgggtaacacggtgaaaccccgtctctactaaaaatacaaaaaattagccgggcgaggtggcgggcgcctgtagtcccagctactccggaggctgaggcaggagaatggcgtgaactccagggggcggagcctgcagtgagccgagattgcgccactgcactccagcctggacgacagcgagactccgtctcaaaaaaaaaaaaaaataataataaaaataaaaatatattttaaaaagaatttaaatgaggtgagaacaacatttttaatgagaacttatactgctaaaatattgtattaaagatatttgacttgcagatgctcacagcaaacctgtatggtaagaattattactgactccattttatagatgggaacactgagtccccccccggcactggctgggaagagggaaatcgacctctaagttcatttgccttttttttctttttctccatgacagaaaaaaaacagcagaaaataaagaacattctgtcaaacaagacattgagagaacataattcatttgtggaggtaggagcctgggtgcctacaccccagcagacctgacgccctgtccccggctcagccactttcccagtgattagaggcacacagaggctcagggtctcaggatgcgctggaagacagagacacagaagcaagggcagaagcaaagactgggagaggctgagggagcagagggaatgggaggccccagggtcccccgagagcactggccagaggcccctctgtgcagtgaggcctggcagccaccttcactgccttcctgacactgtcccaggtcctaccctccggcagggggcctcagccccacactgtcccccacccccacccccgactgccatcagtcccccactcactgcccctgccccttccccaagagatgcatcgactggaaccgcgagctgctgaagcgggagctgggcctggccgagagtgacatcattgacatcccgcagctcttcaagctcaaagagttctctaaggcggaagcttttttccccaacatggtgaggaggtggcggctttaaaaccccagggtgtggcatggaggtagctcagcccgagaggccagtggggcacccgggcggtcccagagggcttggctcctctctgtaaccgtttgtagctttgtcctgagtggtacaaggtcagacgtgaccaggtccatgcacgttggtgtctttccacaaggtcaggcttctactgatgctatttccatcataaaatccacaagccacacggagttccccaggggcagtcctcaggtgggcagagccctgggcacacaggctcaagccactccacaagtcagtcagtcctgcaaaccatacatagtagtgtacttaatcaacacagaaatgttacagatgaaacattcttaccaaacaaagcaatatttaacatcaagagagaaggggataggaaaaggggtcagtgaaccagtccaaggagagtgatgtggacaaggagagggtcctgggctgatctaaatggacatcaacgtcttgcaaggaagagtgtaattttggcagagccttcaacagcgggtgccaggtgctaatcactagtgacagcaagacagtgtctgttaagacagtcatcttgaggccgggtgcggtggctcacgcctgtaatcctagcaccttagtaggccagagcgggtggatcacttgaggtcaggagttcgacaccagcctggctaacatggtgacacccccatctctactaaaaatacgaaaattagacaggtgtcctggcaggagcctgtaatcccaactacttgagaggcggaagcaggagaatcacttgaacctgggagacggaggttgcagtgagccaagatcatgctattgcactccagcctgggtaacagagtgagactttgtctcgggaaaaaaaaaaaaaaagacagtcatcttgagctagtgaagacctgctctttttatagccagagtcctctggtgaggactgatagtaatagagtatgcctgcttatgtccctatctggttgggtgcagtctctgttgattaggcaaacatctggttcctgttagcatggtgccttttgaaatgtaagatggagtctttttctaagatggagtcacttatgccaagggtgctctatacaggcttgcatggccctgtaccacttccagagcaccttctggtatctcacaacaattccagaagtcagagtctatcgtcgcttctgccagggctctggcttcaaaccccatgccgtaactaccatgctgccttcccacgtagcaccttccctggggctccggggtgatgcaggggatggagggatggatagggaatcaaccaacaaacacgacaccctcaagtcaccttatgcaccataacagggacacacgtgggggcaccacctcagactggagggtccgggaaggcctctgtgaggaggtggggctggcactgcctaggctgagtgggattcaccttctggaggctgcacttgtgggtggtcccacctttgtcttgagcgttcaccgtggcccctaagtgctgtcttgatatcatccattgtgtttttgccattccttggccccattcctccctctcttcagagcatacccgagaccgaggcagagacttcacccggtcctactggggaaccccagccacccccttcttctggagctgggggttacagttcagggaggagccagcaggccctaccaccgtggttttcaaactgggttcccactcagcctaggcagtgccagccccacctggggctccggggtggtgcctcaaatccagtggttcagattttctgtttctgcattgacattctgcaaaagattgtgtgtagaataaagggttccttggccaaaaggaattaaagtattgaccccaagcgttttgtttccctccagtttggccaagccaggcttcccagtgcgggaatgaaagtggacgcccgcggctatggtagtcagcaacgctgagggctgactgtgtgccttcactgttcaaggtccccagagggagtctgagcatgagaccagactctctcccaggagctggctgtgggaggcggggaagagggagtgatgaacagtacgaggcacttagcacgcatgggctgtgtctaggcggtggcccccttgaggactcatgacccatggaaaatggctgtgcatctactgtgtgccagcacagtgctgtgcctacatgttcttacctaattgtcattgcaacggagatatgtgttgggctcattttgctgatgaggctcagaggggtgatatgacttgcctaaggccacacagctattgaatagcagagttaggattcaaacccacataggcctacagccaaaatcccaccttcccacctcaccaggccacctgccaatctaatggaggaagcagagtcatttacatacattatttacatatggaaagtgccataacagaggcagtcatttatctgtctagtcaacagatgtttattgagtgcctacgacgtgcaaggcgcagtgctgggagttatgggaacacagagcaggagtgactgccagagctcagggattcatgggcagtttcacagaggggctgccatctgagctgggccctgagtaatagaaagagtttaccagaaggtgacaggtaggggaggagaacgtagtgttaagagcatgggcagcatcagaaagaacgaaatcagccaggaggggtggctcatacctgtaatcccagcactttgggaggcttaggtgggaggattgcttgagtccaggagttcaagaccagcctgagcaacatggagaaactctgtctctacaaaaaacacaaaaattagccaggcgtggtggcatctgctgaggcaggatgattgcttgagcccaggaggtggacactgcagtgagctgagatggcaccactgaactctagcctgggtgaagagtaagaccctgtctcaaaaaaaaaaaaaaaaaaaaaaattaaataatgtcctttgcagcaacctgatggagctggaggcattatcctaagtgaactaaaacagaaaatcaaatactgcatgttctcacgtataggtagaagctaaacagtggatacacatagacacagagatggaaataataggcactgaggacccccaaaatggagagaatgagagcgaggtgagggctggaaaattacctatcgggtacgatgttcattgtttgggtaatggatacactagaagcccagtgcccaccagtaatcaatatacccatgtaataaatatgcatatgtacccccaaatctaaatttttaaatttaaaataaataagagtatggaacttggagtctaatagctgtagtccaagtatagctgggtggccttaggcaaacaattttccacgtctgggtctcagttttctcatttgtaaagtggggctattatttttattttgttttaatttttgagacagagtctcgctctgtcacccaggctggagtgtattggaatgatctcggctcactgaaacctccacttcctgggttcaagcaattctcctgcctcagcctcccaagtagctgggattacaggcacacctggctcattcttgtatttttagtagagacggggtttccccatgttggccaagctggtctcgagctcctgacctcaggtgatccactcgcctaggcctcccaaagtgctgggattacaggcatgagccaccacacccagcaatttttatttttgaggtagagtctcgctctgtcgcccaagctgccaggctggagtgcagtggcacaatcacagctcactgcagccttgacctcctgggctcaagtgatcctcctccctcagcctccaaagtagctgggaccacaggcacgcaccaccatgccaagctaatttttaaaaatactttttttagagatggggtctcaccatgttgcccaggctggtctcaaactcttgggctcaagcgatcctcctgcctcagcctcccaaagtgctgggactacaggtctgagccaccacccctggaggggctattatttccaaaggtcagagaagggtggggccgctcagattgcgctgcaggctgcccgctgctgcctgtgacctgaaccctcacttccctgcaggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 2 - RNA, 2267 nt, Homo sapiens
  source 1..2267: mol_type = mRNA
agccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 3 - protein, 663 aa, Homo sapiens
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP